SEQUENCE LISTING (31 sequences)

SEQ ID NO: 1 - DNA, 1604 bp, Homo sapiens
tgcttggtattcttgggcacgggggctgcaggcacagcacaaagggtaggagagagagctgagtgggggcctggtaaccatcaaaggagaaaatgggctccacaaaggtgggggtggtccattgcttcccccacctcgaaccctaatgactgaatacaaggctagcatggtggggccaaggaaccatcagaccaagcaactaggcatccatccaaaatgagtcattagtggggtggtggggagttgaagtgagggacccctttgcctttgaggcagaatggggaagctagaacacatgagttcctaccagaccctccagctctttctcagcttctcactgccacagcaagcctcgccctggttgttttggagttttttttccctgacatggggaggggttggaagtgggtggggcgattctgaggggggacttggagagagcgggtgagaaagtcgggagcaggttgctatggtaaccgcctcctcagtcaggggaactgttgccagggttactgttagagggggaaaacagaagaaaaagatggtggggggaaggaggtatgacttgtcactctaggctccagcctggctcctagcttcccgtctaccctgactgcctcccctaggtcttccttttctccctccagccctagccacctccagcctccatgttccaggcacatgcctggtagggcagagtggggagtaggagggtagtgccagtgagtaaaccagactccataccttaagctcaactcctatccctttgtcgcctcccaaccccagtcatggctgagtacgggaccctcctgcaagacctgaccaacaacatcacccttgaagatctagaacagctcaagtcggcctgcaaggaagacatccccagcgaaaagagtgaggagatcactactggcagtgcctggtttagcttcctggagagccacaacaagctggacaaaggtgggggaggggagcacaggggtcctgtcatcagtcattcaggctcagttcattcagcaaatagagatgagctcaaagcttttacatccacaatgtgtacccctctatagcaaggcagaagagaggtgctctaagagtatgggggccttagacatttttcactgtgcccctcatcggtaaaaaatgttattcagatactcctgtgctattatcacatatacaacagaacataaaaacaatctaaaaatagtaaaattttaagtggcaaaaattgggaagaattctagacaataaaaatggaagaatacctgaatgaggggagaactcgaagtgtaatctaactggtaaatagtaatactaatctttgttaagcattttttaaaaactggtaaaatttaaaactacaagttctatcattttcttatccactttgagagactactgcttggggagtgggatctatggattagagtaatccctcacagctggaggctttagggaagaattggtgtttaagatggccctgaaaggtgagtgggatttctaaggcaagatccaaaggagggcatgccaggcagaggaacactgtcacagagtatctagtgtgtttggaaatgcaatcagtcctgcttggcttgactagg

SEQ ID NO: 2 - DNA, 3343 bp, Homo sapiens
gacatcgtccttctggtggtgtccataccttctctcatttagaataacctttctcaccaggcccagaatctctcctgggttgcctagagattccaccctgctggtcctacatgtgctgtcagcaaattctgatacttacttggatggaaagtggaaaaggatgggggtgggggtggggtatctggaagtcaagttgatttttcttttgtcccctccccacaacatatgggagctagtatctaagctaaacccctcattctaaccacaatctagaaacttcactgaccaactatctgcccttcctgcgaacagtcctctgtattgttgtttgggtaccttagctttcttctcctggattagtagcccctcttcctccagtgttgtaccctcccataaccaatgtcagcaactcagctttggttccaggtcactagtctggtggagtaggggaagctgacctctacagcctagctctgaccctatctcctgccttcctccagacaacctctcctacattgagcacatctttgagatctcccgccgtcctgacctactcactatggtggttgactacagaacccgtgtgctgaagatctctgaggaggatgagctggacaccaagctaacccgtatccccagtgccaagaagtacaaaggtaagcggccactcctttaactagctgcacctctgcctcgtcccgttgactatccttggagtacttgagttttgggagagtggaggcagatgcccaatgggcctgcctggcatctcccacactgctgtccctggacacatacctttttgcccccagacattatccggcagccctctgaggaagagatcatcaaattggctcccccaccgaagaaggcctgagcaagggggaggaagaggaggaaggttggaccttcatcagaccactcccttcccccatcctccaggagagggggcaagggcaacccaccatctacccacttactaacctggtcctaacccccttactgtgcgcgtgtgtgtgcgtgtgcgcacgctctggctgtttgtctatatgtctagctcatctagttcctcttcttaaggggatgggggtcaggggctaggggagggggctgagtttccccactttaggaggaggtgggggctatttctatgcaaatagaaatcagcacattcctcctacttccctttcctccactccccccatatctttaaagtgtggaagcagaaaggacctgcattttcctacattgaggagctgacataggggtaaggtatgggagaggtaggtggatccagggaaaagcagtggggacggaaggcaaagagaccactcaacccccacctggaaggggcaaagaaaagccagagttccatgtttgtactcctgtgctggactgtttcctgagtaccagcaggtccctttttgtctctcatgggcctagcataggtatgagccagggatcctttcctggtccctaagatcaaaccccatggagcagccagcgttagatgcccccacccacctgtactctggagagactgtgctgggaacatgtaccactgagcctgagatggggatgagggcagagagaggggagccccctcttccactcagttgttcctactcagactgttgcactctaaacctagggaggttgaagaatgagacccttaggttttaacacgaatcctgacaccaccatctatagggtcccaacttggttattgtaggcaaccttccctctctccttggtgaagaacatcccaagccagaaagaagttaactacagtgttttcctttgcaccgatccccaccccaattcaatcccggaagggacttacttaggaaacccttctttactagatatcctggccccctgggcttgtgaacacctcctagccacatcactacagtacagtgagtgaccccagcctcctgcctaccccaagatgcccctccccaccctgaccgtgctaactgtgtgtacatatatattctacatatatgtatattaaaactgcactgccatgtctgcccttttttgtggtgtctagcattaacttattgtctaggccagagcgggggtgggaggggaatgccacagtgaagggagtggcagaatcaaattgctacatagtccaaacaaaaaagaaggctttttcaaaaaacattaaattcacatgcagtctcagagactatttagacaaagttcaagttaggagcttttaggatgtgggagtaaaactttaatgggaggggagggctggctgctggaagaaggaagaagccagactggttagacagtactcttaactcctagcccagcctagcgtgccctgcccctctggccactgctgcagacacctgccttaacacacacacctctaggactccacagttttgccttaaaggaccttcccaagtctccctttccctgtctggcttctcccttaagaagagagagatacttgtagaattgggtggggggaatgagcatgaactgtccttccatttgggatatgttacattagagtgagagagagaataaggagcctttcttatggaagaaatgggagaagagagacagggttcttttcagcagagtctagtagtttctctgtaaggcaaaataatctaaaaagactaacctgcccacccactccttatattgctgtgagattgcccctatcttgtgctcttctgtctgcagtgtgcacggccttgttctaacccggaataaaggtgattgattgtattgcaactaactgattttaaaatttctgtgagtgaccaggattttgcaggctttgaatcactgggaagggagggggaacaatatttatggcccagaataagggctgggaaaataccccaagtgtataccctcaccccacccaatctcctaagtatcactgagctaaatggagtgtatcctacttccaacagtctccaattggagcccaagcactggaaatacgggtctcccaaaatggactttctaacactgtatcatgtgcagaatgagaggggaaggctaggtacctacattagtaggaacaaaaaccatccattgtgaataaatggaccacataccacacagagcagcagtcgggagaagaaaagctttactgggagaaaatacaacaaattccagagtgcatggtttttagcccaccctatcaccccaccagcaataggaacacagaccactcgatcaccacacattccctacctcagggagtaagtacagcagccaacatctggtctcagagctgctgggaaaaggggcaggaggaaagaagtatctgggaataccattctctcactcttttcccctcctt

SEQ ID NO: 3 - DNA, 3072 bp, Homo sapiens
  misc_feature (2978)..(2978): n is a, c, g, or t
taatatatacaaatagctaaacaagcccataaataataaacattatatatatatttttgagatgatgcccataaataataaacattatatatatatacatatacatatatatgtgtgtgtatatatatatatttttttctttttttctttttttttgagatggagtctcactctgttgccaggttggagtgcagtggtgagatctcggttcactgcaacctccgactcccgggttcaagagattctcctatctcagcctcccaggtaactgggactacaggtgcccaccaccacactcagctaacttttttgtagttttagtagagatggggtttcaccatgttggtcaggctagtcttgaactcctgacctcaggtgatccacctgcctcagcctcccaaaatgctgggattacaggcgtgagccaccacgccaagccaacattatatttttcatggatcaggtgtaaagttgttgctgagagacttagaggatttctaagtacggaaagatgagacctgagattagatatattttcactctccacagacaagtccaggaagtgttcctagagaatgtggcaggggtgtagaaagcagaaactatgtattcattccaacttgtagctatttaataaatgtttaataagtagtatgggcactaagctagtcttacaataatattgtggagcttactgtctagcataggtcagtggaaggccccggtctaaataatagctcctattttttaagcacccatctgtgccagtccctagagacttactatttctgaatctcagaagaaccctcagaaatagacattggcaccctgatctgaccggtgaggacactaaggctttcaggcataacttgcccaaggaatgggtggaactagcacgcagcccaaggcccctgtgtcttgttgcctccagacctgagcctttcacttagaaactacatgagtctcaaagatgggtcaacgaaatagtttttgtccctacaacacagagcaaacatgatatatacaaaggcaaatgattagtgtgtgacagggaatgctggggagaggaaacagcagagaaatgctatgaaacccagagcagcaggaacttgggcctcttctaggaatggtgtagacccaagaaaggaggggctcgtttgaggtttctggaggagtctctgccaggtaggaactgagatgagatgggtgatcctggaagtggcacaaacactgaggggaatcccccaacctgggcctgctggagttcctggcaggaaggacctacccagggtcagacttctttccagggccaaaccctgcccagagctgaggcaagaccccttaggctatagagatgggggtgaaaacagatgcttctccaattcttcctttcctcttttcagtctggctgagaatctgggggaggaaagtccctggggtagaggggaagtgagagctgccacattgggcctgggcagaggggcagagctaggagatggagctaagtgtggctttgcaagttgtctatatttgcatgctgagctcactgctgctctttttctccccagaaaataaaattacatcatggtagggggaaggcaggagtggggcatgtttgggaaaggtgagaggcacgcaagagggcctgaaggcatcatcctggaaagagaagatccagggctcctcaagtccccatgtggccccaacccctcactcccaatgtggataagtctgatcagtcattttccactgcgcccctgacccttaaggcaaggcaagaagtttttacaaatattaaagccagcagctaagagaatccagcccccaaaggctttccaaccccctaaaccaaacctactctgtgcctcccaggggtcctaggcaccgtggtcatccaaaggtcaaaaggggaggagggtgccctactgggtaagaattaaatatgagaattgtttcgcagttcagggggaagagaccgtctccgctccagccctccgcccaattctgtcccccatcctaggagtcttccagaaccccagcccctcggtcccccgccccctcctgcctcttccatccctccccctcctcccgccattccccgccccccgcccctccccggctctgacatcaccggccagccgggtggagtgagcggcgctgggctcgggctccggctccgcgggcggaagaggcggcggcggcggcagaagcggcggcggcggcggcgggagccgaggaggaggttccggacgctgcttaggaaccggggactcaggagtgcccgcgccctgagcgctcagctccagaggcggtgagaggggcggagaggagacatgtcgcggggaaggggcagatttcggggtctaggcttggaggggcaacgatctgggacaccgggggccagacggggagtgtggggaccccggacgagtccactttgtgtcaggcctcacggggtccgtccctcggtcggtgcgtcccgagcgccgagcgtgtgctggaggcggcgtggctccggccagcccgttccacgctgaacggcagtttgggagtcggaccggggcctgtggattaggatgcagcctgcttcccgcccagccggctcggccaggccagcgcccgcccctccctccccgctcaggcttcggactcgcctccgctggcggtctcccggcccagccctgtctcggttccgcgggcggctggtaccatgtgccctcacccccagctcactgcacctggggaagaggtgggcttggcggaaagcttgtccagagggaaggaggagctgggggtgtagagaagtgatgtatatctaacgtaaaaacccttattaatttcatataatccgtgtcaaaactgacagggaaacacgcacacctataaggcacgccagcacacacagtgacacacacccatgtgacttatacacatagtctagcacactccaaatcacaaacgtccctggactacgcatncggccgcccaccacacccccactctgtggggtggggcatcccatgaggagtcttctgaactgcgggctccaagtcatcagtccgacaggacagc

SEQ ID NO: 4 - DNA, 190 bp, Mus musculus
tccaagcaagtagttaattacagtgtttcctttgcactgaccaccaccctgattcaatccaggaagggactggtaacctttctcatttgggtttgtggatgccacacagccaagtcactagagtgcagtagtaacccagcctctcctgtccaagatgccttccatcttgacgtgctaattgtgtgaagtc

SEQ ID NO: 5 - DNA, 1637 bp, Homo sapiens
gccgaggaggaggttccggacgctgcttaggaaccggggactcaggagtgcccgcgccctgagcgctcagctccagaggcgtcatggctgagtacgggaccctcctgcaagacctgaccaacaacatcacccttgaagatctagaacagctcaagtcggcctgcaaggaagacatccccagcgaaaagagtgaggagatcactactggcagtgcctggtttagcttcctggagagccacaacaagctggacaaagacaacctctcctacattgagcacatctttgagatctcccgccgtcctgacctactcactatggtggttgactacagaacccgtgtgctgaagatctctgaggaggatgagctggacaccaagctaacccgtatccccagtgccaagaagtacaaagacattatccggcagccctctgaggaagagatcatcaaattggctcccccaccgaagaaggcctgagcaagggggaggaagaggaggaaggttggaccttcatcagaccactcccttcccccatcctccaggagagggggcaagggcaacccaccatctacccacttactaacctggtcctaacccccttactgtgcgcgtgtgtgtgcgtgtgcgcacgctctggctgtttgtctatatgtctagctcatctagttcctcttcttaaggggatgggggtcaggggctaggggagggggctgagtttccccactttaggaggaggtgggggctatttctatgcaaatagaaatcagcacattcctcctacttccctttcctccactccccccatatctttaaagtgtggaagcagaaaggacctgcattttcctacattgaggagctgacataggggtaaggtatgggagaggtaggtggatccagggaaaagcagtggggacggaaggcaaagagaccactcaacccccacctggaaggggcaaagaaaagccagagttccatgtttgtactcctgtgctggactgtttcctgagtaccagcaggtccctttttgtctctcatgggcctagcataggtatgagccagggatcctttcctggtccctaagatcaaaccccatggagcagccagcgttagatgcccccacccacctgtactctggagagactgtgctgggaacatgtaccactgagcctgagatggggatgagggcagagagaggggagccccctcttccactcagttgttcctactcagactgttgcactctaaacctagggaggttgaagaatgagacccttaggttttaacacgaatcctgacaccaccatctatagggtcccaacttggttattgtaggcaaccttccctctctccttggtgaagaacatcccaagccagaaagaagttaactacagtgttttcctttgcaccgatccccaccccaattcaatcccggaagggacttacttaggaaacccttctttactagatatcctggccccctgggcttgtgaacacctcctagccacatcactacagtacagtgagtgaccccagcctcctgcctaccccaagatgcccctccccaccctgaccgtgctaactgtgtgtacatatatattctacatatatgtatattaaaactgcactgccatgtctgcc

SEQ ID NO: 6 - DNA, 2391 bp, Mus musculus
caggatcggcggcagcggtggcaggagccaaggagcaggctccagacgctgcttaggaaccggggacccgggagtgcccgcaccctgagctctcagctccggaggcgtcatggcagagtacggaactctccttcaggacctgaccaacaacatcacccttgaagatctggaacagctcaagtcagcctgcaaggaggacatccccagtgagaagagtgaggagatcaccacaggcagcgcctggtttagcttcctggagagccacaacaagctggacaaagacaacctctcctacatagagcacatctttgagatatctcgccgtcctgacctcctcactatggtggttgactacagaacccgtgtgctgaagatctctgaggaggaggagctggacaccaagctaacccgtattcccagtgccaagaagtacaaagacattatccggcagccctctgaagaagaaatcatcaaattggctcccccaccaaagaaggcctgaacaagggggaggaagaggaggaaggttggatcttcatcagaccactcccttccccatcctcaatgggaggggctagggcaaccccctgctccgtacccatttactaacttggtcctaacccttactatgcgcgtgtgtgtcggtgtgcgcacgctctggctgtctgtctgtctagctcatctagttcctcctcctcatgaggggattggaggcaagggagggggagcttaattccccccactatagggggaggtggacgctttttctatgtaaacagaaattggcacattcctcctccttctcccctttcctccactcttcccccacatctttatggaagcagaaaggacctgcattttcctacactgaggagctatggtgaagatgaggtatgggagagatggttgtatctaaagaaaaccagtggggaaggaaggtaaagaggccactcaacctccacctggtaagggacaaagaaaagccaggactcagtgtttgtatctctatgctggactggttagaagccagcttcccgctgttcccttaggctgaggtcctgagtgccaatgggccctccttatgcccttgttatgggcctgggatgcggatgagccagaaatcttaatgagaagaccactcgatcctttcctggtcccaaagatcaaaccccaatggagaagccagcatttactgtcccccaaccttctgctctggagagacgatgccgggaccatgcactactgagcctgagctagggacgcaggcagagacaggcccacttcctgctcctcagtttctaaatacagactgttggataaactgacctggagcctagggagattgggggatgagatccttaggttttaaccccaacctgcccacaacctacagagtattgtaggcaacctttccactctccagtttagaattctccaagcaagtagttaattacagtgtttcctttgcactgaccaccaccctgattcaatccaggaagggactggtaacctttctcatttgggtttgtggatgccacacagccaagtcactagagtgcagtgagtgaacccagcctcctccctgtcccaagatgcccttccccatcttgaccgtgctaactgtgtgtacatatatattctacatatatgtatattaaacccgcactgccatgtctgcccttttttgtggtttttagcattaacttattgtctaggccagagcgggagtgggaggggatgccacagaaaggaagtggcagagccaaattgttacagagtccaaacagaaaacacatttcaactaaccacaacaaatgttacatttacatgtgagctaagagacaatttagagaaagatgaaggtaggtgcttttaggatgtgggagcaaaacttcacagggaggggaggaatggctgtcagaagagggttcttgaagccagactgactagagcaccctcagttcctagctgagcccagattgcccatctcctctggccactgctgcagacacctgccttaactctcacacctcgaagactccagttttgccttaaaggtccttcctaaatctccctagtcttgcctggcatctcctttaagacaagaaatccttgtacaactgggtgggagaaaggaactctgggtaacatccttccttctggggtgttccatgggagcaggagtgagacggcgcttcccttatggaagagggaggagacagggtgcttctcagaagcttctctgtaaggcaaaaaccaaactttaaacagactaacctgccctaatatactcaccccctcgtgctgctgtgaggttgctcctagcctgtgctcctctgtctgcagcgtgcacagccttgttctaaccctggaataaaggtgactgactct

SEQ ID NO: 7 - DNA, 522 bp, Canis familiaris
  misc_feature (210)..(210): n is a, c, g, or t
ttacagtacagtcaatcacctttattccagggttagaacaaggccgtgcacactgcagacaggagcacaggatggggacaatctcacagccgtacagggtgggagggcaggttagtctttttaggttatttttgccttacagagagatttctagactctgctggaaatgaccctgtctcccttctcccatctcccccctcctctcctcanagaaggctcctattccttcctcctcttccccctctcgctctcgctcgctctcacgcacattctcacatcacgtaatcctgaccggaaggacagtcgtgctcagctcccccctcccaattctacaagtgcctctcggtcctggagggagaagcagacagggctggggcatctcggggaggtcctttaaggcaaaactgnggagtactccaggtgtccgtgttaaggcaggtgtccccancantggccaaaagggacggctgggctgggctggctgggcgtgcaaggttgttccnggcccgnnggtttcttctt

SEQ ID NO: 8 - DNA, 1676 bp, Mus musculus
ccacgcgtccgagccaaggagcaggctccagacgctgcttaggaaccggggacccgggagtgcccgcaccctgagctctcagctccggaggcgtcatggcagagtacggaactctccttcaggacctgaccaacaacatcacccttgaagatctggaacagctcaagtcagcctgcaaggaggacatccccagtgagaagagtgaggagatcaccacaggcagtgcctggtttagcttcctggagagccacaacaagctggacaaagacaacctctcctacattgagcacatctttgagatatctcgccgtcctgacctcctcactatggtggttgactacagaacccgtgtgctgaagatctctgaggaggaggagctggacaccaagctaacccgtattcccagtgccaagaagtacaaagacattatccggcagccctctgaagaagaaatcatcaaattggctcccccaccaaagaaggcctgaacaagggggaggaagaggaggaaggttggatcttcatcagaccactcccttccccatcctcaatgggaggggctagggcaaccccctgctccgtacccatttactaacttggtcctaacccttactatgcgcgtgtgtgtgcgtgtgcgcacgctctggctgtctgtctgtctagctcatctagttcctcctcctcatgaggggattggaggcaagggagggggggcttaaatccccccactatagggggaggtggacgctttttctatgtaaacagaaattggcacattcctcctccttctcccctttcctccactcttcccccacatctttatggaagcagaaaggacctgcattttcctacactgaggagctatggtgaagatgaggtatgggagagatggttgtatctaaagaaaaccagtggggaaggaaggtaaagaggccactcaacctccacctggtaagggacaaagaaaagccaggactcagtgtttgtatctctatgctggactggttagaagccagcttcctgctgttcccttaggctgaggtcctgagtgccaatgggccctccttatgcccttgttatgggcctgggatgcggatgagccagaaatcttaatgagaagaccactcgatcctttcctggtcccaaagatcaaaccccaatggagaagccagcatttactgtcccccaaccttctgctctggagagacgatgccgggaccatgcactactgagcctgagctagggacgcaggcagagacaggcccactttctgctcctcagtttctaaatacagactgttggataaactgacctggagcctagggagattgggggatgagatccttaggttttaaccccaacctgcccacaacctacagagtattgtaggcaacctttccactctccagtttagaattctccaagcaagtagttaattacagtgtttcctttgcactgaccaccaccctgattcaatccaggaagggactggtaacctttctcatttgggtttgtggatgccacacagtcaagtcactagagtgcagtgagtgaacccagcctcctccctgtcccaagatgcccttccccatcttgaccgtgctaactgtgtgtacatatatattctacatatatgtatattaaacccgcactgccatgtcaaaaaaaaaaaaaaa

SEQ ID NO: 9 - DNA, 393 bp, Homo sapiens
atggctgagtacgggaccctcctgcaagacctgaccaacaacatcacccttgaagatctagaacagctcaagtcggcctgcaaggaagacatccccagcgaaaagagtgaggagatcactactggcagtgcctggtttagcttcctggagagccacaacaagctggacaaagacaacctctcctacattgagcacatctttgagatctcccgccgtcctgacctactcactatggtggttgactacagaacccgtgtgctgaagatctctgaggaggatgagctggacaccaagctaacccgtatccccagtgccaagaagtacaaagacattatccggcagccctctgaggaagagatcatcaaattggctcccccaccgaagaaggcctga

SEQ ID NO: 10 - DNA, 221 bp, Pan troglodytes
acaacctctcctacattgagcatatctttgagatctcccgccgtcctgacctactcactatggtggttgactacagaacccgtgtgctgaagatctctgaggaggatgagctggacaccaagctaacccgtatccccagtgccaagaagtacaaagacattatccggcagccctctgaggaagagatcatcaaattggctcccccaccgaagaaggcctga

SEQ ID NO: 11 - DNA, 393 bp, Mus musculus
atggcagagtacggaactctccttcaggacctgaccaacaacatcacccttgaagatctggaacagctcaagtcagcctgcaaggaggacatccccagtgagaagagtgaggagatcaccacaggcagtgcctggtttagcttcctggagagccacaacaagctggacaaagacaacctctcctacatagagcacatctttgagatatctcgccgtcctgacctcctcactatggtggttgactacagaacccgtgtgctgaagatctctgaggaggaggagctggacaccaagctaacccgtattcccagtgccaagaagtacaaagacattatccggcagccctctgaagaagaaatcatcaaattggctcccccaccaaagaaggcctga

SEQ ID NO: 12 - DNA, 1770 bp, Mus musculus
gtcgacggccattaccaatcgcgaccggcaagggagggggagcttaattccccccactatagggggaggtggacgctttttctatgtaaacagaaattggcacattcctcctccttctcccctttcctccactcttcccccacatctttatggaagcagaaaggacctgcattttcctacactgaggagctatggtgaagatgaggtatgggagagatggttgtatctaaagaaaaccagtggggaaggaaggtaaagaggccactcaacctccacctggtaagggacaaagaaaagccaggactcagtgtttgtatctctatgctggactggttagaagccagcttcccgctgttcccttaggctgaggtcctgagtgccaatgggccctccttatgcccttgttatgggcctgggatgcggatgagccagaaatcttaatgagaagaccactcgatcctttcctggtcccaaagatcaaaccccaatggagaagccagcatttactgtcccccaaccttctgctctggagagacgatgccgggaccatgcactactgagcctgagctagggacgcaggcagagacaggcccactttctgctcctcagtttctaaatacagactgttggataaactgacctggagcctagggagattgggggatgagatccttaggttttaaccccaacctgcccacaacctacagagtattgtaggcaacctttccactctccagtttagaattctccaagcaagtagttaattacagtgtttcctttgcactgaccaccaccctgattcaatccaggaagggactggtaacctttctcatttgggtttgtggatgccacacagccaagtcactagagtgcagtgagtgaacccagcctcctccctgtcccaagatgcccttccccatcttgaccgtgctaactgtgtgtacatatatattctacatatatgtatattaaacccgcactgccatgtctgcccttttttgtggtttttagcattaacttattgtctaggccagagcgggagtgggaggggatgccacagaaaggaagtggcagagccaaattgttacagagtccaaacagaaaacacatttcaactaaccacaacaaatgttacatttacatgtgagctaagagacaatttagagaaagatgaaggtaggtgcttttaggatgtgggagcaaaacttcacagggaggggaggaatggctgtcagaagagggttcttgaagccagactgactagagcaccctcagttcctagctgagcccagattgcccatctcctctggccactgctgcagacacctgccttaactctcacacctcgaagactccagttttgccttaaaggtccttcctaaatctccctagtcttgcctggcatctcctttaagacaagaaatccttgtacaactgggtgggagaaaggaactctgggtaacatccttccttctggggtgttccatgggagcaggagtgagacggcgcttcccttatggaagagggaggagacagggtgcttctcagaagcttctctgtaaggcaaaaaccaaactttaaacagactaacctgccctaatatactcaccccctcgtgctgctgtgaggttgctcctagcctgtgctcctctgtctgcagcgtgcacagccttgttctaaccctggaataaaggtgactgactctaccgtaaaaaaaaaaaaaaaaaaaaaaaaaaaaaaaaaaaaaaaaggccgcctaggcccgtcgac

SEQ ID NO: 13 - DNA, 393 bp, Mus musculus
atggcagagtacggaactctccttcaggacctgaccaacaacatcacccttgaagatctggaacagctcaagtcagcctgcaaggaggacatccccagtgagaagagtgaggagatcaccacaggcagcgcctggtttagcttcctggagagccacaacaagctggacaaagacaacctctcctacatagagcacatctttgagatatctcgccgtcctgacctcctcactatggtggttgactacagaacccgtgtgctgaagatctctgaggaggaggagctggacaccaagctaacccgtattcccagtgccaagaagtacaaagacattatccggcagccctctgaagaagaaatcatcaaattggctcccccaccaaagaaggcctga

SEQ ID NO: 14 - DNA, 115 bp, Sus scrofa STS
gacaaaagtcatgggaggcaccaggcaggccaactgagcatcagcctctgttcttccaaaattcaagtgctccacagacaaccatgggagggggcagaggtgcgtatgaaaggca

SEQ ID NO: 15 - DNA, 935 bp, Homo Sapiens
gggcttgtgaacacctcctagccacatcactacagtacagtgagtgaccccagcctcctgcctaccccaagatgcccctccccaccctgaccgtgctaactgtgtgtacatatatattctacatatatgtatattaaaactgcactgccatgtctgcccttttttgtggtgtctagcattaacttattgtctaggccagagcgggggtgggaggggaatgccacagtgaagggagtggcagaatcaaattgctacatagtccaaacaaaaaagaaggctttttcaaaaaacattaaattcacatgcagtctcagagactatttagacaaagttcaagttaggagcttttaggatgtgggagtaaaactttaatgggaggggagggctggctgctggaagaaggaagaagccagactggttagacagtactcttaactcctagcccagcctagcgtgccctgcccctctggccactgctgcagacacctgccttaacacacacacctctaggactccacagttttgccttaaaggaccttcccaagtctccctttccctgtctggcttctcccttaagaagagagagatacttgtagaattgggtggggggaatgagcatgaactgtccttccatttgggatatgttacattagagtgagagagagaataaggagcctttcttatggaagaaatgggagaagagagacagggttcttttcagcagagtctagtagtttctctgtaaggcaaaataatctaaaaagactaacctgcccacccactccttatattgctgtgagattgcccctatcttgtgctcttctgtctgcagtgtgcacggccttgttctaacccggaataaaggtgattgattgtattgcaaaaaaaaaaaaaaaaaaaaaaaaaaaaaaaaaaaaaaaaaaaaaaaaaaaaaa

SEQ ID NO: 16 - DNA, 2486 bp, Homo Sapiens
ggcatcaccggccagccgggtggagtgagcggcgctgggctcgggctccggctccgcgggcggaagaggcggcggcggcggcagaagcggcggcggcggcggcggcgggagccgaggaggaggttccggacgctgcttaggaaccggggactcaggagtgcccgcgccctgagcgctcagctccagaggcgtcatggctgagtacgggaccctcctgcaagacctgaccaacaacatcacccttgaagatctagaacagctcaagtcggcctgcaaggaagacatccccagcgaaaagagtgaggagatcactactggcagtgcctggtttagcttcctggagagccacaacaagctggacaaagacaacctctcctacattgagcacatctttgagatctcccgccgtcctgacctactcactatggtggttgactacagaacccgtgtgctgaaaatctctgaggaggatgagctggacaccaagctaacccgtatccccagtgccaagaagtacaaagacattatccggcagccctctgaggaagagatcatcaaattggctcccccaccgaagaaggcctgagcaagggggaggaagaggaggaaggttggaccttcatcagaccactcccttcccccatcctccaggagagggggcaagggcaacccaccatctacccacttactaacctggtcctaacccccttactgtgcgcgtgtgtgtgcgtgtgcgcacgctctggctgtttgtctatatgtctagctcatctagttcctcttcttaaggggatgggggtcaggggctaggggagggggctgagtttccccactttaggaggaggtgggggctatttctatgcaaatagaaatcagcacattcctcctacttccctttcctccactccccccatatctttaaagtgtggaagcagaaaggacctgcattttcctacattgaggagctgacataggggtaaggtatgggagaggtaggtggatccagggaaaagcagtggggacggaaggcaaagagaccactcaacccccacctggaaggggcaaagaaaagccagagttccatgtttgtactcctgtgctggactgtttcctgagtaccagcaggtccctttttgtctctcatgggcctagcataggtatgagccagggatcctttcctggtccctaagatcaaaccccatggagcagccagcgttagatgcccccacccacctgtactctggagagactgtgctgggaacatgtaccactgagcctgagatggggatgagggcagagagaggggagccccctcttccactcagttgttcctactcagactgttgcactctaaacctagggaggttgaagaatgagacccttaggttttaacacgaatcctgacaccaccatctatagggtcccaacttggttattgtaggcaaccttccctctctccttggtgaagaacatcccaagccagaaagaagttaactacagtgttttcctttgcaccgatccccaccccaattcaatcccggaagggacttacttaggaaacccttctttactagatatcctggccccctgggcttgtgaacacctcctagccacatcactacagtacagtgagtgaccccagcctcctgcctaccccaagatgcccctccccaccctgaccgtgctaactgtgtgtacatatatattctacatatatgtatattaaaactgcactgccatgtctgcccttttttgtggtgtctagcattaacttattgtctaggccagagcgggggtgggaggggaatgccacagtgaagggagtggcagaatcaaattgctacatagtccaaacaaaaaagaaggctttttcaaaaaacattaaattcacatgcagtctcagagactatttagacaaagttcaagttaggagcttttaggatgtgggagtaaaactttaatgggaggggagggctggctgctggaagaaggaagaagccagactggttagacagtactcttaactcctagcccagcctagcgtgccctgcccctctggccactgctgcagacacctgccttaacacacacacctctaggactccacagttttgccttaaaggaccttcccaagtctccctttccctgtctggcttctcccttaagaagagagagatacttgtagaattgggtggggggaatgagcatgaactgtccttccatttgggatatgttacgttagagtgagagagagaataaggagcctttcttatggaagaaatgggagaagagagacagggttcttttcagcagagtctagtagtttctctgtaaggcaaaataatctaaaaagactaacctgcccacccactccttatattgctgtgagattgcccctatcttgtgctcttctgtctgcagtgtgcacggccttgttctaacccggaataaaggtgattgattgtattgcaaaaaaaaaaaaaaaa

SEQ ID NO: 17 - DNA, 2486 bp, Homo Sapiens; identical to SEQ ID NO: 16

SEQ ID NO: 18 - DNA, 1674 bp, Homo Sapiens
ccgaggaggaggttccggacgctgcttaggaaccggggactcaggagtgcccgcgccctgagcgctcagctccagaggcgtcatggctgagtacgggaccctcctgcaagacctgaccaacaacatcacccttgaagatctagaacagctcaagtcggcctgcaaggaagacatccccagcgaaaagagtgaggagatcactactggcagtgcctggtttagcttcctggagagccacaacaagctggacaaagacaacctctcctacattgagcacatctttgagatctcccgccgtcctgacctactcactatggtggttgactacagaacccgtgtgctgaagatctctgaggaggatgagctggacaccaagctaacccgtatccccagtgccaagaagtacaaagacattatccggcagccctctgaggaagagatcatcaaattggctcccccaccgaagaaggcctgagcaagggggaggaagaggaggaaggttggaccttcatcagaccactcccttcccccatcctccaggagagggggcaagggcaacccaccatctacccacttactaacctggtcctaacccccttactgtgcacgtgtgtgtgcgtgtgcgcacgctctggctgtttgtctatatgtctagctcatctagttcctcttcttaaggggatgggggtcaggggctaggggagggggctgagtttccccactttaggaggaggtgggggctatttctatgcaaatagaaatcagcacattcctcctacttccctttcctccactccccccatatctttaaagtgtggaagcagaaaggacctgcattttcctacattgaggagctgacataggggtaaggtatgggagaggtaggtggatccagggaaaagcagtggggacggaaggcaaagagaccactcaacccccacctggaaggggcaaagaaaagccagagttccatgtttgtactcctgtgctggactgtttcctgagtaccagcaggtccctttttgtctctcatgggcctagcataggtatgagccagggatcctttcctggtccctaagatcaaaccccatggagcagccagcgttagatgcccccacccacctgtactctggagagactgtgctgggaacatgtaccactgagcctgagatggggatgagggcagagagaggggagccccctcttccactcagttgttcctactcagactgttgcactctaaacctagggaggttgaagaatgagacccttaggttttaacacgaatcctgacaccaccatctatagggtcccaacttggttattgtaggcaaccttccctctctccttggtgaagaacatcccaagccagaaagaagttaactacagtgttttcctttgcaccgatccccaccccaattcaatcccggaagggacttacttaggaaacccttctttactagatatcctggccccctggtcttgtgaacacctcctagccacatcactacagtacagtgagtgaccccagcctcctgcctaccccaagatgcccctccccaccctgaccgtgctaactgtgtgtacatatatattctacatatatgtatattaaaactgcactgccatgtctgccaaaaaaaaaaaaaaaaaaaaaaaaaaaaaaaaaaaaaa

SEQ ID NO: 19 - protein, 130 aa, Homo Sapiens
MAEYGTLLQDLTNNITLEDLEQLKSACKEDIPSEKSEEITTGSAWFSFLESHNKLDKDNLSYIEHIFEISRRPDLLTMVVDYRTRVLKISEEDELDTKLTRIPSAKKYKDIIRQPSEEEIIKLAPPPKKA

SEQ ID NO: 20 - protein, 130 aa, Homo sapiens; identical to SEQ ID NO: 19

SEQ ID NO: 21 - protein, 130 aa, Homo sapiens; identical to SEQ ID NO: 19

SEQ ID NO: 22 - protein, 130 aa, Homo sapiens; identical to SEQ ID NO: 19

SEQ ID NO: 23 - protein, 130 aa, Mus musculus
MAEYGTLLQDLTNNITLEDLEQLKSACKEDIPSEKSEEITTGSAWFSFLESHNKLDKDNLSYIEHIFEISRRPDLLTMVVDYRTRVLKISEEEELDTKLTRIPSAKKYKDIIRQPSEEEIIKLAPPPKKA

SEQ ID NO: 24 - protein, 130 aa, Mus musculus; identical to SEQ ID NO: 23

SEQ ID NO: 25 - protein, 61 aa, Mus musculus
MYIKPALPCLPFFVVFSINLLSRPEREWEGMPQKGSGRAKLLQSPNRKHISTNHNKCYIYM

SEQ ID NO: 26 - protein, 130 aa, Mus musculus; identical to SEQ ID NO: 23

SEQ ID NO: 27 - protein, 130 aa, Homo sapiens; identical to SEQ ID NO: 19

SEQ ID NO: 28 - protein, 74 aa, Homo sapiens
MYIKTALPCLPFFVVSSINLLSRPERGWEGNATVKGVAESNCYIVQTKKKAFSKNIKFTCSLRDYLDKVQVRSF

SEQ ID NO: 29 - protein, 130 aa, Homo sapiens; identical to SEQ ID NO: 19

SEQ ID NO: 30 - protein, 130 aa, Homo sapiens; identical to SEQ ID NO: 19

SEQ ID NO: 31 - DNA, 21 bp, Artificial Sequence;
  Synthetic Primer
ggaagacatccccagcgaatt

(19) United States
(12) Patent Application Publication
(10) Pub. No.: US 2009/0233848 A1
(43) Pub. Date: Sep. 17, 2009

(54) Pea15 as a Tumor Suppressor Gene

(76) Inventors: Chandra Bartholomeusz, Houston, TX (US); Naoto T. Ueno, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/592,276

(22) PCT Filed: Mar. 3, 2005
(86) PCT No.: PCT/US05/06978

§ 371 Date: May 9, 2007

Related U.S. Application Data
(60) Provisional application No. 60/551,608, filed on Mar. 9, 2004.

(51) Int. Cl. A61K 38/16; C12N 15/64; A61P 35/00

(52) U.S. Cl. 514/12; 435/320.1

(57) ABSTRACT

The present invention relates generally to the fields of cancer therapy and gene therapy. More particularly, the invention is directed to PEA15 as a treatment for cancer. In a particular embodiment, PEA15 acts as a tumor suppressor for treatment of the cancer. In another particular embodiment, the cancer cells comprises E1 tumor suppressor activity. In another embodiment, the tumorigenicity of the cells is associated with ERK-dependent transcription and proliferation. In a further embodiment, PEA15 facilitates sensitivity of a tumor cell to a chemotherapeutic, such as paclitaxel.

### REFERENCE TO RELATED APPLICATION

The present application is a national phase application under 35 U.S.C. § 371 of International Application No. PCT/US05/06978, filed Mar. 3, 2005, and U.S. Provisional Patent Application Ser. No. 60/551,608, filed on Mar. 9, 2004, which are both incorporated by reference herein in their entirety.

### STATEMENT REGARDING FEDERALLY SPONSORED RESEARCH OR DEVELOPMENT

Federal funds pursuant to NIH Grant CA76450-1 and CA16672-27 were utilized in the invention. The United States Government may have certain rights in the invention.

### FIELD OF THE INVENTION

The present invention relates generally to the fields of cell biology, molecular biology, cancer therapy, and gene therapy. More particularly, it concerns the use of phosphoprotein-enriched in astrocytes (PEA15) to treat cancer, wherein in particular embodiments it comprises tumor suppressor activity.

### BACKGROUND OF THE INVENTION

Cancer remains a leading health issue for millions of individuals. For example, advanced breast and ovarian cancer remain among the leading causes of cancer death in women in the United States. New therapeutic modalities based on molecular anti-cancer mechanisms are urgently needed. The administration of tumor suppressor genes is one useful strategy for the prevention and/or treatment of cancer.

I. Adenovirus Type 5 E1A Functions as a Tumor Suppressor Gene

The adenovirus type 5 E1A gene is the first viral gene expressed in cells after the virus infection and is a well-known transcription factor (Nevins et al., 1988; Nevis, 1990). The HER-2/neu gene, which encodes a Mr 185-kDa transmembrane growth factor receptor with tyrosine kinase (Dunton, 1997; Coussens et al., 1985; Schechter et al., 1985; Bargmann et al., 1986), is overexpressed in 15-20% of human ovarian and breast cancers and its expression is associated with poor outcome. Adenovirus type 5 E1A was utilized as a therapeutic agent for the HER2-overexpressing ovarian and breast cancer cells based on previous findings that showed E1A to be not only a transcription repressor of HER2 (Yu et al., 1990; Yan et al., 1991) but also a potent inhibitor of HER2-mediated transformation, tumorigenicity, and metastasis in rodent cells (Yu et al., 1992; Yu and Hung, 1991) and in human ovarian cancer cells that overexpress HER2 (Yu et al., 1994). To test the efficacy of E1A gene therapy for HER2-overexpressing ovarian cancer cells, an animal model of ovarian cancer was developed (Yu et al., 1995) that has since been used to demonstrate E1A-specific anti-tumor activity in vitro and in vivo using both liposomal (Yu et al., 1995) and adenovirus-mediated gene delivery systems. Similar anti-tumor activity by E1A was also shown in animal models of breast cancer (Chang et al., 1997). These results prompted the inventors to hypothesize that E1A functions as a tumor suppressor for HER2-overexpressing breast and ovarian cancer cells by repressing HER2 overexpression. On the other hand, mice with xenografts of a low-HER2-expressing ovarian cancer cell line did not respond to E1A (Zhang et al., 1995). E1A treatment also did not affect xenografts of a low-HER2-expressing breast cancer cell line or of stable transfectants of these cells made to constitutively express E1A; rather, mice in those 2 groups had similar survival rates. Thus, the inventors and others initially believed that E1A may not be effective for low-HER2-expressing breast or ovarian cancer, and efforts were focused on an ensuing phase I clinical trial on HER2-overexpressing breast and ovarian cancer.

That phase I study, conducted by the inventors and others in 1995, included safety and dose-finding studies of E1A, delivered locally with a DC-Chol cationic liposome (DCC) that had been shown to have good local transfection efficiency and minimal toxicity (Xing et al., 1997; Xing et al., 1998). Results from that trial showed that the E1A gene, complexed with cationic liposome, led to transfection of breast or ovarian cancer cells and that E1A expression resulted in HER2 downregulation (Hortobagyi and Ueno, 2001). Surprisingly, the present inventors found significantly reduced proliferation and increased induction of apoptosis regardless of HER2 expression level (Hortobagyi et al., 2001). In addition to its transcriptional repression of HER2, the present inventors and others have explored potential biological explanations for reduced tumorigenicity by E1A, e.g., through induction of apoptosis by both p53-dependent and p53-independent mechanisms (Barcellus et al., 1996; Rao et al., 1992; Mymryk et al., 1994; Sabbatini et al., 1995; White, 1995; Teodoro et al., 1995; Debbas and White, 1993; Lowe and Ruley, 1993; Shao et al., 1997), inhibition of NF-κB activation (Shao et al., 1997), suppression of metastasis via downregulation of metalloproteinase, downregulation of E-cadherin expression, or increased cytolysis by cytotoxic T lymphocytes or natural killer cells via increased immunogenicity (Mymryk, 1996). E1A represses the transcription of proteases that are important in metastasis, such as type IV collagenase (Yu et al., 1992; Frisch et al., 1990), plasminogen activator (Young et al., 1989), and interstitial collagenase and urokinase (Frisch et al., 1990). E1A can also repress tumor progression markers such as calcyclin, vimentin, urokinase, and tissue-type plasminogen activators in the metastatic melanoma cell line BLM (van Groningen et al., 1996). E1A regulates the differentiation of some cell types, e.g., embryonal carcinoma F9 cells (Nelson et al., 1990), and converts some tumor cells to an epithelial-cell morphology (Frisch, 1994). E1A can sensitize cells to undergo apoptosis, as can tumor necrosis factor-alpha (TNF-α) (Cook et al., 1989; Rodrigues et al., 1990; Shisler et al., 1996) and alkylating agents such as cisplatin and etoposide (Lowe and Ruley, 1993; Frisch, 1995; Sanchez-Prieto et al., 1996). Delivery of E1A sensitized HER2-overexpressing breast and ovarian cancer cells to paclitaxel, and treating mice bearing breast and ovarian cancer cell implants with paclitaxel and E1A led to prolonged survival compared with either treatment given alone; this improvement in survival was due to induction of apoptosis by downregulation of HER2 (Ueno et al., 2000; Araujo et al., 1993). Aside from. that work, findings reported over the past decade—suppression of tumorigenicity independent of HER2, inhibition of metastasis, and induction of apoptosis—have not been investigated in detail or developed as therapeutic modalities for the management of cancer. Nor has the possibility been addressed that E1A suppresses tumor formation by suppressing tumor-cell proliferation. Identifying such mechanisms regarding a reduction in proliferation may improve the efficacy of E1A gene therapy as well as identify new molecular targets for cancer treatment, such as breast and ovarian cancer treatment.

II. PEA15 is a Multifunctional Protein

Associated with E1A-mediated suppression of tumorigenicity of HER2 cells is PEA 15, which is an acidic, serine-phosphorylated, 15-kDa major phosphoprotein, is an endogenous substrate for protein kinase C (ser104), is associated with microtubules, and is highly enriched in astrocytes (Araujo et al., 1993). This phosphoprotein exists in vivo as three isoforms: the unphosphorylated form “N” and two phosphorylated forms, “mPa mono” (in which one site is phosphorylated) and “Pb” (in which two sites are phosphorylated). Pb has the highest affinity for microtubules and is found in membrane and microtubule fractions. Interactions between PEA15 and tubulins are regulated by phosphorylation. PEA15 colocalizes with α-tubulin even after treatment with paclitaxel (Danziger et al., 1995).

PEA15 seems to have an important role in astrocyte maturation, since both protein levels and phosphorylation levels increase during ontogenesis and are maximally expressed in the adult brain, particularly in the hippocampus. PEA15 is phylogenetically conserved in the brains of mammals, birds, and fish (Danziger et al., 1995). In astrocytes, PEA15 is phosphorylated by calcium-calmodulin-dependent protein kinase II (CAMKII, or ser116) and by protein kinase C (Kubes et al., 1998). PEA15 is encoded by two mRNAs, the full lengths of which are conserved in mice and in humans. Two cDNAs of the 3′-untranslated region (3′UTR) have been cloned from a mouse astrocyte library. The 136-amino acid protein sequence showed no sequence homology to any other protein but showed a 96% similarity to its human counterpart. The two mRNAs for PEA15 are expressed in a wide range of peripheral tissues, including lung, thymus, adrenals, heart, kidney, spleen and muscle, in addition to the brain. A counterpart of PEA15, PED (phosphoprotein-enriched in diabetes) controls glucose transport and is overexpressed in patients with type II diabetes mellitus. PED increases the levels of Glut1 transporters on the cell membrane and inhibits insulin-stimulated glucose transport by inhibiting cell surface recruitment of Glut4 (Condorelli et al., 1998).

When PED is overexpressed in MCF-7 and HeLa cells, the apoptotic pathway activated by exposure of cells to Fas ligand (FasL) and TNF-α is inhibited. This PED contains a death effector domain (DED) spanning more than 60% of the PED sequence. The N-terminal region of PED, which contains a DED, is highly homologous to that of FADD and FLICE, two proteins that are important in conveying FasL and TNF-α death signals into the cells. Specifically, PED associates with the DED of FADD and FLICE to displace FLICE binding to FADD, resulting in decreased activation of FLICE substrates in response to FasL and TNF-α. The overall effect is to inhibit the assembly of the receptor-FADD-FLICE signaling complex and abrogate activation of caspases (Condorelli et al., 1999). Studies of PEA15-null mice showed that PEA15 expression increases astrocyte survival after exposure to TNF (Kitsberg et al., 1999). However, in another study, PEA15 was shown to have a novel dual role-it inhibited Fas but increased the TNF-R1-mediated activity of caspase 8 and increased apoptosis. Therefore, the role of PEA15/PED in apoptosis is not well defined.

In contrast, 293 cells that overexpress PED show inhibited proliferation (Condorelli et al., 2002). Further, astrocytes containing high levels of PEA15 can proliferate, although more slowly than PEA15-replete cells (Formstecher et al., 2001). The ERK 1/2 MAP kinase pathway is known to control cell growth and survival and modulate integrin function. PEA15 blocks ERK-dependent transcription and proliferation by binding ERK in the cytoplasm and preventing its localization to the nucleus. PEA15 blocks the ability of ERK MAP kinase to phosphorylate the transcription factor Elk-1, thereby blocking ERK-dependent transcription (Formstecher et al., 2001; Hill et al., 2002) (FIG. 1). PEA15 has a nuclear export sequence that anchors ERK in the cytoplasm. Therefore, genetic deletion of PEA15 leads to increased localization of ERK in the nucleus, with subsequent increases in cFos transcription and cell proliferation. These findings indicate that PEA15 levels control the biological outcome of the ERK MAP kinase pathway by regulating the localization of ERK; also, the effect of MAT1 on PEA 15 expression levels may contribute to its transforming activity.

U.S. 20030049702 is directed to novel death domain proteins, including one having sequence homology with the death effector domain of PEA15. In a specific embodiment, these novel proteins modulate cellular processes such as apoptosis and cellular proliferation.

U.S. 20030215835 teaches treatment of prostate cancer by administering a therapeutic agent for regulating expression of a vast number of genes, including PEA15.

U.S. 20030134283 regards a combination of a plurality of cDNAs from dendritic cells or their complements, one of which is PEA15, and in a specific embodiment a molecule that regulates the activity of the cDNA or encoded protein is useful as a therapeutic for cancer.

WO2004108961 describes antisense PEA15 for the treatment of papillomas, diabetes, and breast cancer for example.

WO0240680 and US2003/0049702 describe death domains proteins, wherein PEA15 is disclosed in a list of same, as being useful in cancers such as gliomas, carcinomas, and so forth. In particular embodiments, the proteins are useful for diagnosis of cervical cancer, such as being associated with Chlamydia infection.

WO2003074007 concerns modulating lymphocyte activation with particular proteins, one of which is PEA15. T cell and B cell cancers are also treated with PEA15

III. Role of ERK in the MAP Kinase Pathway

MAP kinases play central roles in the growth, development, and survival of all eukaryotic organisms, including cancer cells. In mammalian cells, three major MAP kinase pathways have been characterized that result in activation of p42/44 or extracellular regulated kinases (ERK), p38/SAPK2, and Jun kinases (JNK) (Lewis et al., 1998; Cobb, 1999). Activation of MAP kinases by extracellular growth factors such as platelet-derived growth factor (PDGF) or HER2 autophosphorylation is regulated through control of the initiation and propagation of cascades of kinases. In addition, MAP kinases are dephosphorylated and inactivated by multiple phosphatases, including dual-specificity MAP kinase phosphatases (Waskiewicz and Cooper, 1995). The efficiency of MAP kinase activation is affected by scaffolding proteins that assemble the interacting components of the MAP kinase cascades (Schaeffer and Weber, 1999; Schaeffer et al., 1998; Whitmarsh and Davis, 1998; Burack and Shaw, 2000). MAP kinases elicit their biological outputs by phosphorylating nuclear and cytoplasmic substrates. Eventually, this is followed by the sequential recruitment and activation of Raf, MEK, and ERK. MEK binds and restricts inactive ERK to the cytosol (Fukuda et al., 1997). The MEK-ERK complex dissociates when MEK is activated and phosphorylates ERK. The ERK can then dimerize, and this dimerization is apparently required for ERK to translocate into the nucleus by active transport. This active (phosphorylated) form of ERK stimulates cell cycling (Malumbres and Barbacid, 2001; Downward, 2003) and enhances survival (Xia et al., 1995; Gupta et al., 1999). Cancer cells tend to show high activity in MAPK pathways, making these pathways a favorable target for the development of anti-cancer therapeutic agents.

IV. Systemic Gene Delivery with the LPD Liposome

As described above, the E1A gene was delivered by lipoplex, a cationic DC-Chol:DOPE liposome (DCC)-based delivery system. This non-viral delivery system, also used in preclinical animal studies, was selected as the gene delivery system for the clinical trial because of its biodegradability, its non-mutagenicity, and its low immunogenicity (Xing et al., 1997; Xing et al., 1998; Nabel et al., 1992; Nabel et al., 1993). In that trial, patients with breast or ovarian cancer were given the DCC-E1A liposome complexes by injection into the thoracic or peritoneal cavity. The results demonstrated successful transfection and expression of E1A, decreased expression of the HER2 oncogene, and the presence of apoptotic cells within the tumors (Hortobagyi et al., 2001). Although these results are encouraging, gene delivery by such lipoplex systems has thus far been limited to intratumoral or regional intracavitary delivery (Hortobagyi et al., 2001; Nabel et al., 1993; Yoo et al., 2001). Systemic gene delivery using DCC-E1A liposomes may be of limited effectiveness because of the inherent sensitivity of these complexes to serum, which may reduce transfection efficiency (Li and Huang, 1997).

A new lipid formulation based on a lipopolyplex, LPD, that demonstrated improved stability and enhanced transfection efficiency (Li and Huang, 1997; Li et al., 1998; Li et al., 1999; Nikitin et al., 1999; Tan et al., 1999; Whitmore et al., 1999), is an attractive candidate for the systemic delivery of therapeutic genes for advanced or metastatic cancer (Li and Huang, 1997; Li et al., 1998; Li et al., 1999; Tan et al., 1999; Whitmore et al., 1999). LPD is a cationic liposome/DNA complex comprised of a DOTAP:cholesterol liposome, protamine sulfate, and plasmid DNA. Unlike conventional cationic liposome/DNA complexes, which are larger and more heterogeneous in size, LPDs are small, condensed particles (average diameter, 180 nm) that contain a core of protamine-condensed DNA coated with a lipid bilayer. In immunocompetent mice, intravenous administration of LPD led to expression of its carried gene in normal organs, including the lung, heart, kidney, liver, and spleen, with the highest expression occurring in the lung (Li and Huang, 1997; Li et al., 1998).

V. Molecular Mechanisms of Paclitaxel

Paclitaxel is a taxane in common use for the treatment of breast and ovarian cancer. Paclitaxel produces mitotic arrest at the mitotic checkpoint by stabilizing the microtubules (Schiff et al., 1980; horwitz, 1994), which leads eventually to cell death by apoptosis. The anti-tumor effects of Paclitaxel seem to result from several mechanisms, including phosphorylation of Bcl-2 (Ling et al., 1998; Tang et al., 1994; Blagosklonny et al., 1996; Scatena et al., 1998), phosphorylation of Raf-1, inhibition of tumor angiogenesis (Klauber et al., 1997; Belotti et al., 1996), enhancement of c-jun N-terminal kinase (Wang et al., 1999), and increased expression of TNF-α (Burkhart et al., 1994). Whether these are secondary or primary effects remains to be seen. Paclitaxel also stimulates the MAPK pathway (Klauber et al., 1997; Belotti et al., 1996; Wang et al., 1999; Lieu et al., 1998; Shtil et al., 1999; Wang et al., 1998; McDaid et al., 2001; MacKeigan et al., 2000), which is a seemingly paradoxical phenomenon in that it can stimulate cell survival (Wang et al., 1999; Lieu et al., 1998; Shtil et al., 1999; Wang et al., 1998; McDaid et al., 2001; MacKeigan et al., 2000). Although investigators have attempted to inhibit MAPK to enhance paclitaxel sensitivity (McDaid et al., 2001; MacKeigan et al., 2000), other useful compositions for this purpose are desirable.

Thus, although a multitude of interrelated biological pathways are known to be associated with cancer, the present invention provides a useful and novel link among those described above, particularly regarding PEA 15 for anti-cancer activity.

### SUMMARY OF THE INVENTION

The present invention is generally directed to the modulation of cellular proliferation, particularly for the treatment of cancer, by introducing PEA15 (in specific embodiments may also be referred to as PED, MAT1, HMAT1, MAT1H, PEA-15, HUMMAT1H) to at least one cancerous cell. As described herein, the inventors identified PEA15 as a target gene of E1A. The adenovirus-type 5 gene early region 1A (E1A) is known to suppress the tumorigenicity of a variety of cancers (Ueno et al., 2001; Deng et al., 2002; Frisch and Mymryk, 2002). In a particular embodiment, however, E1A suppresses HER2-overexpressing cancer cells by downregulating their expression of HER2, yet also suppresses the tumorigenicity of some low-HER2-expressing cancer cells.

The precise mechanism by which E1A acts as a tumor suppressor in low-HER-2 expressing cells was heretofore not well defined. The present inventors demonstrate that PEA 15 binds to ERK and blocks ERK-dependent transcription and proliferation by sequestering ERK in the cytoplasm, preventing its localization in the nucleus. When the present inventors knocked down PEA15 expression in the OVCAR-3-E1A cells by siRNA, phospho-ERK accumulated in the nucleus. This indicates that sequestration of phospho-ERK in cytoplasm blocks ERK phosphorylation of nuclear substrates, which is necessary for tumorigenicity, in some embodiments of the present invention. The present inventors also demonstrated that PEA15 suppressed colony formation in the exemplary breast cancer cell lines MDA-MB-231 and MDA-MB-435. Given at least that PEA15 can sequester ERK, and that ERK is involved in cell proliferation, differentiation, and survival, in a specific embodiment of the present invention PEA 15 functions as a tumor suppressor gene.

Thus, in one particular aspect of the present invention, PEA15 is administered to a cancer cell. The cancer cell may be a tumor cell of an individual, such as a human individual, and although any cancer cell may be administered PEA15, in some exemplary embodiments the tumor is in the breast, the ovary, or tumors are present in both breast and ovary. PEA15 is delivered to a cancer cell in an individual to ameliorate at least one symptom of cancer in the individual and/or increase survival of the individual. Wherein the cancer cell is in a tumor, PEA 15 delivery may decrease the tumor size.

In particular aspects of the invention, the PEA15 delivery to the cancer cell facilitates or results in particular molecular characteristics in the cell. For example, the cancer cell to which PEA15 is being delivered may comprise E1A anti-tumor activity, such as through E1A-mediated induction of apoptosis and/or inhibition of angiogenesis. In another embodiment, such E1A-mediated activity or processes may be facilitated or enhanced following PEA15 delivery to the cell. In a further specific embodiment, the cancer cell to which PEA15 is being delivered may further comprise inhibition of ERK-dependent transcription and/or proliferation, and/or such ERK-dependent transcription and/or proliferation may be facilitated or enhanced following PEA15 delivery to the cell. In yet another specific embodiment, the cancer cell to which PEA15 is delivered may further comprise sequestration of ERK to the cytoplasm, and/or such sequestration of ERK to the cytoplasm may be facilitated following PEA 15 delivery to the cell.

In one aspect of the invention, at least the aforementioned molecular characteristics are interrelated in one or more pathways comprising at least one PEA15 activity, such as tumor suppressor activity. For example, E1A anti-tumor activity may be provided to or present in a cancer cell, and delivery of PEA15, being downstream in a pathway comprising E1A, upregulates at least part of the pathway. Upon delivery of PEA15, there is inhibition of ERK-dependent transcription and/or proliferation, such as by PEA15-mediated (either directly or indirectly) sequestration of ERK in the cytoplasm. The absence of ERK in the nucleus prevents it from stimulating cell cycling and enhancing survival of the cell. Thus, PEA15 is an effective anti-cancer therapeutic agent, in preferred embodiments of the invention.

In some aspects of the invention, tumor suppressor activity may comprise transcriptional repression of an oncogene, conversion of tumor cells to an epithelial phenotype, suppression of metastatic potential, induction of apoptosis, increasing sensitivity to tumor necrosis factor and/or reduction of cell proliferation, such as through reduction of cell growth or rate of growth.

Thus, the present invention generally relates to methods and compositions related to PEA15 as a tumor suppressor. In a particular aspect of the invention, PEA15 is utilized for repressing or preventing transformation in and/or proliferation of a cell, the method comprising contacting the cell with PEA15 in an amount effective to inhibit a transformed phenotype and/or reduce proliferation of the cell. The source organism of the PEA15 may be from any organism, so long as it provides anti-cancer activity, such as through tumor suppressor activity. Inhibition of transformation may be indicated by a reduction in a transforming, tumorigenic or metastatic potential of a cell. Such cells may be in cell culture. More preferably, the cells in which transformation is repressed are cells in a living organism, for example a human. The inhibition of such transformation has great utility in the prevention and treatment of such transformation-driven events such as cancer, tumorigenesis, and metastasis.

Herein, the terms “PEA15 gene product” and “PEA15” refer to proteins having amino acid sequences that are substantially identical to human PEA15 (hPEA15) or murine PEA15 (mPEA15), for example, or that are biologically active in that they are capable of cross-reacting with anti-PEA15 antibody raised against PEA15, or which are biologically active in that they are capable of exhibiting tumor suppressor activity. “PEA15 gene product” and “PEA15” refer to proteins, polypeptides, or peptides having amino acid sequences that are substantially identical to human PEA15 (hPEA15) or murine PEA15 (mPEA15) amino acid sequence, for example, and that are biologically active in that they are capable of cross-reacting with anti-hPEA15 or anti-mPEA15 antibodies raised against hPEA15 or mPEA15, respectively, and/or capable of tumor suppressor activity. “PEA15 gene product” also includes analogs of PEA15 molecules that exhibit at least some biological activity in common with PEA15. In an alternative embodiment, the PEA 15 gene product is a catalytic RNA.

Herein, the term “PEA15 gene,” which may also be referred to as “PEA15 polynucleotide,” refers to any DNA sequence that is substantially identical to a DNA sequence encoding a PEA15 gene product as defined above. The term also refers to RNA, or antisense sequences compatible with such DNA sequences. A “PEA15 gene” may also comprise any combination of associated control sequences. A “PEA15 gene” may also be referred to herein as a “PEA15 polynucleotide”.

As used in this specification and the appended claims, the singular forms “a” “an” and “the” generally mean “at least one”, “one or more”, and other plural references unless the context clearly dictates otherwise. Thus, for example, references to “a cell”, “a polypeptide” and “a sequence” include mixtures of cells, one or more polypeptides and a plurality of sequences of the type described; and reference to “PEA15” includes different species of such PEA15 and so forth.

PEA15 is a polypeptide that may be contacted with or introduced to a cell through any of a variety of manners known to those of skill. The PEA15 polypeptide may be introduced through direct introduction of a PEA polypeptide to a cell, such as a human PEA polypeptide. In this case, the PEA15 polypeptide may be obtained through any method known in the art, although it is anticipated that in vitro expression of the PEA15 polypeptide in a cell culture system may be a preferred manner of obtaining PEA15.

In one aspect of the invention, there is a pharmaceutical formulation comprising a PEA15 peptide, polypeptide, or both in a pharmaceutically acceptable carrier, wherein the PEA15 peptide or polypeptide comprises SEQ ID NO:19 or a mutant thereof that comprises a mutation at one or more of the following residues: Ser25, Ser104, or Ser116.

In particular aspects of the invention, the PEA15 of the compositions and methods of the invention may comprise an alteration in the PEA15 gene product. A polynucleotide encoding a PEA15 gene product comprising an alteration is encompassed in the invention. In specific embodiments, one or more alterations may be in any position in the PEA15 gene product, so long as the PEA15 comprises activity to inhibit proliferation of a cancer cell and/or inhibits growth of a tumor, for example, such as tumor suppressor activity. In specific embodiments, the alteration is at a phosphorylation site, more than one phosphorylation site, a death effector domain, a protein-protein interaction domain, another domain, or a combination thereof. In further specific embodiments, the amino acid that is altered is Ser25, Ser104, Ser116, or a combination thereof.

PEA15 may also be introduced to a cell via the introduction of a nucleic acid, which may also be referred to as a polynucleotide, that encodes the PEA15 polypeptide in the cell. For example, RNA or DNA encoding PEA15 may be introduced to the cell by any manner known in the art.

In certain preferred embodiments, the PEA15 is introduced into the cell through the introduction of a DNA segment that encodes PEA15. In some such embodiments it is envisioned that the DNA segments will further comprise the PEA15 gene operatively linked to its associated control sequences. For example, the PEA15 gene may be operatively linked to a suitable promoter and a suitable terminator sequence. The construction of such gene/control sequence DNA constructs is well-known within the art. In a specific embodiment, the promoter is active in the tissue to which PEA15 is being introduced. For cancer treatment, the promoter is preferably operable in the tissue comprising the cancer (which may be a solid tumor, or in alternative embodiments is not in a solid tumor). For example, for the exemplary breast cancer or ovarian cancer therapy, a promoter active in breast tissue or ovarian tissue, respectively, is desirable. Examples of breast tissue-specific control regions, such as promoters, include tissue-specific promoters such as, for example, Muc-1, CEA, PSA, HER-2, Myc, L-plastin and secretory leukoproteinase inhibitor promoters (for review, see Patterson and Harris, 1999). Examples of ovarian-specific control include the tissue-specific SLPI promoter and the ovarian cancer associated targeting adaptor protein, sCARfC6.5 (Barker et al., 2003); the vitellogenin promoter (Tsang et al., 2003), the secretory leukoprotease inhibitor (SLPI) promoter (Barker et al., 2003) and others, such as are listed in the review by Wolf and Jenkins (2002).

In certain embodiments for introduction, the DNA segment may be located on a vector, for example, a plasmid vector or a viral vector. The viral vector may be, for example, a retroviral vector, an adenoviral vector, or an adeno-associated viral vector. Such a DNA segment may be used in a variety of methods related to the invention. The vector may be used to deliver a PEA 15 gene to a cell in one of the gene-therapy embodiments of the invention. Also, such vectors can be used to transform cultured cells, and such cultured cells could be used, inter alia, for the expression of PEA 15 in vitro.

In certain aspects of the invention, the PEA15 polypeptide or PEA15-encoding nucleic acid is complexed with a liposome for introduction to a cell. In some embodiments, the liposome comprises one or more of DOTMA, DOPE, DC-Chol, or LPD. In some specific embodiments, the liposome comprises LPD, and the vector comprises a polynucleotide encoding PEA15 in a plasmid.

In particular embodiments the PEA15 is introduced into a cell that is a human cell. In many embodiments the cell is a cancerous cell. In certain exemplary embodiments the cancerous cell is a breast cancer cell or an ovarian cancer cell. However, any kind of cancer cell may be treated, including at least breast cancer cell, ovarian cancer cell, endometrial cancer cell, prostate cancer cell, livery cancer cell, glioblastoma cell, head and neck cancer cell, bladder cancer cell, pancreatic cancer cell, colon cancer cell, lung cancer cell, thyroid cancer cell, or brain cancer cell, for example. The cancerous cell may be in a tumor, although in an alternative embodiment it is not in a tumor.

The present invention further provides methods to suppress the growth of an oncogene-mediated tumor in a mammal, the method comprising administering to said tumor a composition comprising PEA15, wherein said administration results in a decrease in the growth rate of said tumor. In some preferred embodiments, there is introduction of a nucleic acid encoding PEA15 operatively linked to a promoter wherein the production of the PEA15 results in a decrease in the growth rate of said tumor. In some preferred aspects of the present invention, the PEA15 polypeptide or encoding nucleic acid is administered in a liposomal complex.

The PEA15 gene products and nucleic acids of the present invention may also be introduced using any suitable method. A “suitable method” of introduction is one that places a PEA 15 gene product in a position to inhibit the transformation or reduce proliferation of a cell. For example, injection, oral, and inhalation methods may be employed, with the skilled artisan being able to determine an appropriate method of introduction for a given circumstance. In some preferred embodiments, injection will be used. This injection may be intravenous, intraperitoneal, intramuscular, subcutaneous, intratumoral, intrapleural, or of any other appropriate form.

In certain other aspects of the present invention there are provided therapeutic kits comprising in at least one suitable container a pharmaceutical formulation of a PEA15 gene product or a nucleic acid encoding a PEA15 gene product. Such a kit may further comprise a pharmaceutical formulation of a therapeutic polypeptide, nucleic acid encoding a therapeutic polypeptide, or chemotherapeutic agent. The kit may also comprise a delivery vehicle for the PEA15 gene product or PEA15-encoding polynucleotide, such as a liposome, for example LPD.

In some embodiments of the present invention, the discovery that PEA15 is able to inhibit transformation and/or suppress tumors is used in combination with other anti-transformation/anti-cancer therapies. These other therapies may be known at the time of this application or may become apparent after the date of this application. PEA15 may be used in combination with other therapeutic polypeptides, nucleic acid encoding other therapeutic polypeptides, or chemotherapeutic agents, for example. That is, PEA15 may be used in conjunction with other known anti-cancer polypeptides, such as p53. PEA15 may be used in conjunction with any known transformation or disease inhibitor. PEA 15 may be used with other gene-therapy regimes. PEA15 may be used with any suitable chemotherapeutic drug.

In an embodiment of the present invention, there is a pharmaceutical formulation comprising PEA15-encoding nucleic acid operatively linked to control sequences, said formulation in a pharmaceutically acceptable carrier. The PEA15-encoding nucleic acid may be complexed with a liposome, such as one that comprises DOTMA, DOPE, DC-Chol, or LPD, for example. In additional embodiments, the pharmaceutical formulation further comprises a second agent, wherein the second agent is a therapeutic polypeptide, a nucleic acid encoding a therapeutic polypeptide, a chemotherapeutic agent, or a mixture thereof. In another specific embodiment, the PEA 15-encoding nucleic acid is comprised in a vector, such as a plasmid vector or a viral vector. The viral vector may be of any suitable kind, and some exemplary viral vectors include retroviral vectors, adenoviral vectors, adeno-associated viral vectors, lentiviral vectors, herpes viral vectors, vaccinia viral vectors, and so forth. In particular aspects of the invention, the control sequences comprise a heterologous control sequence.

In other embodiments of the invention, there is a pharmaceutical formulation comprising a PEA15 peptide, polypeptide, or both in a pharmaceutically acceptable carrier. A death effector domain of the PEA15 may comprise amino acids 1-79 of SEQ ID NO: 19, for example.

In an additional embodiment of the present invention, there is a method for repressing or inhibiting transformation in a cell, the method comprising contacting said cell with PEA15 in an amount effective to repress or inhibit a transformed phenotype. In a specific embodiment, the PEA15 is introduced into the cell through the introduction of a PEA15-encoding nucleic acid, which may further comprise control sequences operatively linked to the PEA15-encoding nucleic acid, for example The PEA15-encoding nucleic acid may be located on a vector, in specific embodiments, such as a plasmid vector or a viral vector, including a retroviral vector, for example. In a specific embodiment, a tumorigenic potential of the cell is suppressed. In another specific embodiment, a metastatic potential of the cell is suppressed. In a specific embodiment, a PEA15-encoding nucleic acid is complexed with a liposome, such as DOTMA, DOPE, DC-Chol, or LPD. The cell may be a human cell, such as a tumor cell, including a breast tumor cell or an ovarian tumor cell, for example.

In specific embodiments of the invention, the repression or inhibition of the transformed phenotype is further defined as comprising sequestration of ERK in the cytoplasm of the cell. In another specific embodiments, the tumorigenicity of the tumor cell results directly or indirectly from ERK-dependent transcription and proliferation.

Methods of the present invention may further comprise treating the cell with a second agent, wherein the second agent is a therapeutic polypeptide, nucleic acid encoding a therapeutic polypeptide, or chemotherapeutic agent.

In an additional embodiment of the invention, there is a method to suppress the growth of a tumor in a mammal, the method comprising introducing to the mammal a PEA15-encoding nucleic acid wherein expression of PEA15 in the mammal results in a decrease in the growth of said tumor. In a specific embodiment, the PEA15-encoding nucleic acid is introduced to the mammal via a vector. PEA15-encoding nucleic acids may be introduced into the mammal via a liposomal complex, such as one that comprises DOTMA, DOPE, DC-Chol, or LPD. In a specific embodiment, the tumorigenicity of the tumor results directly or indirectly from ERK-dependent transcription and proliferation. In another specific embodiment, the decrease in the growth of the tumor results directly or indirectly from sequestration of ERK in the cytoplasm of at least one cell of the tumor. In another embodiment there is a method of treating mammalian breast or ovarian cancer cells lacking functional wild-type PEA15 protein, comprising introducing a wild-type PEA15 tumor suppressor gene encoding said endogenous wild-type PEA15 protein of SEQ ID NO:19 or a mutant comprising a mutation at Ser25, Ser104, and/or Ser116 into said mammalian cancer cells.

In an additional embodiment, there is a method for suppressing the growth of a tumor in a mammal, the method comprising administering directly to the tumor a vector comprising a PEA15-encoding nucleic acid sequence encoding a PEA15 protein having tumor suppressor activity, the nucleic acid sequence operatively linked to a promoter, wherein production of the PEA15 protein results in a decrease in the growth of the tumor. The mammal may be a human and/or the tumor is in a breast or an ovary. In a specific embodiment, the method further comprises the step of subjecting the mammal to an additional cancer therapy, such as surgery, chemotherapy, radiation, immunotherapy, hormonal therapy, or a combination thereof, for example. In a specific embodiment, a tumor is a breast tumor, ovarian tumor, endometrial tumor, prostate tumor, liver tumor, glioblastoma tumor, head and neck tumor, bladder tumor, pancreatic tumor, colon tumor, lung tumor, thyroid tumor, prostate tumor, cervical tumor, or brain tumor.

In another embodiment of the invention, there is a method of treating mammalian cancer cells lacking functional wild-type PEA15 protein, comprising introducing a wild-type PEA15 tumor suppressor gene encoding said endogenous wild-type PEA15 protein into said mammalian cancer cells. 1

In an additional embodiment of the present invention, there is a method for facilitating or enhancing sensitivity of a cancer cell to a chemotherapeutic agent. In another words, there is a method of sensitizing a cancer cell to chemotherapy, both methods comprising the step of delivering to the cell a PEA15 polynucleotide or PEA15 polypeptide. Although the cell may need sensitization to any chemotherapy agent, in specific embodiments the cell is resistant to an ovarian or breast cancer chemotherapeutic agent. In an exemplary embodiment, the chemotherapeutic agent is paclitaxel, cyclophosphamide, cytotoxan, pamidronate/aredia, doxorubicin, adriamycin, tamoxifen, cisplatin, carboplatin, melphalan, or combinations thereof, for example.

In an additional embodiment of the present invention, there is a therapeutic kit comprising in at least one suitable container, a pharmaceutical formulation of a PEA15 gene product or a nucleic acid encoding a PEA15 gene product. In a specific embodiment, the kit further comprises a pharmaceutical formulation of a therapeutic polypeptide, nucleic acid encoding a therapeutic polypeptide, chemotherapeutic agent, or mixture thereof.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description. That is, the foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying exemplary drawings.

FIG. 1 illustrates PEA15 binding to ERK (PERK; active form), sequestering pERK in the cytoplasm. This results in inactivation of MAPK pathway, which may result in reduced proliferation and inhibited apoptosis (enhanced survival) or cancer cells.

FIGS. 2A through 2C show that HER2 downregulation is observed in a cell expressing E1A. E1A and HER2 immunohistochemical staining of breast cancer at 22 days after DCC-E1A complex administration is provided. In FIG. 2A, it shows before treatment (Day 0), 3+ HER2 staining. In FIG. 2B it shows after treatment (Day 22), 1+ HER2 staining. In FIG. 2C, it shows E1A expression in cytoplasm and nucleus.

FIG. 3 demonstrates that DCC-E1A suppressed the proliferation of tumor cells from patients with breast cancer.

FIGS. 4A through 4D show that E1A suppresses tumorigenicity in low HER2-expressing OVCAR3 (OV3) cells. In FIG. 4A, there are western blots showing the parental human ovarian cancer cell line OV3, OV3-NP (transfected with the vector pSV2-neo, which encodes only neomycin resistance gene), and OV3-E1A clones stably transfected with the E1A gene. In FIG. 4B, BrdU incorporation (yellow) shows reduced DNA synthesis in the OV3-E1A cell line. In FIG. 4C, anchorage-independent growth of the E1A transfectants shows that fewer colonies formed in soft agar (P<0.05). In FIG. 4D, inhibited colony formation by the OV3-E1A cells correlates with in vivo evidence of reduced tumorigenicity after E1A gene therapy (60% tumor-free survival) in a preclinical model of ovarian cancer in nude mice (P<0.01).

FIGS. 5A through 5B show that ERK accumulates in the nucleus of OV3-A1A cells with PEA15 knockdown. IN FIG. 5A, PEA15-siRNA knocks down PEA15 in OV3-E1A stable transfectants (bottom). PEA15 expression (green) is high in the control cells. In FIG. 5B, there is knockdown of PEA15 (green) that leads to accumulation of pERK (red) in the nucleus. After PEA15-siRNA treatment, OV3-E1A cells were double-stained for PEA15 and ERK. At top, ERK in the control cells is present in the nucleus and cytoplasm. At bottom, ERK in the PEA15-siRNA cells is mostly in the nucleus.

FIG. 6 shows that PEA15 represses colony formation of ovarian (SKOV3-ip1, OVCAR-3) and breast cancer (MDA-MB-231) cells. Cells were transfected with either pcDNA3HA-PEA15 or pcDNA3-neo, selected with medium containing the neomycin analogue G418, and stained with crystal violet. Colony formation was determined by comparing the numbers of colonies formed by pcDNA3HA-PEA15 cells with those formed by the pcDNA3 (control) cells. Colony formation was reduced in OVCAR-3 cells by 40%, in SKOV3ip1 cells by 50%, and in MDA-MB-231 cells by 50%.

FIG. 7 demonstrates that PEA15 represses colony count in the human ovarian cancer cell line SKOV3-ip1 cells. Aliquots of ip1 cells transfected with pcDNA3-neo (which encodes a neomycin resistance gene) and ip1-PEA-15 clones stably transfected with HA-PEA15 cell lines (2×10³) were mixed at 37° C. with 0.5% agarose (Low Melting Agarose; Fischer Scientific; Fairlawn, N.J.) in DMEM-F12 medium and gelled at 4° C. for 20 min over a previously gelled layer of 1% agarose in DMEM/F12 medium in triplicate in six-well dishes. After incubation for 3 weeks, 200 μL of 1 mg/mL p-iodonitrotetrazolium violet was added and incubated for an additional 24 h. Colonies were photographed with a Zeiss microscope and counted using software provided by Zeiss.

FIG. 8 shows that PEA15 increases apoptosis in the human ovarian cancer cell line SKOV3-ip1 cells. Aliquots of ip1 cells transfected with pcDNA3-neo (which encodes a neomycin resistance gene) and ip1-PEA15 clones stably transfected with HA-PEA15 cell lines (2×10³) were treated with 0.1 mM of paclitaxel. After incubation for 72 hrs, sub GO fractions by FACS were analyzed.

FIG. 9 provides immunohistochemical analysis of MDA-MB-361 tumor tissue from mice treated with LPD-E1A. Tumor-bearing mice were treated with i.v. LPD-E1A or LPD-luciferase (20 μg DNA), and tumor tissues were harvested 48 h later. Expression of E1A protein was associated with downregulation of HER2 protein expression and the presence of TUNEL-positive apoptotic cells. Top panels depict LPD-luciferase samples; bottom panels depict LPD-E1A treated samples.

FIG. 10 demonstrates LPD-E1A, given alone or in combination with paclitaxel, decreased tumor growth in HER2-overexpressing tumor xenografts. Female nude mice were inoculated with HER2-overexpressing MDA-MB-361 cells in the mammary fat pad. After the formation of 8-10 tumors per group (5 animals per group, -2 tumors per animal), mice were given one of the following treatments: naked E1A DNA (20 μg DNA, light circles), vehicle only (small squares), LPD-luciferase (20 μg DNA, triangles), LPD-E1A (20 μg DNA, dark circles), paclitaxel (15 mg/kg, light triangles), or LPD-E1A (20 μg DNA) plus paclitaxel (15 mg/kg) (large squares). The tumor growth ratio was determined by comparing the size of the treated tumors with the size of the original tumor before any treatment. The error bars indicate s.e.m.

FIGS. 11A-11D show that E1A inhibits proliferation of low-HER2-expressing ovarian cancer cells in vitro. In FIG. 11A, viability of OVCAR-3 cells after Ad.E1A(+) treatment is shown Cells (2×10⁵/well) were infected once with 10 MOI of Ad.E1A(+), and viable and dead cells were counted daily for 5 days by trypan blue staining. Numbers of viable Ad.E1A(+) cells dropped precipitously by day 3 (P<0.05 vs. cells infected with Ad.E1A(−) or control cells), with the proportion of dead cells peaking at 92.4% at 5 days. Results represent the means ±standard deviations from three independent experiments. B, E1A protein expression in the E1A stable transfectant clones OVCAR-E27 and OVCAR-E28 was confirmed by western blotting with an anti-E1A antibody. SKOV3.ip1-E1A cells were used as a positive control and OVCAR-NP as a negative control. C, DNA synthesis, measured by the amount of BrdU incorporated into the cells, was reduced in OVCAR-E1A cells. D, Tests of the anchorage-independent growth of OVCAR-3, OVCAR-NP and OVCAR-E1A cell lines showed that the OVCAR-E1A cells formed fewer colonies in soft agar than did the control cells.

FIGS. 12A-12C show upregulation of PEA15 results in sequestration of ERK in the cytoplasm in OVCAR-E1A cells. In FIG. 12A, western blot analysis showed upregulation of PEA15 protein expression in OVCAR-E1A cells relative to that in the parental and vector-transfected cells. In FIG. 12B, immunocytochemical analysis showed increased expression of PEA15 in the cytoplasm of OVCAR-E1A cells relative to that in the cytoplasm of the vector control OVCAR-NP cells. The nuclei were stained with propidium iodide, and PEA 15 was detected with FITC-conjugated rabbit antibody. In FIG. 12C, cellular localization of endogenous ERK and pERK in OVCAR-E1A cells and OVCAR-NP controls is shown. Equal amounts (50 μg) of cytoplasmic (C) and nuclear (N) fractions were analyzed by PAGE on a 12% SDS gel. The blots were probed with antibodies to ERK1/2 (top row), pERK (second row) and L-tubulin (third row) and PARP (bottom row) to verify separation of the cytosolic and nuclear fractions. Most of the ERK in the OVCAR-E1A cells accumulated in the cytoplasm rather than in the nucleus. Less ERK was expressed in the E1A-expressing cells than in the control cells.

FIGS. 13A-13D demonstrate that depletion of PEA15 leads to accumulation of pERK in the nuclei of OVCAR-E1A cells. In FIG. 13A, a 21-nt siRNA sequence targeting PEA15 reduced the expression of PEA15 (green) in OVCAR-E1A stable transfectants at 65 h after transfection, as confirmed in western blot analysis. In FIG. 13B, depletion of PEA15 led to accumulation of pERK in the nuclei of OVCAR-E1A cells. The nuclei were stained with DAPI (blue). The pERK (green) in the siRNA control-treated cells was in both nuclei and cytoplasm (top) but was mostly in the nuclei of the siPEA15-treated cells (bottom). In FIG. 13C, Elk-i transcriptional activity was decreased in OVCAR-E1A cells. OVCAR-NP and OVCAR-E1A cells were transfected with 1.2 μg of the indicated constructs [pFR-luc (1 μg), pFA2-Elk-1 (50 ng), and pRL-TK (100 ng)]. Cells were lysed 48 h later and Elk-1 transcriptional activity was measured. In FIG. 13D, knockdown of PEA15 expression by siRNA restored Elk-1 activity in OVCAR-E1A cells. Cells were transfected with siControl or siPEA15 and transfected again the next day with 1.2 μg of the indicated constructs [pFR-luc (1 μg), pFA2-Elk-1 (50 ng), and pRL-TK (100 ng)], grown in reduced serum (0.5%) for 24 h, and then stimulated with medium containing 20% serum for 30 min, after which cells were lysed and Elk-1 transcriptional activity measured.

FIGS. 14A-14B shows that depletion of PEA15 leads to increased proliferation of and colony formation by OVCAR-E1A cells. In FIG. 14A, to assess proliferation, OVCAR-E1A cells were untreated or transfected with siRNA control or siRNA against PEA15. At 30 h after transfection, cells were incubated with BrdU for 20 h, stained with anti-BrdU-FITC for 30 min, and then sorted by a BD FACScan flow cytometer to determine the percentages of proliferating cells. In FIG. 14B, soft agar colony formation was assessed by transfecting OVCAR-E1A cells with siRNA control or siRNA-PEA15 for 48 h, after which cells were plated, incubated for 3 weeks, stained with p-iodonitrotetrazolium violet, and colonies counted. The results shown here are representative of three independent assays, each done in triplicate.

FIGS. 15A-15D show PEA15 expression mediates antitumor activity in vitro in ovarian cancer and breast cancer cells. In FIG. 15A, PEA15 suppressed colony formation by OVCAR-3 and SKOV3.ip1 human ovarian cancer cells and by MDA-MB-231 human breast cancer cells. Cells were transfected in 100-mm plates with either pcDNA3HA-PEA15 or pcDNA3, selected with G418, and stained with crystal violet. Colony formation was quantified by comparing the number of G418-resistant colonies of HA-PEA15-transfected cells with the number of colonies of pcDNA3-transfected cells. In FIG. 15B, PEA 15 protein expression in the three SKOV3.ip1-PEA15 clones (ip1-P1, -P2, -P3) was confirmed by western blotting with an anti-HA antibody; a neomycin pool was used as a negative control. In FIG. 15C, PEA15 inhibited the growth rate of clones 1 and 2 but not that of the low-PEA15-expressing clone 3. Data represent the means from four independent experiments. In FIG. 15D, suppression of colony formation by PEA15. Parental SKOV3.ip1 cells, vector controls, and three PEA15 stable transfectants were grown in soft agar for 3 weeks and stained with p-iodonitrotetrazolium. Percentages are relative to the vector-only-transfected cells, which were scored as 100%.

FIG. 16 shows that an Ad.PEA15 composition inhibits growth in some breast and ovarian cancer cells (MTT).

FIG. 17 shows that an Ad.PEA15 composition inhibits growth in MDA-MB-468 cells (trypan blue).

FIG. 18 demonstrates that PEA15 inhibits growth in MCF-7 cells (trypan blue).

FIG. 19 provides an exemplary cell growth curve in MDA-468 and OVCAR-3 cells treated with PEA-15 compared to a control.

FIG. 20 shows cell viability in MDA-468 and OVCAR-3 cells treated with PEA15 compared to a control.

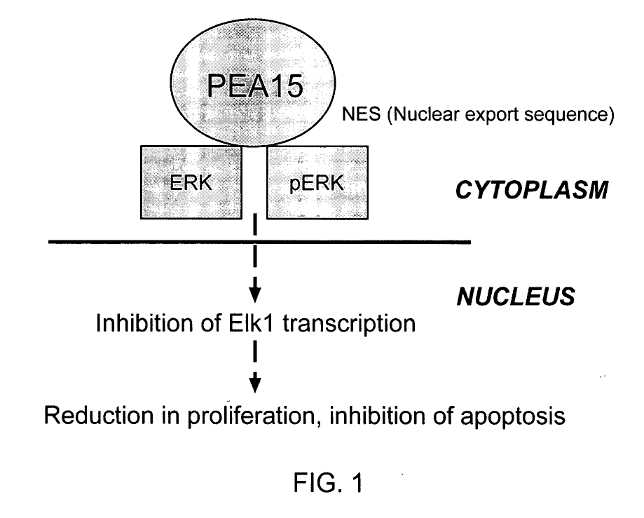

### DETAILED DESCRIPTION OF THE INVENTION

Cancer is a major cause of death in both men and women. In particular, breast and ovarian cancer are major causes of cancer death among women. Overexpression of the HER-2/neu oncogene has been reported to correlate with poor survival in patients with breast or ovarian cancer, to enhance the metastatic potential of human cancer cells, and to induce resistance to certain chemotherapeutic agents such as paclitaxel (which may also be referred to as Taxol). Therefore, HER2 has been a target for development of novel anticancer agents for HER2-overexpressing cancer cells. The disclosure provided herein indicates that adenovirus type 5 E1A, a transcriptional modulator, represses HER2 transcription in HER2-overexpressing cancer cells, and local administration of an E1A-liposome complex can inhibit tumor development in an animal model of the exemplary breast and ovarian cancer. The present inventors also showed in a phase I clinical trial that E1A can also sensitize breast and ovarian cancer cells to paclitaxel by downregulating HER2 expression.

In the present inventors' phase I study of E1A gene therapy, transfection of breast or ovarian cancer cells with E1A led to reduced proliferation and induction of apoptosis of those cells, regardless of their HER2 expression. More recent results showed that E1A can suppress the tumorigenicity of low HER2-expressing cancer cells, which indicates that several molecular mechanisms are responsible for E1A anti-tumor activity.

The disclosure provided herein shows that upregulation of PEA15 by E1A is one such mechanism, in specific embodiments. In a specific embodiment, E1A suppresses tumorigenicity by upregulating PEA15. As shown herein, PEA15 suppresses colony formation by the exemplary breast cancer and ovarian cancer cell lines. PEA15 is also known to sequester ERK in the cytoplasm. This ERK (MAPK) pathway is known to promote cell proliferation, differentiation, and survival. Therefore, preventing the phosphorylation of ERK's nuclear substrate, Elk, may lead to reductions in the proliferation and survival of cancer cells.

Furthermore, paclitaxel activates the prosurvival MEK/ERK pathway. In a specific embodiment of the invention, because activated ERK can be sequestered by PEA15, PEA15 then sensitizes cancer cells to paclitaxel by inactivating the ERK pathway. In one aspect of the invention, combining paclitaxel and PEA15 enhances apoptosis in cancer cells, in some embodiments through a synergistic effect in which ERK, activated by paclitaxel, is bound to PEA15, which would prevent the active form of ERK from being localized in the nucleus and hence suppress its activity. The combination of paclitaxel with PEA15 is a novel molecular approach to a combined strategy, such as a combined strategy comprising chemotherapy, gene therapy, and/or radiation, for example.

Parallel studies conducted by other investigators showed that E1A could also induce apoptosis and inhibit angiogenesis independent of HER2 downregulation. Indeed, the present inventors determined that proliferation was decreased and apoptosis was increased in low HER2-expressing breast or ovarian cancer cells from patients who had undergone E1A gene therapy. The precise mechanism by which E1A suppresses proliferation and increases apoptosis in low HER2-expressing cancer is not well defined.

To address this issue, the present inventors modified the low HER2-expressing human ovarian cancer cell line OVCAR3 (OV3) to constitutively express E1A (hereafter referred to as OV3-E1A cells) and compared the biological characteristics of the OV3-E1A cells and the parental cells. Cell proliferation, anchorage-independent growth in soft agar, and tumorigenicity were all suppressed in OV3-E1A cells as compared with the parental cells. To identify the molecular mechanism underlying the reduction in anchorage-independent growth, the present inventors compared the gene expression profiles of OV3-E1A and OV3 cells and found upregulation of PEA15, indicating that PEA15 is a target gene of E1A. PEA15 binds to the MAP kinase ERK and blocks ERK-dependent cell proliferation by sequestering ERK in the cytoplasm and preventing its localization in the nucleus, where it is known to activate target genes related to cell proliferation, survival, and differentiation. In a specific embodiment of the present invention, PEA15, upregulated by E1A, inhibits tumorigenicity by blocking ERK-dependent cell proliferation. In a specific embodiment of the present invention, PEA15, upregulated by E1A, functions as a tumor suppressor gene. In a particular aspect of the invention, PEA15 comprises an anti-tumor therapeutic agent for the treatment of cancer, such as breast and ovarian cancer. In another particular aspect of the invention, novel cationic liposomes to deliver PEA15 locally (DC-Chol) and systemically (LPD) are utilized in compositions and methods herein.

I. Definitions and Techniques Affecting Gene Products and Genes

A. PEA15 Gene Products and Genes

Herein the terms “PEA15 gene product” and “PEA15” refer to proteins having amino acid sequences that are substantially identical to the human PEA15 (hPEA15) or murine PEA15 (mPEA15), for example, or which are biologically active in that they are capable of cross-reacting with anti-PEA15 antibody raised against PEA15, or which are biologically active in that they comprise tumor suppressor activity. “PEA15 gene product” and “PEA15” refer to proteins having amino acid sequences that are substantially identical to the human PEA15 (hPEA15) or murine PEA15 (mPEA15) amino acid sequence, for example, and that are biologically active in that they are capable of binding to ERK and/or cross-reacting with anti-hPEA15 or anti-mPEA15 antibodies raised against hPEA15 or mPEA15, respectively. The term “PEA15 gene product” also includes analogs of hPEA15 or mPEA15 molecules, for example, that exhibit at least some biological activity in common with HPEA15 or mPEA15, respectively. Furthermore, those skilled in the art of mutagenesis will appreciate that other analogs, as yet undisclosed or undiscovered, may be used to construct PEA15 compositions.

The term “PEA15 gene” or “PEA15 polynucleotide” refers to any DNA sequence that is substantially identical to a DNA sequence encoding an hPEA15 or mPEA15 gene product, for example, as defined above. The term also refers to RNA, or antisense sequences compatible with such DNA sequences. A “PEA15 gene” may also comprise any combination of associated control sequences.

The term “substantially identical”, when used to define either a PEA15 amino acid sequence or PEA15 gene nucleic acid sequence, means that a particular subject sequence, for example, a mutant sequence, varies from the sequence of natural PEA 15 by one or more substitutions, deletions, or additions, the net effect of which is to retain at least some biological activity of the PEA15 protein. Alternatively, DNA analog sequences are “substantially identical” to specific DNA sequences disclosed herein if: (a) the DNA analog sequence is derived from coding regions of the natural PEA15 gene; or (b) the DNA analog sequence is capable of hybridization of DNA sequences of (a) under moderately stringent conditions and which encode biologically active PEA15; or (c) DNA sequences which are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequence.

Exemplary PEA15 polynucleotides (referred to by their number from the National Center for Biotechnology Information's GenBank database) include the following: AF153273 (SEQ ID NO:1); AF153274 (SEQ ID NO:2); AF153272 (SEQ ID NO:3); AW244797 (SEQ ID NO:4); XM_{—}001279 (SEQ ID NO:5); G49234 (SEQ ID NO:6); BQ839610 (SEQ ID NO:7); BC038282 (SEQ ID NO:8); AY418986 (SEQ ID NO:9); AY418987 (SEQ ID NO:10); AY418988 (SEQ ID NO:11); NM_{—}008556 (SEQ ID NO:12); NM_{—}011063 (SEQ ID NO:13); AJ583557 (SEQ ID NO:14); BC010469 (SEQ ID NO:15); BC022554 (SEQ ID NO:16); NM_{—}003768 (SEQ ID NO: 17); and BC002426 (SEQ ID NO: 18).

Exemplary PEA15 polypeptides (referred to by their number from the National Center for Biotechnology Information's GenBank database) include the following: AAD56775 (SEQ ID NO:19); XP_{—}001279 (SEQ ID NO:20); SEQ ID NO:21; Q15121 (SEQ ID NO:22); Q62048 (SEQ ID NO:23); AAH38282 (SEQ ID NO:24); NP_{—}032582 (SEQ ID NO:25); NP_{—}035193 (SEQ ID NO:26); NP_{—}003759 (SEQ ID NO:27); AAH10469 (SEQ ID NO:28); AAH22554 (SEQ ID NO:29); and AAH02426 (SEQ ID NO:30).

B. Percent Similarity

Percent similarity may be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e. nucleotides or amino acids) that are similar, divided by the total number of symbols in the shorter of the two sequences. The preferred default parameters for the GAP program include (1) a unitary comparison matrix (containing a value of 1 for identities and 0 for non-identities) of nucleotides and the weighted comparison matrix of Gribskov et al., 1986, as described by Schwartz et al., 1979; (2) a penalty of 3.0 for each gap and an additional 0.01 penalty for each symbol and each gap; and (3) no penalty for end gaps.

II. Nucleic Acid Sequences

In certain embodiments, the invention concerns the use of a PEA15 tumor-suppressing polynucleotides and gene products, such as the PEA15 that includes a sequence which is essentially that of the known PEA15 gene, or the corresponding protein. The term “a sequence essentially as PEA15” means that the sequence substantially corresponds to a portion of the hPEA15 or mPEA15 polynucleotide, for example, and has relatively few bases or amino acids (whether DNA or protein) that are not identical to those of the respective PEA15 (or a biologically functional equivalent thereof, when referring to proteins). The term “biologically functional equivalent” is well understood in the art and is further defined in detail herein. Accordingly, sequences which have between about 70% and about 80%; or more preferably, between about 81% and about 90%; or even more preferably, between about 91% and about 99%; of amino acids which are identical or functionally equivalent to the amino acids of PEA15 will be sequences which are “essentially the same”.

PEA15 polynucleotides that have functionally equivalent codons are also covered by the invention. The term “functionally equivalent codon” is used herein to refer to codons that encode the same amino acid, such as the six codons for arginine or serine, and also refers to codons that encode biologically equivalent amino acids (Table 1).

TABLE 1
FUNCTIONALLY EQUIVALENT CODONS.

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic | Asp | D | GAC | GAU | | | | |
| Acid | | | | | | | | |
| Glutamic | Glu | E | GAA | GAG | | | | |
| Acid | | | | | | | | |
| Phenyl- | Phe | F | UUC | UUU | | | | |
| alanine | | | | | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCU | | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The present invention also encompasses the use of DNA segments that are complementary, or essentially complementary, to the sequences set forth in the specification. Nucleic acid sequences that are “complementary” are those that are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment in question under relatively stringent conditions such as those described herein.

A. Biologically Functional Equivalents

As mentioned above, modification and changes may be made in the structure of PEA15 and still obtain a molecule having like or otherwise desirable characteristics. For example, certain amino acids may be substituted for other amino acids in a protein structure without appreciable loss of interactive binding capacity with structures such as, for example, ERK. Since it is the interactive capacity and nature of a protein that may define that protein's biological functional activity, certain amino acid sequence substitutions can be made in a protein sequence (or, of course, its underlying DNA coding sequence) and nevertheless obtain a protein with like or even countervailing properties (e.g., antagonistic vs. agonistic). It is thus contemplated that various changes may be made in the sequence of the PEA15 proteins or peptides (or underlying DNA) without appreciable loss of their biological utility or activity.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged. This is the case in the present invention, where any changes in the binding of PEA1S to ERK, for example, or, if applicable, binding of PEA15 to E1A, or any change that would render the peptide incapable of suppressing tumors or, if applicable, oncogenic-mediated transformation, would result in a loss of utility of the resulting peptide for the present invention.

Amino acid substitutions, such as those that might be employed in modifying PEA15 are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within +1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid.

B. Sequence Modification Techniques

Modifications to the PEA15 peptides may be carried out using techniques such as site-directed mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art as exemplified by publications (Adelman et al., 1983). As will be appreciated, the technique typically employs a phage vector that exists in both a single stranded and double stranded form. Typical vectors useful in site-directed mutagenesis include vectors such as the M13 phage (Messing et al., 1981). These phage are readily commercially available and their use is generally well known to those skilled in the art. Double stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart the two strands of a double stranded vector that includes within its sequence a DNA sequence that encodes the PEA15 gene. An oligonucleotide primer bearing the desired mutated sequence is prepared, generally synthetically, for example by the method of Crea et al. (1978). This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

Kalderon et al. (1984) report several mutagenic methods that have proved useful in mutating the native LT gene. Specifically, Kalderon et al. teach deletion mutations by displacement-loop mutagenesis and by the random insertion of EcoRI linkers into the LT gene. Further, point mutation by deletion-loop mutagenesis is taught. The reference also teaches screening procedures for determining the success of such mutations. The teachings of Kalderon et al. (1984) are incorporated by reference in this application.

The preparation of sequence variants of the selected gene using site-directed mutagenesis is provided as a means of producing potentially useful PEA15, or other neu-suppressing species and is not meant to be limiting as there are other ways in which sequence variants of these peptides may be obtained. For example, recombinant vectors encoding the desired genes may be treated with mutagenic agents to obtain sequence variants (see, e.g., a method described by Eichenlaub, 1979) for the mutagenesis of plasmid DNA using. hydroxylamine.

C. Other Structural Equivalents

In addition to the PEA15 peptidyl compounds described herein, it is contemplated that other sterically similar compounds may be formulated to mimic the key portions of the peptide structure. Such compounds may be used in the same manner as the peptides of the invention and hence are also functional equivalents. The generation of a structural functional equivalent may be achieved by the techniques of modeling and chemical design known to those of skill in the art. It will be understood that all such sterically similar constructs fall within the scope of the present invention.

III. Expression Vectors

In certain aspects of the present invention it may be necessary to express the PEA15 proteins. Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a PEA15 gene and translation of a PEA15 mRNA into a PEA15 protein product. In other embodiments, expression only includes transcription of the nucleic acid encoding a PEA 15 or its complement.

In order for the construct to effect expression of at least a PEA15 transcript, the polynucleotide encoding the PEA15 polynucleotide will be under the transcriptional control of a promoter. A “promoter” refers to a DNA sequence recognized by the synthetic machinery of the host cell, or introduced synthetic machinery, that is required to initiate the specific transcription of a gene. The phrase “under transcriptional control” means that the promoter is in the correct location in relation to the polynucleotide to control RNA polymerase initiation and expression of the polynucleotide. In some embodiments of the present invention, a heterologous promoter is utilized to regulate expression of a PEA15 polynucleotide. The term “heterologous promoter” as used herein refers to any regulatory region that is not the native regulatory region for PEA15. Heterologous promoters may be native regulatory regions for other polynucleotides, including those expressed tissue specifically, for example, or they could even be from other organisms, reporter sequence promoters, constitutive promoters, inducible promoters, and so forth.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter that is employed to control the expression of a PEA15 polynucleotide is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter. Where a human cancer cell is targeted, it is preferable provide a tissue-specific promoter, such as those exemplary tissue-specific promoters provided herein, others known in the art at the time of filing, and those to be identified after the filing of this application.

In various embodiments, the human cytomegalovirus (CMV) immediate early gene promoter, the SV40 early promoter and the Rous sarcoma virus long terminal repeat can be used to obtain high-level expression of the PEA15 polynucleotide. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosinase (melanoma), alpha-fetoprotein and albumin (liver tumors), CC10 (lung tumor) and prostate-specific antigen (prostate tumor) will permit tissue-specific expression of PEA15 polynucleotides. Table 2 lists several elements/promoters that may be employed, in the context of the present invention, to regulate the expression of PEA15 constructs. This list is not intended to be exhaustive of all the possible elements involved in the promotion of PEA 15 expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally, any promoter/enhancer combination (as per the Eukaryotic Promoter Data Base EPDB) could also be used to drive expression of a PEA15 construct. Use of a T3, T7 or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional genetic expression vector.

TABLE 2
TABLE 2 - Promoter and/or Enhancer

| Promoter/Enhancer | References |
|---|---|
| Immunoglobulin Heavy Chain | Banerji et al., 1983; Gilles et al., 1983; Grosschedl et |
| | al., 1985; Atchinson et al., 1986, 1987; Imler et al., |
| | 1987; Weinberger et al., 1984; Kiledjian et al., 1988; |
| | Porton et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et |
| | al., 1986; Kadesch et al., 1986; Wang et al., 1986; |
| | Ondek et al., 1987; Kuhl et al., 1987; Schaffner et al., |
| | 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and Villarreal., |
| | 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Chol et al., |
| | 1988; Reisman et al., 1989 |
| Papilloma Virus | Campo et al., 1983; Lusky et al., 1983; Spandidos and |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., |
| | 1987; Spandau et al., 1988; Vannice et al., 1988 |
| Human Immunodeficiency | Muesing et al., 1987; Hauber et al., 1988; Jakobovits |
| Virus | et al., 1988; Feng et al., 1988; Takebe et al., 1988; |
| | Rosen et al., 1988; Berkhout et al., 1989; Laspia et al., |
| | 1989; Sharp et al., 1989; Braddock et al., 1989 |
| Cytomegalovirus (CMV) | Weber et al., 1984; Boshart et al., 1985; Foecking et |
| | al., 1986 |
| Gibbon Ape Leukemia Virus | Holbrook et al., 1987; Quinn et al., 1989 |

Further, selection of a promoter that is regulated in response to specific physiologic signals can permit inducible expression of the PEA15 construct. For example, with the polynucleotide under the control of the human PAI-1 promoter, expression is inducible by tumor necrosis factor. Table 3 illustrates several exemplary promoter/inducer combinations:

TABLE 3
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa et |
| | | al., 1987, Karin et al., 1987; |
| | | Angel et al., 1987b; McNeall et |
| | | al., 1989 |
| MMTV (mouse | Glucocorticoids | Huang et al., 1981; Lee et al., |
| mammary tumor virus) | | 1981; Majors et al., 1983; |
| | | Chandler et al., 1983; Lee et |
| | | al., 1984; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H- | Interferon | Blanar et al., 1989 |
| 2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

In certain embodiments of the invention, the delivery of an expression vector in a cell may be identified in vitro or in vivo by including a marker in the expression vector. The marker would result in an identifiable change to the transfected cell permitting easy identification of expression. Usually the inclusion of a drug selection marker aids in cloning and in the selection of transformants. Alternatively, enzymes such as herpes simplex virus thymidine kinase (tk) (eukaryotic) or chloramphenicol acetyltransferase CAT) (prokaryotic) may be employed. Immunologic markers also can be employed. The selectable marker employed is not believed to be important, so long as it is capable of being expressed along with the polynucleotide encoding PEA15. Further examples of selectable markers are well known to one of skill in the art.

One typically will include a polyadenylation signal to effect proper polyadenylation of the transcript. The nature of the polyadenylation signal is not believed to be crucial to the successful practice of the invention, and any such sequence may be employed. The SV40 polyadenylation signal was employed in the exemplary studies in that it was convenient and known to function well in the target cells employed. Also contemplated as an element of the expression construct is a terminator. These elements can serve to enhance message levels and to minimize read through from the construct into other sequences.

The expression construct may comprise a virus or engineered construct derived from a viral genome. The ability of certain viruses to enter cells via receptor-mediated endocytosis and, in some cases, integrate into the host cell chromosomes, have made them attractive candidates for gene transfer in to mammalian cells. However, because it has been demonstrated that direct uptake of naked DNA, as well as receptor-mediated uptake of DNA complexes, expression vectors need not be viral but, instead, may be any plasmid, cosmid or phage construct that is capable of supporting expression of encoded genes in mammalian cells, such as pUC or Bluescript™ plasmid series.

IV. In Vivo Delivery and Treatment Protocols

Where the gene itself is employed to introduce the gene products, a convenient method of introduction will be through the use of a recombinant vector that incorporates the desired gene, together with its associated control sequences. The preparation of recombinant vectors is well known to those of skill in the art and described in many references, such as, for example, Sambrook et al. (1989), specifically incorporated herein by reference.

In vectors, it is understood that the DNA coding sequences to be expressed, in this case those encoding the PEA15 polynucleotide, are positioned adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter. One may also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of the PEA15 will be preferred, there is no reason why other control sequences could not be employed, so long as they are compatible with the genotype of the cell being treated. Thus, one may mention other useful promoters by way of example, including, e.g., an SV40 early promoter, a long terminal repeat promoter from retrovirus, an actin promoter, a heat shock promoter, a metallothionein promoter, and the like.

For introduction of the PEA15 gene, it is proposed that one will desire to preferably employ a vector construct that will deliver the desired gene to the affected cells. This will, of course, generally require that the construct be delivered to the targeted tumor cells, for example, breast, genital, or lung tumor cells. It is proposed that this may be achieved most preferably by introduction of the desired gene through the use of a viral or non-viral vector to carry the PEA15 sequences to efficiently transfect the tumor, or pretumorous tissue. This infection may be achieved preferably by liposomal delivery but may also be via adenoviral, a retroviral, a vaccinia viral vector or adeno-associated virus. These vectors have been successfully used to deliver desired sequences to cells and tend to have a high infection efficiency.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment that also contains the SV40 viral origin of replication. Smaller or larger SV40 fragments may also be used, provided there is included the approximately 250 bp sequence extending from the Hind III site toward the Bgl I site located in the viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

A. Liposomal Transfection

Thus the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

The present invention also provides particularly useful methods for introducing neu-suppressing gene products into cells. One method of in vivo gene transfer that can lead to expression of genes transfected into cells involves the use of liposomes. Liposomes can be used for both in vitro and in vivo transfection. Liposome-mediated gene transfer seems to have great potential for certain in vivo applications in animals (Nicolau et al., 1987). Studies have shown that intravenously injected liposomes are taken up essentially in the liver and the spleen, by the macrophages of the reticuloendothelial system. The specific cellular sites of uptake of injected liposomes appear to be mainly spleen macrophages and liver Kupffer cells. Intravenous injection of liposomes/DNA complexes can lead to the uptake of DNA by these cellular sites, and result in the expression of a gene product encoded in the DNA (Nicolau, 1982).

It is contemplated that oncogenic-suppressing, for example ras- or neu-suppressing, gene products can be introduced into cells using liposome-mediated gene transfer. It is proposed that such constructs can be coupled with liposomes and directly introduced via a catheter, as described by Nabel et al. (1990). By employing these methods, the PEA15 tumor suppressing gene product can be expressed efficiently at a specific site in vivo, not just the liver and spleen cells that are accessible via intravenous injection. Therefore, this invention also encompasses compositions of DNA constructs encoding a PEA15 tumor suppressor gene product formulated as a DNA/liposome complex and methods of using such constructs.

Liposomal transfection can be via liposomes composed of, for example, phosphatidylcholine (PC), phosphatidylserine (PS), cholesterol (Chol), N-[1-(2,3-dioleyloxy)propyl]-N,N-trimethylammonium chloride (DOTMA), dioleoylphosphatidylethanolamine (DOPE), and/or 3.beta.[N—(N′N′-dimethylaminoethane)-carbarmoyl cholesterol (DC-Chol), as well as other lipids known to those of skill in the art. Those of skill in the art will recognize that there are a variety of liposomal transfection techniques which will be useful in the present invention. Among these techniques are those described in Nicolau et al. (1987); Nabel et al. (1990); and Gao et al. (1991). Particular success was found with liposomes comprising DC-Chol. More particularly, success was found with liposomes comprising DC-Chol and DOPE that have been prepared following the teaching of Gao et al., 1991. It is also anticipated that liposomes comprised of DOTMA, such as those that are available commercially under the trademark Lipofectin™, from Vical, Inc., in San Diego, Calif. will be useful. In a preferred embodiment, however, a PEA composition is comprised in a LPD liposome, wherein the LPD liposome comprises a cationic liposome/DNA complex comprised of a DOTAP:cholesterol liposome, protamine sulfate, and plasmid DNA.

Liposomes may be introduced into contact with cells to be transfected by a variety of methods. In cell culture, the liposome-DNA complex can simply be dispersed in the cell culture solution. For application in vivo, liposome-DNA complex are typically injected. Intravenous injection allows liposome-mediated transfer of DNA complex, for example, the liver and the spleen. In order to allow transfection of DNA into cells that are not accessible through intravenous injection, it is possible to directly inject the liposome-DNA complexes into a specific location in an animal's body. For example, Nabel et al. teach injection via a catheter into the arterial wall. In another example, intraperitoneal injection was used to allow for gene transfer into mice.

The present invention also contemplates compositions comprising a liposomal complex. This liposomal complex will comprise a lipid component and a DNA segment encoding a tumor suppressor polynucleotide. The tumor suppressor polynucleotide employed in the liposomal complex can be, for example, a PEA15 gene.

The lipid employed to make the liposomal complex can be any of the above-discussed lipids. In particular, DOTMA, DOPE, DC-Chol, and/or LPD may form all or part of the liposomal complex. In a preferred embodiment, the lipid will comprise LPD. Regarding embodiments wherein DC-Chol and DOPE are utilized, while any ratio of DC-Chol to DOPE is anticipated to have utility, it is anticipated that those comprising a ratio of DC-Chol:DOPE between about 1:20 and 20:1 will be particularly advantageous. It known that liposomes prepared from a ratio of DC-Chol:DOPE of about 1:10 to about 1:5 have been useful.

It is proposed that it will ultimately be preferable to employ the smallest region of PEA15 comprising tumor suppressor activity so that one is not introducing unnecessary DNA into cells which receive a PEA15 gene construct. Techniques well known to those of skill in the art, such as the use of restriction enzymes, will allow for the generation of small regions of PEA15. The ability of these regions to act as a tumor suppressor can easily be determined by the assays reported herein.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

B. Adenovirus

Another method for in vivo delivery involves the use of an adenovirus vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

Adenovirus is a particularly suitable gene transfer vector because of its midsized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, located at 16.8 m.mu. is particularly efficient during the late phase of infection, and all the mRNA's issued from this promoter possess a 5′-tripartite leader (TL) sequence which makes them preferred mRNA's for translation.

In some cases, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure. Use of the YAC system is an alternative approach for the production of recombinant adenovirus.

A particular method of introducing the PEA15 to an animal is to introduce a replication-deficient adenovirus comprising the PEA15 gene or a fragment thereof encoding a gene product comprising tumor suppressor activity. The replication-deficient construct made by E1B and E3 deletion also avoids the viral reproduction inside the cell and transfer to other cells and infection of other people, which means the viral infection activity is shut down after it infects the target cell. The PEA15 gene is still expressed inside the cells. Also, unlike retrovirus, which can only infect proliferating cells, adenovirus is able to transfer the PEA15 gene into both proliferating and non-proliferating cells. Further, the extrachromosomal location of adenovirus in the infected cells decreases the chance of cellular oncogene activation within the treated animal.

The nature of the adenovirus vector is not believed to be crucial to the successful practice of the invention. Of course, as discussed above, it is advantageous if the adenovirus vector is replication defective, or at least conditionally defective. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers, e.g., 109-1011 plaque-forming units per ml, and they are highly infective. The life cycle of adenovirus does not require integration in to the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenoviruses have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotatic inoculation into the brain (Le Gal La Salle et al., 1993).

Introduction of the adenovirus containing PEA15 polynucleotide into a suitable host is typically done by injecting the virus contained in a buffer.

C. Retroviruses

The retroviruses are a group of single-stranded RNA viruses characterized by an ability to convert their RNA to double-stranded DNA to infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene, termed .PSI. components is constructed (Mann et al., 1983). When a recombinant plasmid containing a human cDNA, together with the retroviral LTR and PSI sequences is introduced into this cell line (by calcium phosphate precipitation for example), the PSI sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact PSI sequence from the recombinant virus inserts upstream from the gag, pol, env sequence integrated in the host cell genome. However, neu packaging cell lines are now available that should greatly decrease the likelihood of recombination (Markowitz et al., 1988; Hersdorffer et al., 1990).

One limitation to the use of retrovirus vectors in vivo is the limited ability to produce retroviral vector titers greater than 10⁶ infections U/mL. Titers 10- to 1.000-fold higher are necessary for many in vivo applications.

Several properties of the retrovirus have limited its use in lung cancer treatment (Stratford-Perricaudet and Perricaudet, 1991; (i) Infection by retrovirus depends on host cell division. In human cancer, very few mitotic cells can be found in tumor lesions. (ii) The integration of retrovirus into the host genome may cause adverse effects on target cells, because malignant cells are high in genetic instability. (iii) Retrovirus infection is often limited by a certain host range. (iv) Retrovirus has been associated with many malignancies in both mammals and vertebrates. (v) The titer of retrovirus, in general, is 100- to 1.000-fold lower than that of adenovirus.

D. Other Viral Vectors as Expression Constructs

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpes viruses may be employed. They offer several attractive features for various mammalian cells (Friedmann, 1989; Ridgeway, 1988; Baichwal and Sugden, 1986; Coupar et al, 1988; Howrich et al., 1990).

With the recognition of defective hepatitis B viruses, neu insight was gained into the structure-function relationship of different viral sequences. In vitro studies showed that the virus could retain the ability for helper-dependent packaging and reverse transcription despite the deletion of up to 80% of its genome (Horwich et al, 1990). This suggested that large portions of the genome could be replaced with foreign genetic material. The hepatotropism and persistence (integration) were particularly attractive properties for liver-directed gene transfer. Chang et al. introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Cultures media containing high titers of the recombinant virus were used to infect primary duckling hepatocytes. Stable CAT gene expression was detected for at least 24 days after transfection (Chang et al., 1991).

E. Other Non-Viral Vectors

In order to effect expression of sense or antisense gene constructs, the expression construct must be delivered into a cell. This delivery may be accomplished in vitro, as in laboratory procedures for transforming cells lines, or in vivo or ex vivo (see below), as in the treatment of certain disease states. As described above, delivery may be via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

Other expression constructs that can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells, in vitro, and then the return of the modified cells back into an animal. This may involve the surgical removal of tissue/organs from an animal or the primary culture of cells and tissues. Anderson et al., U.S. Pat. No. 5,399,346, and incorporated herein in its entirety, disclose ex vivo therapeutic methods.

V. Combined Therapy Protocols

Tumor cell resistance to DNA damaging agents represents a major problem in clinical oncology. The present invention may also be used in combination with conventional therapies to improve the efficacy of chemotherapy, radiotherapy, gene therapy, immunotherapy, hormonal therapy, and surgery, for example. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that PEA15 therapy could be used similarly in conjunction with chemo- or radiotherapeutic intervention.

To kill cells, such as malignant or metastatic cells, using the methods and compositions of the present invention, one may generally contact a “target” cell with a PEA15 composition and at least one DNA damaging agent. These compositions would be provided in a combined amount effective to kill or inhibit proliferation of the cell. This process may involve contacting the cells with the PEA15 composition and the DNA damaging agent(s) or factor(s) at the same time. This may be achieved by contacting the cell with a single composition or pharmacological formulation that includes both agents, or by contacting the cell with two distinct compositions or formulations, at the same time, wherein one composition includes the PEA15 composition and the other includes the DNA damaging agent.

Alternatively, the PEA15 treatment may precede or follow the DNA damaging agent treatment by intervals ranging from minutes to weeks. In embodiments where the DNA damaging factor and PEA15 are applied separately to the cell, one would generally ensure that a significant period of time did not expire between the time of each delivery, such that the DNA damaging agent and PEA15 composition would still be able to exert an advantageously combined effect on the cell. In such instances, it is contemplated that one would contact the cell with both agents within about 6 hours to one week of each other and, more preferably, within about 24-72 hours of each other, with a delay time of only about 48 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the PEA15 or the DNA damaging agent will be desired. Various combinations may be employed, where PEA15 is “A” and the DNA damaging agent is “B”:

| | | | | | |
|---|---|---|---|---|---|
| A/B/A | B/A/B | B/B/A | A/A/B | B/B/B/A | B/B/A/B |

| | | | | | |
|---|---|---|---|---|---|
| A/A/B/B | A/B/A/B | A/B/B/A | B/B/A/A | B/A/B/A | B/A/A/B |
| A/A/A/B | B/A/A/A | A/B/A/A | A/A/B/A | A/B/B/B | B/A/B/B |

To achieve cell killing, both agents are delivered to a cell in a combined amount effective to kill the cell.

DNA damaging agents or factors are defined herein as any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents”, function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide.

In treating cancer according to the invention, one would contact the tumor cells with a DNA damaging agent in addition to the PEA15 composition. This may be achieved by irradiating the localized tumor site with DNA damaging radiation such as X-rays, UV-light, γ-rays or even microwaves. Alternatively, the tumor cells may be contacted with the DNA damaging agent by administering to the subject a therapeutically effective amount of a pharmaceutical composition comprising a DNA damaging compound such as, adriamycin, 5-fluorouracil, etoposide, camptothecin, actinomycin-D, mitomycin C, or more preferably, cisplatin. The DNA damaging agent may be prepared and used as a combined therapeutic composition, or kit, by combining it with a PEA 15 composition, as described above.

Agents that directly cross-link polynucleotides, specifically DNA, are envisaged and are shown herein, to eventuate DNA damage leading to a synergistic antineoplastic combination. Agents such as cisplatin, and other DNA alkylating may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors and subunits also lead to DNA damage. As such a number of polynucleotide precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as y-rays, X-rays, and/or the directed delivery of radioisotopes to tumor cells. Other forms of DNA damaging factors are also contemplated such as microwaves and UV-irradiation. It is most likely that all of these factors effect a broad range of DNA damage, or the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It is proposed herein that the regional delivery of PEA15 compositions to patients with tumors will be a very efficient method for delivering a therapeutically effective gene to counteract the clinical disease. Similarly, the chemo- or radiotherapy may be directed to a particular, affected region of the subject's body. Alternatively, systemic delivery of the PEA15 or the DNA damaging agent may be appropriate in certain circumstances, for example, where extensive metastasis has occurred.

Cytokine therapy also has proven to be an effective partner for combined therapeutic regimens. Various cytokines may be employed in such combined approaches. Examples of cytokines include IL-1α, IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNFα, TNFβ, LAF, TCGF, BCGF, TRF, BAF, BDG, MP, LIF, OSM, TMF, PDGF, IFN-α, IFN-β, IFN-γ. Cytokines are administered according to standard regimens, as described below, consistent with clinical indications such as the condition of the patient and relative toxicity of the cytokine.

The present invention also contemplates the use of a combination of therapies in the treatment of cancers, particularly those comprising, for example, E1A tumor suppressor activity, those cancers where administration of PEA15 facilitates E1A tumor suppressor activity, and/or those cancers where treatment with PEA15 promotes cytoplasmic localization of ERK. In particular, studies herein suggest that PEA15 gene product results in the chemosensitization of cancer cells to chemotherapeutic agents, such as the exemplary paclitaxel. The present invention contemplates using PEA15 constructs in combination with chemotherapeutic agents such as paclitaxel to affect the treatment of cancers affectable thereby.

VI. Pharmaceutical Compositions and Routes of Administration

Compositions of the present invention will have an effective amount of a polynucleotide for therapeutic administration in combination with an effective amount of a compound (second agent) that is a cancer-treating agent, such as a chemotherapeutic agent as exemplified above. Such compositions, such as a therapeutically effective amount, will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by regional, local, intratumoral, pre- and/or post-surgical, orthotopic, intradermal, subcutaneous, intramuscular, intraperitoneal or intravenous injection. Such compositions would normally be administered as pharmaceutically acceptable compositions, described supra. In embodiments wherein the breast or ovary is being treated, administration of the pharmaceutical composition may comprise injection into a surrounding or nearby cavity. Alternatively, or in addition to, the pharmaceutical composition may be administered systemically.

The vectors of the present invention and, where applicable, the liposomal complexes, are advantageously administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a mouthwash, cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All the essential materials and reagents required for inhibiting tumor cell proliferation may be assembled together in a kit. When the components of the kit are provided in one or more liquid solutions, the liquid solution preferably is an aqueous solution, with a sterile aqueous solution being particularly preferred.

For in vivo use, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

### Parenteral Administration

The active compounds of the present invention will often be formulated for parenteral administration, e.g., formulated for injection via the intravenous, intramuscular, sub-cutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds, as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of cancerous tissues may be accomplished in any one of a variety of ways. Plasmid vectors and retroviral vectors, adenovirus vectors, and other viral vectors all present means by which to target human cancers. It is anticipated that particular success for the use of liposomes to target PEA15 genes to cancer cells will occur. In one of the first series of clinical phases to be performed, DNA encoding PEA15 will be complexed with liposomes in the manner described above, and this DNA/liposome complex will be injected into patients with certain forms of cancer, such as breast cancer, intravenous injection can be used to direct the gene to all cells, including those which are transformed or cancerous. Directly injecting the liposome complex into the proximity of a cancer can also provide for targeting of the complex with some forms of cancer. For example, cancers of the ovary can be targeted by injecting the liposome mixture directly into the peritoneal cavity of patients with ovarian cancer. Of course, the potential for liposomes that are selectively taken up by a population of cancerous cells exists, and such liposomes will also be useful for targeting the gene.

Those of skill in the art will recognize that the best treatment regimens for using PEA15 to suppress cancers such as in tumors can be straightforwardly determined. This is not a question of experimentation, but rather one of optimization, which is routinely conducted in the medical arts. The in vivo studies in nude mice provide a starting point from which to begin to optimize the dosage and delivery regimes. The frequency of injection will initially be once a week, as was done in the mice studies. However, this frequency might be optimally adjusted from one day to every two weeks to monthly, depending upon the results obtained from the initial clinical trials and the needs of a particular patient. Human dosage amounts can initially be determined by extrapolating from the amount of PEA15 used in mice, approximately 15 μg of PEA15 DNA per 50 g body weight. Based on this, a 50 kg woman would require treatment with 15 mg of DNA per dose. In certain embodiments it is envisioned that this dosage may vary from between about 100 μg/50 g body weight to about 5 μg/g body weight; or from about 90 μg/50 g body weight to about 101g/g body weight or from about 80 μg/50 g body weight to about 15 μg/g body weight; or from about 75 μg/50 g body weight to about 20 μg/g body weight; or from about 60 μg/50 g body weight to about 30 μg/g body weight about 50 μg/50 g body weight to about 40 μg/g body weight. In other embodiments this dose may be about 5, 8, 10 15, or 20 μg/50 g. Of course, this dosage amount may be adjusted upward or downward, as is routinely done in such treatment protocols, depending on the results of the initial clinical trials and the needs of a particular patient.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### E1A Reduced Proliferation and Induced Apoptosis in Cancer Cells

This present example demonstrates with the exemplary E1A gene the manipulation of cancer cells to demonstrate cell growth modulation, such as of reduced cell proliferation and induction of apoptosis.

Transfection of E1A into Cancer Cells by Using a Cationic Liposome

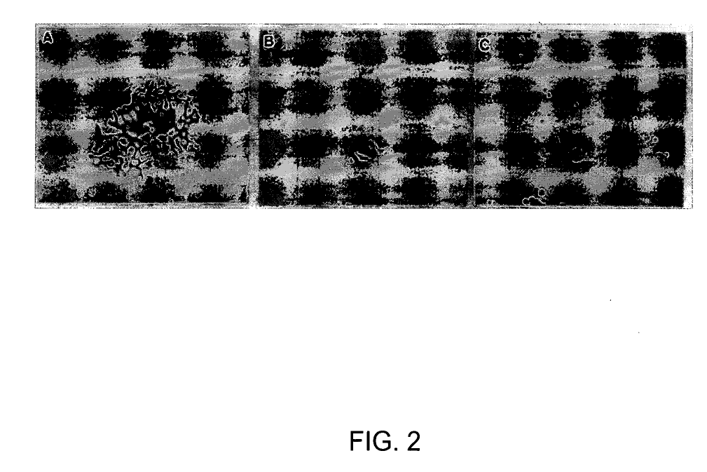

The safety and the efficiency of gene transfer with the DCC-E1A cationic liposome complex was characterized. Eighteen patients were given weekly injections of the DCC-E1A complex through a Tenckhoff catheter placed in either the pleural cavity (for patients with breast cancer) or the peritoneal cavity (for patients with ovarian cancer). The cycles lasted for 4 weeks; each cycle consisted of 3 weekly injections of the DCC-E1A complex followed by 1 week of no injections. During the course of the phase I trial, the E1A gene dose was doubled twice, from 1.8 to 3.6 and then to 7.2 mg/m² per injection. The median number of injections given to patients was 6 (range, 1-8). To confirm the preclinical findings (Yu et al., 1995; Chang et al., 1997), it was first examined whether HER2 expression levels were downregulated in HER2-overexpressing cancer cells after administration of DCC-E1A. After completion of the injection schedule, adjoining sections of the tumor cells were immunohistochemically stained for HER2 and E1A and analyzed for the distribution of E1A gene expression. HER2 downregulation was indeed seen in tumor cells that expressed E1A (FIG. 2). Similar findings were observed in other patients.

### E1A Enhanced Apoptosis and Reduced Proliferation of Cancer Cells

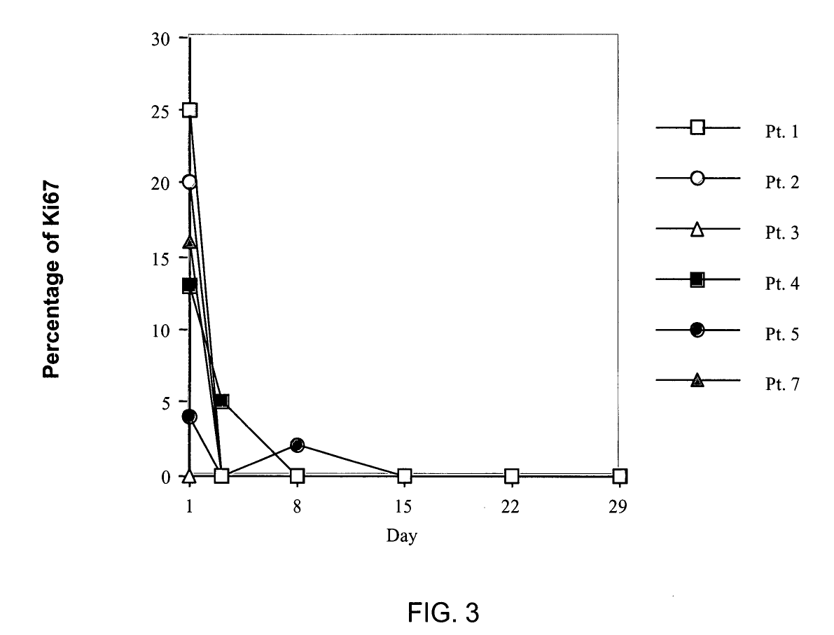

After it was confirmed that HER2 expression had been downregulated after administration of the DCC-E1A complex, the present inventors looked for evidence of decreases in tumor in the intracavitary fluid over the course of the DCC-E1A complex administration. When ascites or pleural fluids collected from 6 patients before and after treatments were compared, the percentage of tumor clumps decreased dramatically after administration of the DCC-E1A complex. Because E1A itself is known to induce apoptosis and may contribute to reductions in tumor cell number, we examined tumor-cell apoptosis with TUNEL assays. The percentage of apoptotic cells increased after administration of the DCC-E1A complex. The present inventors also looked for decreases in DNA replication (proliferation) in these tumor cells by examining Ki-67 expression. Ki-67 expression dropped within 15 days after the first delivery of DCC-E1A complex (FIG. 3). Interestingly, this increase in apoptosis and decrease in Ki-67 expression in E1A-transfected cells did not correlate with extent of HER2 downregulation or baseline HER2 level in the cancer cells. In a specific embodiment of the present invention, this indicates that E1A may reduce cell proliferation (DNA synthesis) and induce apoptosis by some mechanism independent of HER2.

### Example 2

### E1A Suppressed Proliferation and Tumorigenicity of Cancer Cells

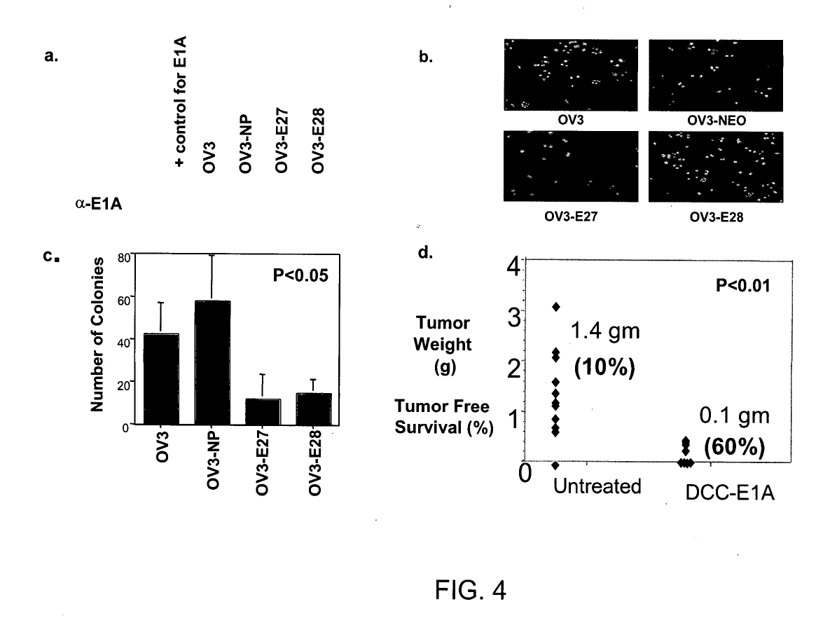

As noted elsewhere herein, the precise mechanism by which E1A acts as a tumor suppressor in low-HER2 expressing cells was unclear. To address this issue, the present inventors transfected E1A into the exemplary low-HER2-expressing human ovarian cancer cell line OVCAR3 (OV3) to constitutively express E1A (OV3-E1A) and compared the biological characteristics of the transfectants with those of the parental OV3 cell line (FIG. 4A). The DNA synthesis rate (measured by BrdU incorporation) (FIG. 4B) and anchorage-independent growth of the OV3-E1A cells were both suppressed by 64% relative to the parental cells (P<0.0001) (FIG. 4C). Further experiments with nude mice bearing OV3 cells in the peritoneal cavity confirmed that weekly injections of E1A complexed with DC-Chol cationic liposome (DCC-E1A) resulted in smaller tumors and extended the disease-free survival period (FIG. 4D).

### Example 3

### E1A-Induced PEA15 Sequesters ERK from Nucleus to Cytoplasm in Cancer Cells

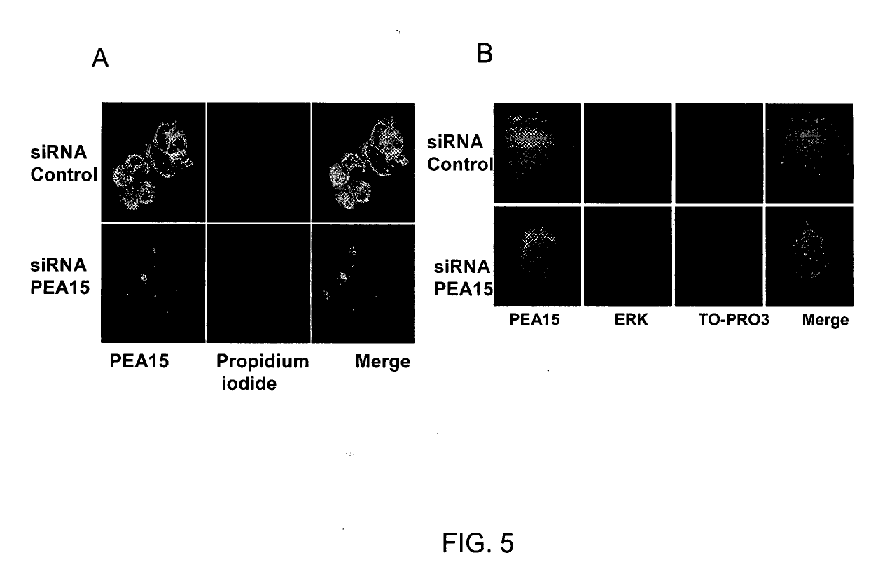

To further examine reduction of DNA synthesis in OV3 cell contributing to less tumorigenicity, the present investigators further characterized the role of PEA15 in OV3-E1A cells. The present inventors identified PEA15 as a potential target gene of E1A by comparing the gene expression profiles of OV3 and OV3-E1A cells and confirmed that PEA15 levels were much higher in the cytoplasm of OV3-E1A cells than in the cytoplasm of OV3 cells. Because PEA15 blocks ERK-dependent proliferation by binding to activated (phosphorylated) pERK in the cytoplasm and preventing the translocation of that ERK to the nucleus (Formstecher et al., 2001), it was tested whether ERK localization in cytoplasm depended on PEA 15 in E1A-transfected cancer cells. When PEA15 was “knocked down” by siRNA, ERK accumulated in the nucleus instead of the cytoplasm (FIG. 5). These results strongly suggest that sequestration of pERK (active form) in the cytoplasm may block ERK phosphorylation of nuclear substrates, which in a specific embodiment of the present invention is important for suppression of tumorigenicity.

### Example 4

### PEA15 Reduced Colony Formation and Anchorage-Independent Growth of Cancer Cells

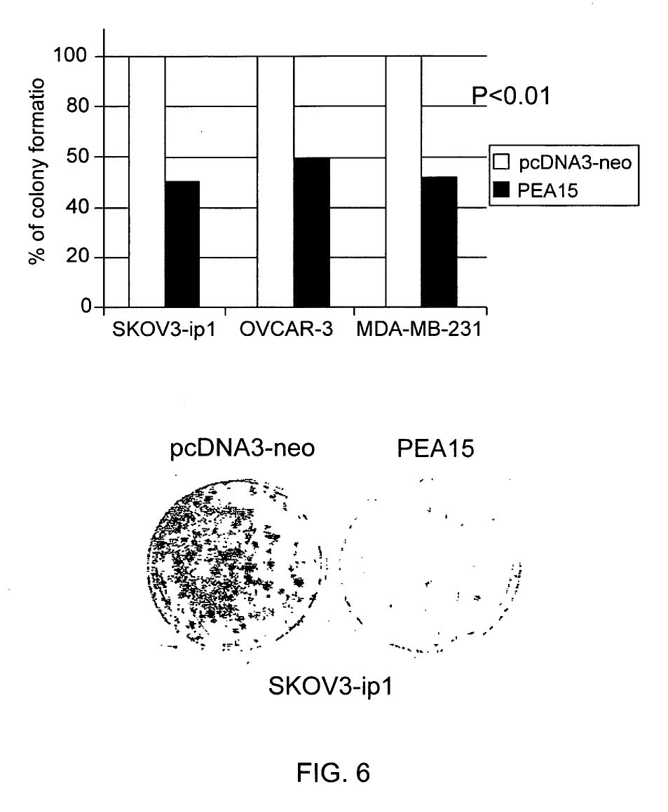

Because PEA15 contributes to reduce cell proliferation in OV3 cells, in a specific embodiment, the present inventors explored whether PEA15 acts as a tumor suppression gene in human breast and ovarian cancer, as only exemplary forms of cancer. Colony formation was examined after transfection of two marker genes into human ovarian and breast cancer cell lines (FIG. 6). One exemplary construct, pcDNA-PEA15-neo, encodes the PEA15 gene coupled with the neomycin resistance gene; the other construct, pSV2-neo, encodes only the neomycin resistance gene. Breast cancer (MDA-MB-435) and ovarian cancer (SKOV3-ip1) cell lines that had been transfected with pcDNA-pPEA15-neo formed fewer colonies than did the same cell lines that had been transfected with pcDNA-neo (P<0.01), which indicates there is a tumor-suppressive effect.

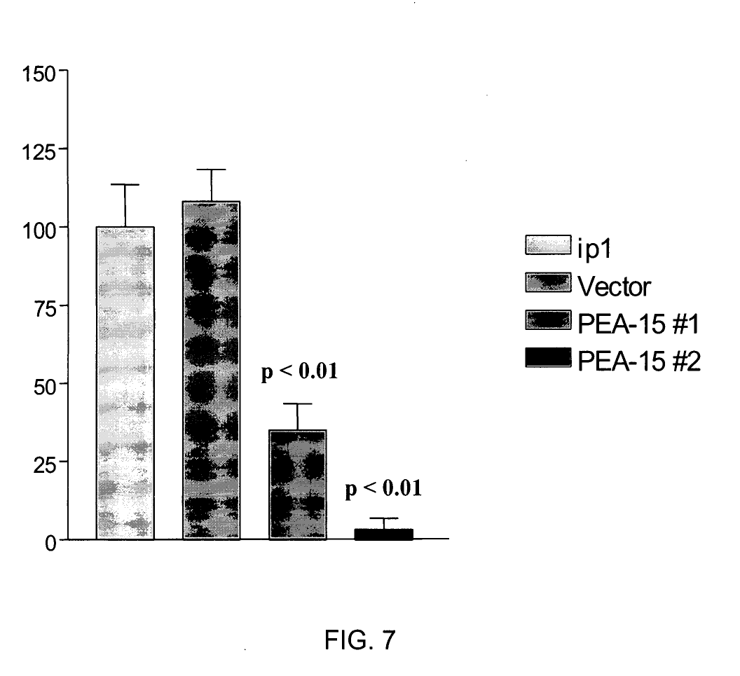

To further characterize this colony suppression, the present inventors established transfectants that constitutively express PEA 15 by screening for PEA 15 expression among SKOV3-ip1 colonies that had been transfected with pcDNA-pPEA15-neo. Two PEA15 transfectant clones (ip1-PEA15 #1, ip1-PEA15 #2) were identified and examined in comparison with the parental cell line and the vector-control transfectant (ip1-NP, vector) (FIG. 7). Again, the PEA15 stable transfectant produced significantly fewer anchorage-independent colonies than the control cells.

### Example 5

### PEA15 Enhanced Paclitaxel-Induced Cell Death

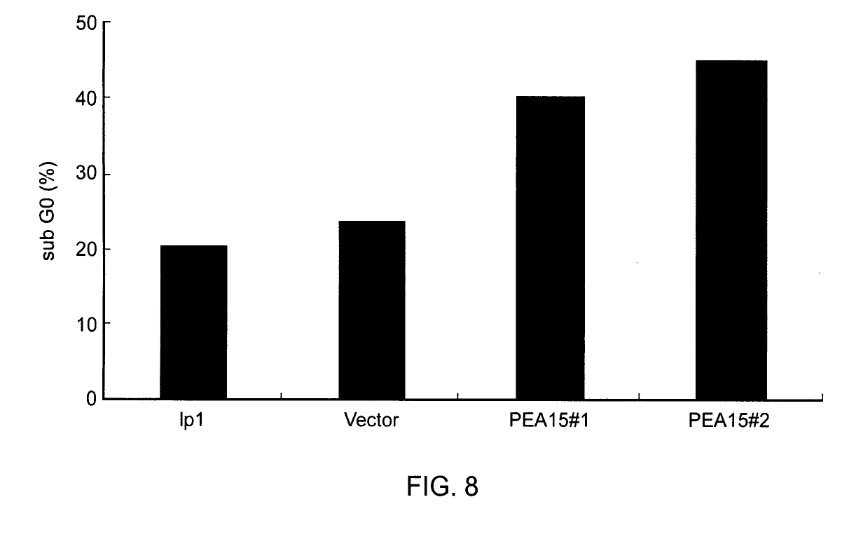

To characterize whether PEA15 sensitizes human ovarian cancer cell lines to paclitaxel, two stably transfected ip1 ovarian cancer cell lines-ip1-PEA15 #1, and ip1-PEA15 #2-were established. By pooling SKOV3-ip1 cells transfected with the neomycin resistance gene, the cell line SKOV3-ip1-NP (vector alone) was established as a positive control. PEA15 overexpression correlated with enhanced paclitaxel-induced apoptosis, as shown in FIG. 8. Chemosensitivity was monitored by MTT assay 72 h following paclitaxel exposure. Parental SKOV3-ip1 cells were more resistant to paclitaxel (0.1 μM) than were PEA15-overexpressing SKOV3-ip1 cells.

### Example 6

### Systemic Gene Delivery with the Novel LPD Cationic Liposome

Gene therapy has largely been limited to local gene delivery. In specific embodiments of the present invention, therapeutic polynucleotides are delivered systemically, which should be more effective, and possibly more convenient, for the treatment of metastatic disease. It was tested whether the novel cationic liposome LPD could be used as a systemic gene delivery system for E1A gene therapy in breast cancer.

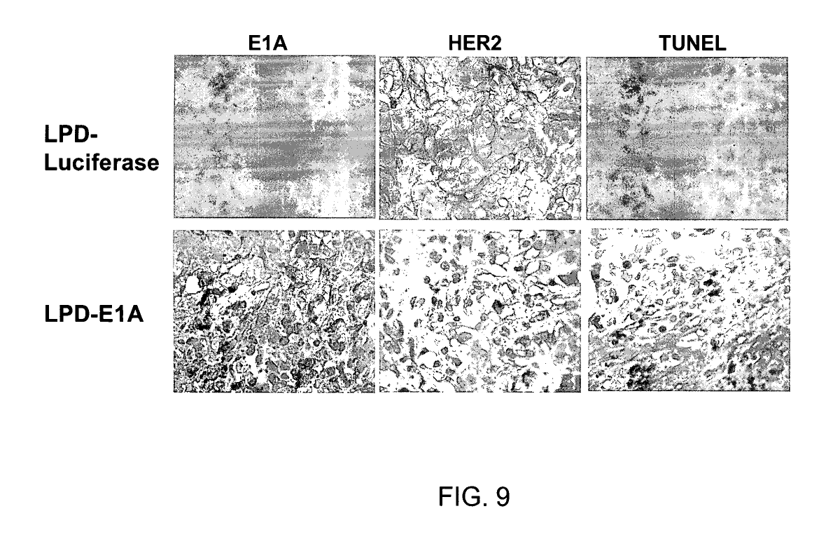

Expression and function of E1A protein after systemic administration of LPD-E1A is demonstrated in FIG. 8. It was first tested whether E1A could be delivered with the LPD formulation. Immunohistochemical analysis was performed on tumor tissue sections obtained from mice bearing MDA-MB-361 tumors 48 hours after treatment. Animals given intravenous LPD-E1A (20 μg DNA) demonstrated expression of E1A protein within the peritumoral areas, but the control treated animals did not express E1A (FIG. 9) (Yu et al., 1990). Coinciding with the expression of E1A was a downregulation of HER2 protein expression within the peritumoral area in the LPD-E1A-treated samples, whereas intense staining was still present in the control-treated samples (FIG. 9, bottom panels). Sections from the LPD-E1A-treated animals also had TUNEL-positive apoptotic tumor cells in the E1A-transfected area (FIG. 9, bottom panels). Again, in tumor tissue sections obtained from control mice treated with LPD-luciferase (control DNA), no E1A expression, no downregulation of HER2 expression, and no apoptosis induction were observed (FIG. 9, top panels). These results clearly demonstrate that functional E1A can be expressed at distant tumor sites after systemic administration of LPD.

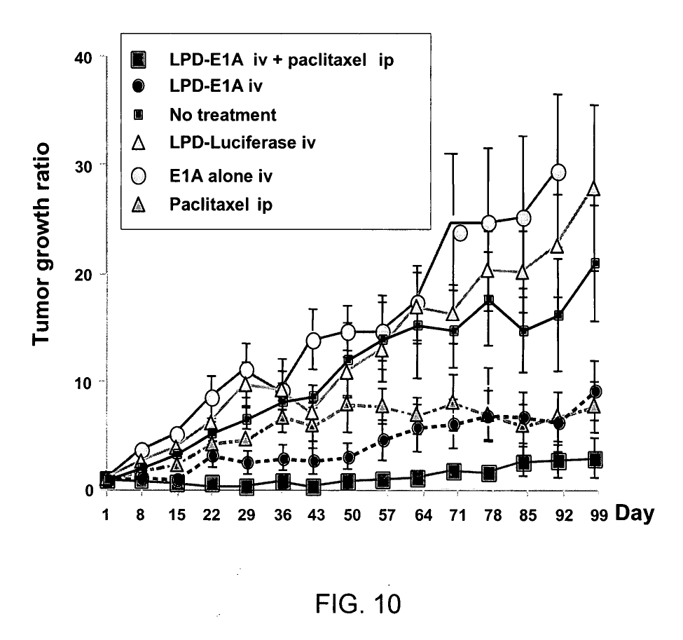

LPD-E1A enhanced the in vivo chemosensitivity of breast cancer xenografts to paclitaxel, as shown in FIG. 10. Previous preclinical experiments have shown that HER2-overexpressing breast cancer cell lines are resistant to paclitaxel (Ueno et al., 1997; Yu et al., 1996; Yu et al., 1998; Yu et al., 1998). On the basis of these in vitro findings, the present inventors examined systemic E1A gene therapy and its enhancement of the efficacy of paclitaxel in an exemplary MDA-MB-361 breast cancer xenograft model. Treatment with 20 μg of naked E1A DNA, LPD alone, or LPD-Luciferase (20 μg DNA) did not suppress tumor growth. However, treatment with either LPD-E1A (20 μg DNA) (P=0.0253) or paclitaxel (15 mg/kg, i.p.) (P=0.0644) decreased tumor growth as compared with LPD-Luciferase (20 μg DNA). Moreover, the combination of LPD-E1A and paclitaxel elicited the greatest anti-tumor effect as compared with LPD-Luciferase (P=0.0071), paclitaxel (P=0.0104), or LPD-E1A (P=0.0490). Finally, significantly fewer tumors were present in the animals treated with both paclitaxel and LPD-E1A as compared with the other treatment groups (P=0.0106). The combination of LPD-E1A and paclitaxel clearly enhanced the suppression of tumor growth over that of either agent alone, demonstrating robust additive effects between the two agents (Ueno et al., 2002). These findings indicate that PEA15 can be delivered by using the LPD system. In keeping with routine modifications of such a system in the art, the present inventors will identify the most efficient schedule (e.g., daily, weekly, or other) for delivering the gene of interest. It is also determined whether the LPD system can be used to deliver genes to tumors located in the peritoneal cavity, utilizing analogous methods that may require some modifications known by those of skill in the art.

### Example 7

### Anti-Tumor Effect of E1A in Breast and Ovarian Cancer Cells Depends ON PEA15

In a specific embodiment of the present invention, E1A suppresses tumorigenicity by upregulating PEA15. PEA15 has been shown to block ERK-dependent proliferation by binding ERK and preventing ERK from being translocated into the nucleus. The cytoplasmic sequestration of ERK results in blocking the ability of ERK to phosphorylate the transcription factor Elk-1, which promotes ERK-dependent transcription (Formstecher et al., 2001; Gille et al., 1995). As indicated elsewhere herein, in a specific embodiment upregulation of PEA15 by E1A is a mechanism underlying the anti-tumor activity of E1A.

In a further specific embodiment of the present invention, E1A suppresses tumorigenicity in tumor cells by upregulating PEA15, which sequesters ERK into the cytoplasm and slows proliferation of tumor cells. As described below, the upregulation of PEA15 is confirmed by infecting several exemplary breast and ovarian cancer cell lines with E1A cDNA by using an exemplary adenoviral vector, Ad.E1A(+). It is also determined whether PEA15 sequesters ERK in the cytoplasm. Further, it is determined whether PEA15 is required for anti-tumor effect of E 1A.

Determination of E1A Upregulation of PEA15 in Cancer Cells Transiently Transfected with E1A

PEA15 is upregulated in human ovarian cancer cells that constitutively express E1A. The generality of PEA 15 upregulation by E1A in other breast cancer and ovarian cancer cell lines is determined. This study addresses the issue of clonal selection in stable transfectants by using transient transfection. E1A is delivered by means of an adenoviral vector. The first steps are to titrate the multiplicity of infection (MOI) of the adenoviral vector with cDNA of β-galactosidase, Ad. β-gal(+) to identify the optimal conditions for achieving the greatest transfection efficiency with the least toxicity. A variety of ovarian cancer cell lines [e.g., OVCAR3, SKOV3-ip1, 2774] and breast cancer cell lines [e.g., SK-Br-3, MDA-MB-453, MDA-MB-435, MDA-MB-231] that vary in their constitutive expression of HER2 are transfected. Protein samples are collected after the cells have been treated with E1A (delivered by adenovirus), and then HER2, PEA15 and E1A protein expression is analyzed by western blotting and immunohistochemical staining with a monoclonal anti-PEA15 antibody (provided by collaborator Dr. Ginsberg) as well as anti-HER2 and anti-E1A antibodies. Western blotting allows one to assess the overall E1A and PEA15 expression in a population of cells, and immunohistochemical staining determines the number of cells staining positive for E1A and PEA15. The finding of HER2 downregulation by E1A in HER2-overexpressing cells serves as confirmation that E1A has been delivered and is functional (Yu et al., 1990). In a specific embodiment, there is upregulation of PEA15 expression level in cancer cells transfected with Ad.E1A(+). It is also assessed whether the extent of PEA15 upregulation depends on the E1A “dose” (titer).

### Determination of ERK Accumulation in the Cytoplasm of E1A-Overexpressing Cancer Cells

The localization of both ERK and phosphorylated ERK (pERK) in the cancer cells that constitutively express E1A is determined. In a specific embodiment of the present invention, when PEA15 is upregulated in such cells, ERK accumulates in the cytoplasm. It is determined if the localized ERK is an activated form (pERK), or if localization of pERK has a functional effect at the level of nuclear transcription. In one aspect of the invention, this is completed by studying a variety of exemplary cancer cell lines, such as human breast and ovarian cancer cell lines, to identify the generality of this effect.

For these experiments, the exemplary breast cancer (MDA-MB-231) and ovarian cancer (OVCAR3, SKOV3ip1) E1A stable transfectants are utilized. For the sake of brevity, the experiment by OVCAR3 (OV3)-E1A cells is described, but other cell lines are tested for the generality of phenomenon. Previous findings indicate that PEA 15 blocks the ability of ERK to phosphorylate the transcription factor Elk-1 and to promote ERK-dependent transcription (Formstecher et al., 2001; Gille et al., 1995). First, the cellular locations (i.e., in the cytoplasm or the nucleus) of ERK, pERK, and PEA15 are assessed by using confocal microscopy.

Then, it is determined if the location of ERK depends on the presence of PEA 15 by “removing” PEA 15 expression from cells transfected with E1A cells by using RNA interference (RNAi). RNAi involves the use of 21-nucleotide-long small interfering RNAs (siRNA) that bind to the target mRNA, degrading the mRNA and preventing expression of the target protein. siRNA can strongly suppress gene expression (Elbashir et al., 2001; Bernstein et al., 2001; Ueno et al., 1998) and can be more effective than antisense RNA for gene silencing (Bernstein et al., 2001). An anti-PEA15 siRNA is constructed from the sequence ggaagacatccccagcgaatt (SEQ ID NO:31) (Dharmacon Research; Lafayette, Colo.). Cells previously transfected with E1A are plated in 35-mm culture plates and grown to 30% to 50% confluence overnight. Then, the cationic liposome Oligofectamine (Invitrogen; Carlsbad, Calif.) is used to transfect the plated cells with 20 μM. antiPEA15 siRNA or 20 μM scrambled siRNA. Samples are collected for western blot analysis and cells are fixed for immunocytochemical analysis at 24 h, 48 h, 72 h, and 96 h. For the immunocytochemical studies, a polyclonal anti-PEA15 antibody, a polyclonal anti-ERK1/2 goat antibody (Santa Cruz Biotechnology; Santa Cruz, Calif.), and a monoclonal anti-pERK antibody (Cell Signaling; Beverly, Mass.) are used. Secondary antibodies will be rabbit-FITC (green), goat-Texas red (red), and mouse-Texas red (red). The nuclei are stained with topro-3 (blue). In a specific embodiment of the present invention, ERK and pERK are in the cytoplasm of cells transfected with the control siRNA (scrambled sequence) but in the nucleus of cells treated with anti-PEA15 siRNA. The levels of PEA15 may be reduced or completely abolished by 2-3 days after siRNA treatment.

The present inventors collect samples of cells treated with anti-PEA15 or scrambled siRNA for cytoplasmic and nuclear fractionation and western blotting with anti-pERK and anti-ERK antibodies. In a specific embodiment, these findings confirm the confocal microscopy findings with regard to cytoplasmic and nuclear accumulations of pERK and ERK.

To confirm that the activated form of pERK is truly sequestered and that this reduces ERK-dependent transcription, Elk-1 transcription is measured by using the Pathdetect in vivo signal transduction pathway Elk-1 transreporting kit (Stratagene, Lo Jolla, Calif.). In this kit, the Gal4-Elk1 plasmid contains the DNA-binding domain of GAL4 fused to the transactivation domain of Elk-1; the GAL4-luciferase construct has the luciferase reporter gene under the control of a synthetic promoter containing five tandem repeats of the yeast GAL4 binding sites. ELK-1 transcription is measured by the expression of active luciferase. In a specific embodiment, the luciferase activity (and hence the Elk-1-dependent transcriptional activity) is low in cells transfected with E1A (high PEA15) and high in cells transfected with the neopool control (low PEA15). This study is conducted in both the presence and absence of serum, as serum stimulation is known to activate the ERK pathway.

Thus, in a preferred embodiment of the present invention, it is shown that overexpression of E1A leads to sequestration of pERK (activated form) in the cytoplasm, which results in decreased transcription of Elk. MTT assays, [³H]-thymidine incorporation assays, western blotting, immunohistochemical staining, assessments of colony formation in soft agar, FACS analyses, adenoviral propagation and transfection, human cell cultures, and in vivo tumorigenicity studies utilized in this Example and others are all routine and have been used previously by the inventors (Ueno et al., 2000; Ueno et al., 2002; Ueno et al., 1997; Ueno et al., 1998). siRNA experiments have also been conducted successfully (see Example 3).

### Determine if PEA15 is Required for E1A's Anti-Tumor Effect

It is determined whether PEA15 upregulation contributes to the anti-tumor activity of E1A by knocking out PEA15 in OV3 ovarian cancer cells and several other cancer cell lines. These studies require that PEA15 is suppressed in E1A stable transfectants for long periods. Because the knockout effect of regular siRNA is typically limited to 7-10 days, a new vector system, pSUPER-RNAi (Oligoengine, Seattle, Wash.) is used, which directs the persistent synthesis of siRNAs in mammalian cells by integrating the pSUPER vector into target cell genome. This process involves cloning a 64-mer synthetic oligonucleotide that encodes two 19-nucleotide reverse complements that are homologous to a portion of the PEA15 target sequence separated by a short spacer region, which has no homology to either of the 19-nucleotide sequences. This oligonucleotide is cloned into the pSUPER vector into the Hind III and BglII sites. The hairpin is designed to be an optimal substrate for the enzyme dicer, which cleaves it. The pSUPER-PEA15 and pSUPER-Control constructs are cotransfected with puromycin at a ratio of 10:1 into cells previously transfected with E1A, producing the double stable transductants OV3-E1A-pSUPER-PEA15 and OV3-E1A-pSUPER-Control. The neomycin marker selects for E1A and puromycin will select for pSUPER-PEA15 and pSUPER-Control.

Because OV3-E1A cells are known to have high PEA15, low DNA synthesis, and low anchorage-independent growth, OV3-E1A-pSUPER-PEA15 clones and the OV3-E1A-pSUPER-Control (scrambled) clones are prepared. Because E1A was stably transfected with neomycin, hygromycin is used to select OV3-E1A clones with constitutively expressed siRNA-PEA 15. In a specific embodiment of the present invention, PEA 15 is knocked out in the cytoplasm despite the existence of E1A in this clone. These clones are tested for transformation (in soft agar assays) and for cell growth (by MTT and [³H]-thymidine incorporation assays). In another specific embodiment, there is an increase in DNA synthesis and an increase in the number of anchorage-independent colonies of the OV3-E1A-pSUPERPEA15 transfectants.

The tumorigenicity of these modified cells in nude mice is tested. Five groups of mice are used, with 10 mice/group. For these experiments, 107 viable cells in 0.5 ml PBS are injected under aseptic conditions into the intraperitoneal cavities of 4- to 6-week-old athymic female homozygous nu/nu mice; group 1 are injected with OV3 cells, group 2 with OV3-NP cells, group 3 with OV3-E1A cells, group 4 with OV3-E1A-pSUPERControl cells, and group 5 with OV3-E1A-pSUPERPEA15 cells. Tumor volume and body weight are measured periodically and survival duration and rate are monitored. The mice are examined for symptoms related to tumor growth such as ascites. Moribund mice are killed with an overdose of anesthesia and necropsy performed. Samples of tumors and other tissues are collected at necropsy and analyzed by immunohistochemical staining and western blotting for PEA15 expression. Proliferation is examined in terms of Ki-67 level. In a specific embodiment of the present invention, the mice in groups 3 and 4 survive the longest.

Thus, in a preferred embodiment of the present invention, PEA15 has a role in the anti-tumor activity of E1A in cancer cells.

### Example 8

### PEA15 Has Anti-Tumor Activity

As described elsewhere herein, PEA15 suppressed colony formation in exemplary ovarian cancer cell lines (SKOV3ip1, OVACAR3) and a breast cancer cell line (MDA-MB-435). PEA15 also suppressed the anchorage-dependent growth of SKOV3ip1 cells. In the studies provided below, it is further characterized that PEA15 functions as a tumor suppressor gene.

### Determination of the In Vitro Anti-Tumor Activity of PEA15 in Cancer Cells by Establishing Stable Transfectants

First, cancer cell lines, such as breast and ovarian cancer cell lines, are established that are stably transfected with PEA15 and thus express PEA15 constitutively. In an exemplary embodiment, breast and ovarian cancer cells (SKOV3ip1, OVACAR3, 2774, MDA-MB-435, and MDA-MB-231) are transfected with pcDNA3-HAPEA1 and the resulting colonies screened for increased expression of PEA15. Specifically, cells are transfected with 2 μg of vector (pcDNA3, which encodes only the neomycin resistance gene) or 2 μg of pcDNA3-HAPEA15 (which encodes hemagglutinin-tagged PEA15 cDNA) by using liposomes to deliver the DNA into the cells. At 48 hours after transfection, cells are trypsinized and replated in 100 mm plates at a 1:5 dilution. The transfectants are selected in medium containing 50.0 μg/ml of G418 for 3 weeks, after which individual neomycin-resistant colonies are cloned and expanded to mass culture. The PEA15 stable transfectants are screened by western blotting analysis to identify colonies with increased expression of PEA15. Three PEA15 stable transfectants from each cell line, together with the corresponding parental and vector (neo pool) controls, are selected for analysis.

### Studies for the PEA15 Stable Transfectants are Summarized Below.

i. Suppression of proliferation and tumorigenicity by PEA15. In comparisons of PEA15 expression level and the basic phenotype of the stable transfectants, in a specific embodiment there is less proliferation and/or less tumorigenicity in cells with high expression of PEA15. Growth rate is examined with MTT assay; proliferation is measured in terms of DNA synthesis with a thymidine-incorporation assay; and anchorage-independent growth-generally considered a marker of transformation, which correlates well with tumorigenicity-is assessed in soft agar. In a specific embodiment, the PEA15 stable transfectants form fewer colonies in soft agar than the parental and neopool controls. Further, apoptosis is examined by FACS and TUNEL assay after depletion. It is recognized that FasL and TNF-α induced apoptosis can be inhibited by PEA-15 but a different type of external environment (serum depletion and DNA damaging agents) may result in induction of apoptosis.

ii. Identifying molecules targeted by PEA15 to reduce cell proliferation. The pattern of gene expression of PEA15 in stable transfectants and parental cells is examined as follows. RNA is extracted from stable transfectants by using the RNAeasy kit (Qiagen Inc., Valencia Calif.). Gene expression array analysis is performed with the Affymetric system (Santa Clara, Calif.) and the U95 A-E microarray chip, which contains oligonucleotides from 41,441 known genes, and eSTS is used. Microarray data are analyzed with the D-Chip program. This microarray analysis is supported by microarray core laboratory at our institution. There is comparison of at least three pairs of PEA15 stable transfectants to select candidate genes that seem to have similar changes. Those genes are identified that are upregulated or downregulated by 2.5 times or more in any analysis. Groups are compared with Fisher's exact test. RT-PCR is used to confirm the change in gene expression using RNA from the stable transfectants. If relevant antibodies are available commercially or from our collaborators, protein expression is studied, although standard means in the art may be utilized to generate them. After the potential genes are identified, they are studied to determine whether they are required for suppression of cell proliferation by PEA15 through experiments similar to those used in Example 7.

iii. In vivo tumorigenicity studies are then be conducted in which the transfected cells are injected into the mammary fat pad (breast cancer cells) or the peritoneum (ovarian cancer cells) of nude mice, as described below. In a specific embodiment, mice injected with the PEA 15 stable transfectants have smaller tumors and longer survival.

Tumorigenicity assays for cancer, such as the exemplary breast cancer. Cells in log-phase growth are trypsinized, washed twice with PBS, and centrifuged at 1000×g. Viable cells are counted, and 1×10⁶ cells in 0.2 ml of PBS are injected into the mammary fat pads of female nude mice under aseptic conditions. Survival duration is tracked and tumor volume calculated at necropsy. Mice (10) are then injected with PEA-stable breast cancer transfectants and another 10 mice with a control cell line. Tumor volumes are estimated as the product of caliper measurements in 3 dimensions.

Tumorigenicity assays for ovarian cancer. Cells in log-phase growth are trypsinized, washed twice with PBS, and centrifuged at 1000×g. Viable cells are counted and 2×10⁶ cells in 0.5 ml of PBS are injected under aseptic conditions into the peritoneal cavities of female nude mice. Ten mice are injected with PEA-stable ovarian cancer transfectants and another 10 mice with a control cell line. Survival data are summarized and plotted using the method of Kaplan and Meier; the resulting survival curves are compared using log-rank tests.

### Examine the Therapeutic Effects of PEA15 in Human Cancer Xenograft Models

An animal study to characterize the action of PEA15 as a tumor suppressor gene in vivo is performed. The exemplary animal models to be used are described further below.

i. Ovarian cancer xenograft treatment model. A preclinical animal model is developed involving the cationic liposome DC-Chol gene delivery system. Because this system has already been shown to be safe and effective for local transfection in human subjects, it is appropriate to use in models of ovarian cancer as well. The ovarian cancer cell lines SKOV3-ip1 and OVACAR3 readily produce tumors in nude mice. For these studies, cells growing at log-phase are trypsinized, washed twice with PBS, and collected by centrifugation at 250×g. Viable cells are counted, and 2×10⁶ viable SKOV3-ip1 cells (in 0.5 ml PBS) or 10×10⁶ viable OVACAR3 cells (in 0.5 ml PBS) are injected under aseptic conditions into the intraperitoneal cavities of 4- to 6-week-old athymic female homozygous nu/nu mice. There are, for example, four groups of 10 mice each. Five days after being injected with the ovarian cancer cells, the mice are treated as follows: group 1 is the control (untreated), group 2 is given liposome, group 3 is given vector liposome, and group 4 is given the PEA15-liposome complex. Treatments are given by weekly intraperitoneal injections for 12 weeks. Mice are monitored daily for tumor volume, body weight, and the presence of symptoms related to tumor growth such as ascites. Mice that seem moribund are killed with an overdose of anesthesia and necropsy performed. Samples of tumors and other tissues are collected at necropsy and analyzed by immunohistochemical staining and western blotting for PEA15 expression, cytoplasmic (as opposed to nuclear) location of ERK and pERK, and the proliferation marker Ki-67 (Novocastra Lab Ltd; Newcastle Upon Tyne, UK). In a specific embodiment of the present invention, there is slower proliferation in cells from the mice treated with the PEA15-lipsome complex. If no reduction in proliferation is observed, the extent of apoptosis is studied by TUNEL assay of tumor cells from these animals. Survival is analyzed according to the method of Kaplan and Meier. In a preferred embodiment of the present invention, PEA15 inhibits ovarian tumorigenicity to a greater extent than that in the control groups.

ii. Optimal conditions for efficient transfection of a reporter gene by LPD cationic liposomes. LPD liposomes are developed for use as a systemic gene delivery system. LPD liposomes have been used to transfer genes in a breast cancer xenograft model (see Example 6), but delivery conditions for this system may be optimized. Because advanced cancer is a systemic disease, LPD is more suitable than DC-Chol, which is preferable for local gene delivery. It is examined whether genes can be delivered systemically to xenografted tumors in the peritoneum (ovarian cancer model) or in the mammary fat pad (breast cancer model) of nude mice, as exemplary models.

Given intravenously, LPDs can deliver a gene systemically to normal organs although some optimization of gene expression in tumors. Previous studies have focused on the activity of reporter genes (luciferase or CAT) in normal organs such as liver, lung, and kidney after a one-time tail-vein injection of a reporter gene complexed with LPD. A repetitive injection schedule that allows continuous, systemic gene expression of PEA 15 may be utilized.

An optimal dose and timing for achieving high transfection efficiency and expression of PEA15 in breast cancer and ovarian cancer cells is determined. To this end, a luciferase gene complexed with LPD (luc-LPD) is injected via the tail vein into mice bearing human breast cancer or ovarian cancer xenografts. Eight treatment groups of 10 mice each are used (four for each type of cancer). Treatments are given as follows: group 1 is injected with PBS, group 2 with luciferase, group 3 with LPD, and group 4 with luc-LPD. The LPD and DNA concentrations are titrated to obtain the highest transfection efficiency in vivo without inducing precipitation of the luc-LPD complex in vitro. Luciferase activity is measured at four time points (24 hours, 72 hours, 1 week, and 2 weeks) to determine the kinetics of luciferase expression. Then, depending on the half-life of the luciferase protein, it is determined how often the injection should be repeated to ensure continuous expression of luciferase. In this regard, it is already known that the LPD-DNA complex can induce the transient expression of cytokines such as 7-interferon and TNF-α, which in turn suppresses the transfection efficiency of a second LPD-DNA transfection, and that this effect may last as long as 7-14 days (Whitmore et al., 1999; Whitmore et al., 2001). The optimal interval for repeated injections to achieve maximum expression is therefore determined. Initially, it is assumed that 2 weeks is the optimal interval for repeated injection. Luciferase activity in tumors are also be compared with that in other organs such as the lungs, which were the most commonly transfected organs when luc-LPD complex was injected into mice via the tail vein (Li and Huang, 1997; Li et al., 1998). The above-mentioned studies are repeated using β-galactosidase (or enhanced green fluorescent protein) as exemplary reporter genes to allow visual quantification of the extent of gene expression in the tumors.

iii. Systemic delivery of the PEA15 gene in a breast cancer xenograft model. Once the optimal protocol for gene expression has been established as described above, the luciferase gene is replaced with the PEA15 gene and the construct is complexed with LPD. The control is an empty vector that cannot express PEA15. The PEA15-LPD complex is then administered via tail-vein injection in an orthotopic model of breast cancer in mice. Overall anti-tumor activity is determined in terms of animal survival and tumor size. Pathologic changes are also examined in normal tissue to assess toxicity. In a preferred embodiment of the present invention, PEA15 reduces tumor size and prolong survival as compared with empty-vector treatment.

For these experiments, MDA-MB-435 breast cancer cells growing at log phase are trypsinized, washed twice with PBS, and collected by centrifugation at 250×g. MDA-MB-435 is selected here on the basis of results described in Example 4, although any cell line that shows suppressed anchorage-dependent growth by PEA can be used. Viable cells will be counted and 2×10⁶ viable cells in 0.5 ml PBS is injected under aseptic conditions into the mammary fat pads of 4- to 6-week-old athymic female homozygous nu/nu mice. When tumors grow to at least 0.5 mm in diameter, treatment begins. Four groups of 10 mice each are used: group 1 is treated with PBS, group 2 with empty vector-LPD, group 3 with PEA15 alone, and group 4 with PEA15-LPD. Injections are made through the tail veins, at intervals and doses identified in the previous experiment (section D2bii) as being optimal for sustained gene expression. Mice are monitored daily for tumor growth and morbidity; tumor size is determined at necropsy. PEA15 expression and cell proliferation (Ki-67) is measured in representative tumor tissues by immunohistochemical staining. Overall survival is assessed by the Kaplan-Meier method and group differences is assessed by the log-rank test. Other experiments are analyzed with Student's t test. In a preferred embodiment of the present invention, PEA15 prolongs the life span of these animals. Similar experiments are conducted with the ovarian cancer xenograft model, based on the optimal gene delivery system described elsewhere herein.

### Example 9

### The Effects of PEA15 on the Sensitivity of Cancer Cells to Chemotherapeutic Agent

In a specific embodiment of the present invention, PEA15 sensitizes cells cancer cells to paclitaxel by inactivating the ERK pathway. The combination of paclitaxel with PEA15 gene therapy represents a novel molecular, chemotherapeutic strategy. As noted in Example 5, paclitaxel sensitivity of human ovarian cancer cells is enhanced when PEA15 is constitutively expressed.

Many anticancer agents can induce apoptosis, but resistance to anticancer agents is a major hindrance to their use. Paclitaxel activates the prosurvival MEK/ERK pathway, which may affect the efficiency of paclitaxel in inducing apoptosis (McDaid and Horwitz, 2001; MacKeigan et al., 2000). Paclitaxel is only an exemplary drug for testing the sensitivity provided to a cancer cell from PEA15, so others may be utilized in an analogous manner as described herein. It is also known that PEA15 sequesters ERK in the cytoplasm, preventing the phosphorylation of its nuclear substrate Elk and resulting in reduced cell proliferation. Therefore, an enhancement of apoptosis in cancer cells when a combination of paclitaxel and PEA15 are used is one aspect of this invention. This combination has a synergistic effect, in a specific embodiment, as PEA15 binds to ERK that is induced by paclitaxel. Thus, the combination of PEA15 gene therapy and the exemplary paclitaxel is an effective therapeutic strategy for breast cancer and ovarian cancer.

### In Vitro Sensitization to Paclitaxel by PEA15 Expression in Ovarian and Breast Cancer Cells

i. Using PEA15 stable transfectants to test paclitaxel sensitivity. For the following studies, the PEA15 stable transfectants described elsewhere herein are used to test their chemosensitivity to paclitaxel. Parental and PEA15 stable transfectants will be treated with 0.001 μM to 10 μM paclitaxel. IC₅₀ values are calculated by testing over a 3- to 5-log range with 10-fold dilutions to include the therapeutic dose range. Cell viability is measured (by MTT assay) at 24 h, 48 h, and 72 h after treatment. In a specific embodiment, the IC₅₀ is significantly lower for the PEA 15 stable transfectants than for the parental cells.

The expected increase in cytotoxicity from using paclitaxel with PEA15-expressing cells is then examined to determine if this effect resulted from increased apoptosis. Apoptosis is measured by FACS analysis and TUNEL assay. The anchorage-independent growth in soft agar of the PEA15 transfectants treated with paclitaxel is also assessed. In a specific embodiment of the present invention, the PEA15 stable transfectants treated with paclitaxel to form fewer colonies than the parental cells treated with paclitaxel. FACS analysis and TUNEL assay has been done previously by the inventors (Ueno et al., 1997).

ii. Using adenovirally encoded PEA15 to test paclitaxel sensitivity. To rule out the possibility that use of stable transfectants results in clonal selection, paclitaxel sensitization is examined in cells transfected with PEA15 with, for example, a vector, such as an adenoviral vector. The exemplary adenoviral vector is constructed that contains cDNA of PEA15 (Ad.PEA15(+)). Initially, the most appropriate virus/tumor cell ratio is chosen on the basis of the highest transduction efficacy (>80%) and the lowest toxicity for each individual cancer cell line. The exemplary ovarian cancer cell lines (e.g., OVACAR3, SKOV3-ip1, 2774) and breast cancer cell lines (e.g., SK-Br-3, MDA-MB-453, MDA-MB-435, MDA-MB-231) are treated with Ad.PEA15(+) or Ad.PEA15(−) vectors on day 0. If the PEA15 expression pattern is established as expected, paclitaxel is added, and the cells are incubated for several days and processed for various assays (e.g., MTT, [3H] thymidine incorporation, cell counts, or colony formation). To calculate the IC₅₀ values, paclitaxel is tested over a 3- to 7-log range, using 10-fold dilutions, to cover the entire dose-response curve. In a specific embodiment, the IC₅₀ for PEA15-overexpressing cells is significantly lower for the Ad.PEA15(+)-treated cells than for Ad.PEA15(−)-treated cells.

iii. Suppression of Elk-1 activity by PEA15 and enhancement of paclitaxel-induced apoptosis. The impact of PEA15 on ERK-dependent transcription is measured by measuring Elk-1 transcription in the PEA15 transfectants treated with paclitaxel. In a preferred embodiment of the present invention, Elk-1 activity is low in these stable transfectants because of the sequestration of ERK by PEA15 in the cytoplasm. PEA15 stable transfectants are rescued by transfecting them with Elk-1 and then treat the cells with paclitaxel. In a preferred embodiment of the present invention, Elk-1 transfection in the PEA15 transfectants rescues (suppresses) the enhanced paclitaxel-induced apoptosis. The pSUPER-PEA15 (siRNA PEA15 expression vector) described above is used to knock out the PEA15 in PEA15 stable transfectants and the paclitaxel sensitivity of those cells is compared to that of the PEA15 stable transfectants. In a preferred embodiment of the present invention, the PEA15-knockout stable transfectants are more resistant to paclitaxel than the PEA 15 stable transfectants. Elk-1 activity in the knockout cells is measured, and in a preferred embodiment that activity is higher in the knockout cells than in the PEA15 stable transfectants.

In a preferred embodiment of the present invention, the combination of PEA15 and paclitaxel is more effective than either modality alone, as PEA15 inhibits the survival signal ERK and makes the cells more sensitive to paclitaxel. It is known that combining MEK/ERK inhibitors with paclitaxel enhanced tumor apoptosis in ovarian, breast, and lung tumor cell lines (99, 100).

### Combined PEA15 and Paclitaxel in Cancer Models

i. Enhancing paclitaxel-induced cell death by combining paclitaxel and PEA15 gene delivery in an exemplary cancer model, such as an ovarian cancer xenograft model. The exemplary ovarian cancer xenograft model developed for PEA-DC-Chol described above is utilized herein. Briefly, ovarian cancer cells are injected under aseptic conditions into intraperitoneal cavities of 4- to 6-week-old athymic female homozygous nu/nu mice, and 5 days later, treatment is begun. Five treatment groups, with 10 mice each, is used as follows: group 1 is the control (PBS), group 2 is given empty vector-DCC-Chol liposome, group 3 is given PEA15-liposome (15 μg of PEA15). These injections are given intraperitoneally once a week for 12 weeks. Mice in group 4 are given paclitaxel as intraperitoneal injections every 3 weeks, and mice in group 5 are given PEA15-liposome and paclitaxel. Mice are monitored daily for tumor volume, body weight, and the presence of symptoms related to tumor growth such as ascites. Mice that seem moribund are killed with an overdose of anesthesia, and necropsy is performed. Samples of tumors and other tissues are collected at necropsy and analyzed by immunohistochemical staining and western blotting to confirm PEA15 expression, cytoplasmic (as opposed to nuclear) location of pERK, and Ki-67. Survival is analyzed using the method of Kaplan and Meier. In a preferred embodiment of the present invention, the mice treated with PEA 15 and paclitaxel survive longer than the mice treated with either modality alone. Log-rank analysis is used to compare survival between groups.

ii. Enhancing paclitaxel-induced cell death by combining paclitaxel and systemic PEA15 gene delivery in an exemplary cancer model, such as a breast cancer xenograft model. The breast cancer xenograft model developed for PEA15-LPD described above is used for this study. Mice are exposed to paclitaxel at concentrations that cannot control the tumor to allow determination whether PEA15 could enhance the efficiency of paclitaxel. Overall anti-tumor activity is determined in terms of animal survival and tumor size, and toxicity by pathology analysis of normal tissues. In a preferred embodiment of the present invention, the combination of PEA15 and paclitaxel reduces tumor size and prolongs survival as compared with treatment with either modality alone.

Briefly, five treatment groups, each containing 15 mice, are used: group 1, PBS; group 2, PEA15-LPD; group 3, paclitaxel; group 4, empty vector-LPD plus paclitaxel; and group 5, PEA15-LPD plus paclitaxel. Before treatments are begun, MDA-MB-435 breast cancer cells are injected under aseptic conditions into the mammary fat pads of 4- to 6-week-old athymic female homozygous nu/nu mice. When tumors grow to >0.5 mm in diameter, PEA15-LPD (or the other liposome controls) is injected via the tail vein, and chemotherapy is given by intraperitoneal injection on the third day after the liposome injection. Animal survival and tumor size is measured. PEA15 expression and cell proliferation (Ki-67) is also measured in representative tumor tissues by immunohistochemical staining, and cell death is assessed by TUNEL assay. In a specific embodiment, the chemotherapy dose is based on the LD₅₀ (the dose lethal to 50% of the mice). The total number of mice needed to demonstrate a statistically significant (30%) reduction in death induced by tumor is also calculated. Overall survival is assessed by the Kaplan-Meier method, and group differences are assessed with log-rank tests. Other comparisons will be analyzed with Student's t test.

### Example 10

### E1A Inhibits the Tumorigenicity of Low-HER2-Expressing Ovarian Cancer Cells

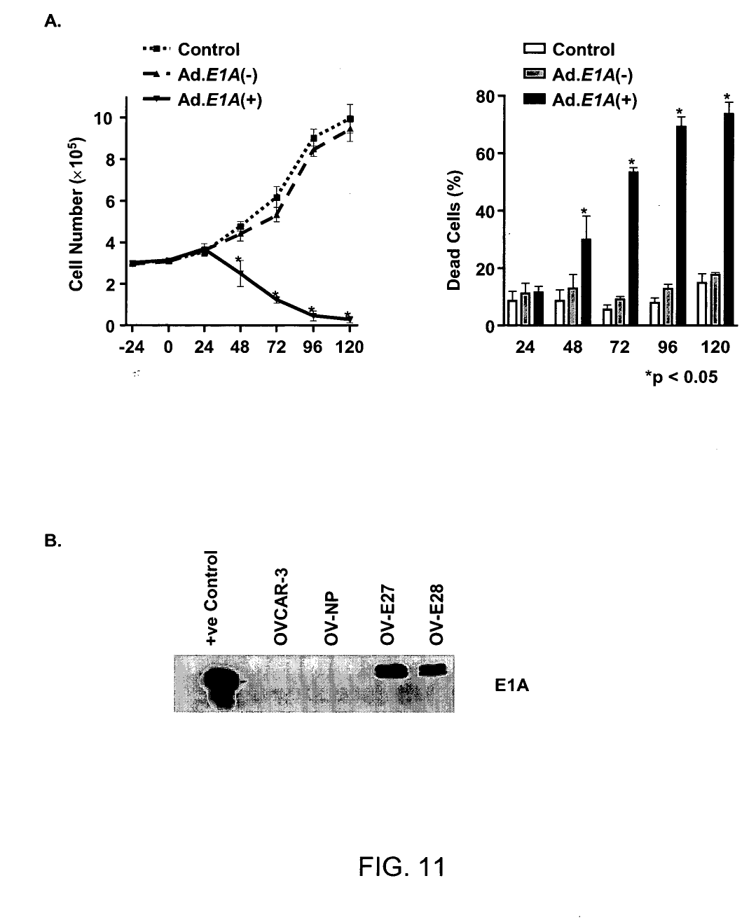

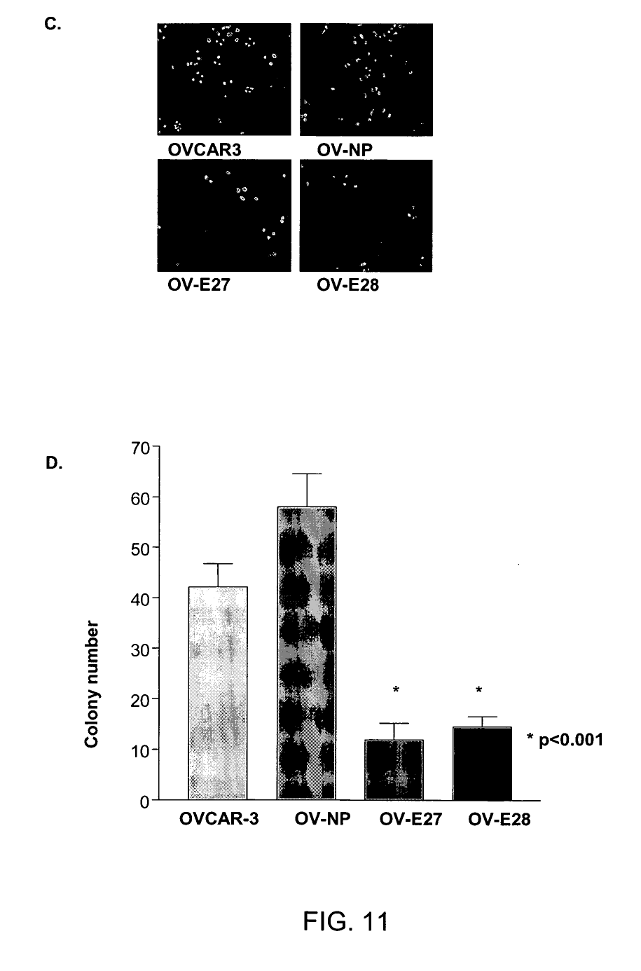

Samples obtained from a previous human clinical trial of E1A gene therapy showed reduced levels of Ki-67 (reflecting inhibition of cell proliferation) in cancer cells from E1A-treated patients regardless of their HER2 expression levels (Hortobagyi et al., 2001). Thus, we first carried out an in vitro study of OVCAR-3 cells, previously shown to respond to transfection with Ad.E1A by suppression of tumorigenicity, to determine if Ad.E1A(+) could inhibit cell proliferation in vitro. The present inventors found that infecting OVCAR-3 cells with Ad.E1A(+) reduced their growth rate to 5.3% of that of cells expressing a mock vector at 96 h (P <0.05) and that this reduction correlated with an increase in cell death (FIG. 11A).

OVCAR-3 cell lines were generated that constitutively express E1A (FIG. 11B). A reduction in DNA synthesis was confirmed, another hallmark of reduced cell proliferation, in the OVCAR-E1A cells relative to that in the parental cells, as indicated by BrdU incorporation (FIG. 11C). The present inventors also tested their ability to form colonies in soft agar, an indicator of in vivo tumorigenicity. The two OVCAR-E1A clones tested [OVCAR-E27 and OVCAR-E28] formed 64% fewer colonies in soft agar than did control cells [parental OVCAR-3 or OVCAR-NP cells] (P<0.0001) (FIG. 11D).

### Example 11

### PEA15 is Upregulated in OVCAR-E1A Cells and Sequesters ERK in the Cytoplasm

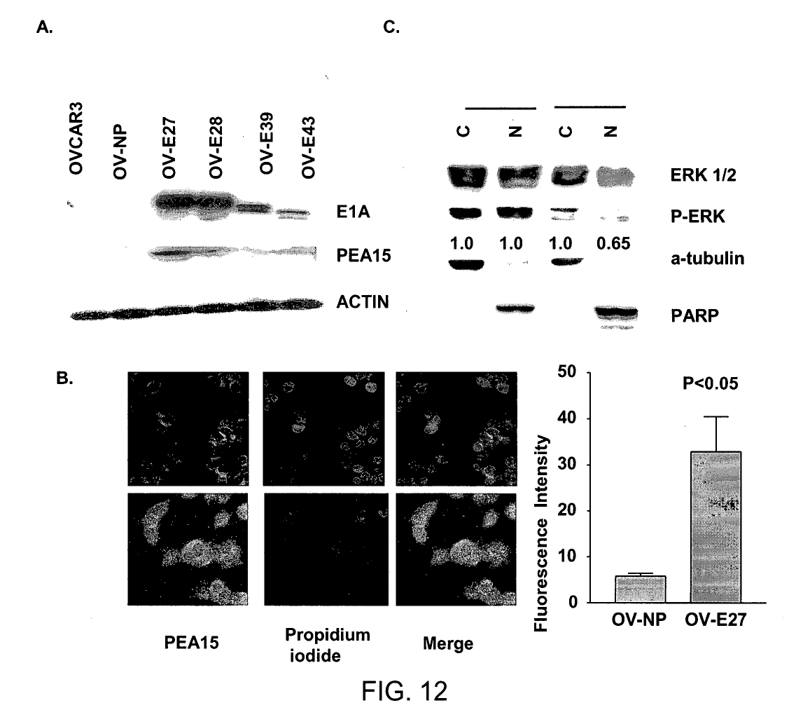

To identify targets of E1A that mediate its inhibitory effect on cell proliferation, the present inventors compared the gene expression profiles of the parental OVCAR-NP cells and OVCAR-3-E1A cells. They initially identified many genes that were differentially expressed between the parental cells and the E1A-expressing cells. Differential expression of those candidate genes in terms of their protein levels were confirmed. Among the candidate gene products, PEA15 was found to be upregulated in the OVCAR-E1A transfectants as compared with control cells [OVCAR-3 or OVCAR-NP] by western blot analysis (FIG. 12A). Immunofluorescence staining also showed that PEA15 protein was more highly expressed and tended to be in the cytoplasm (rather than in the nucleus) in OVCAR-E1A cells in comparison with the OVCAR-NP controls (FIG. 12B). PEA15 was also upregulated in the ovarian cancer cell line SKOV3-ip1 stably transfected with E1A. These findings indicate that E1A expression is involved in the upregulation of PEA15.

### Example 12

### PEA15 and ERK

PEA15, a multifunctional protein that regulates both apoptosis and cell proliferation (Renault et al., 2003), has been found to relocate ERK from the nucleus to the cytoplasm (Formstecher et al., 2001; Whitehurst et al., 2004; Renault et al., 2003), leading to suppression of proliferation. The location of ERK and phosphorylated (activated) ERK (PERK) in the E1A-expressing cells was assessed. Subcellular fractionation studies revealed that ERK and pERK were largely confined to the cytoplasm in the OVCAR-E1A transfectants relative to the vector control OVCAR-NP (FIG. 12C). Thus high levels of PEA15 in OVCAR-E1A cells were associated with ERK and pERK being sequestered in the cytoplasm.

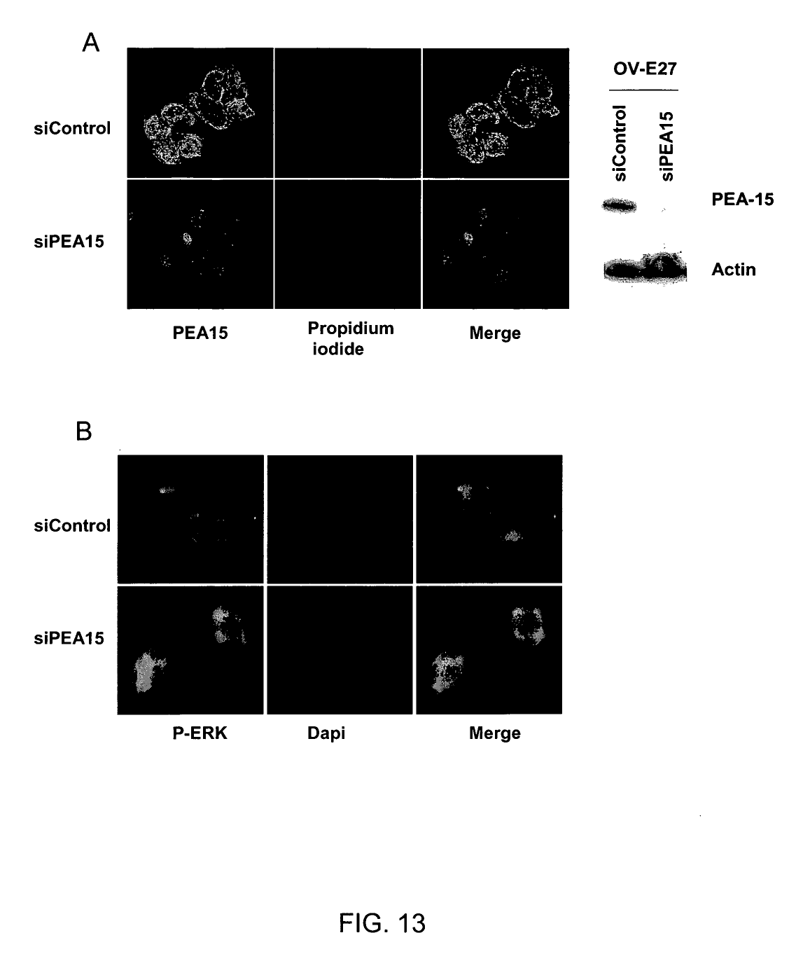

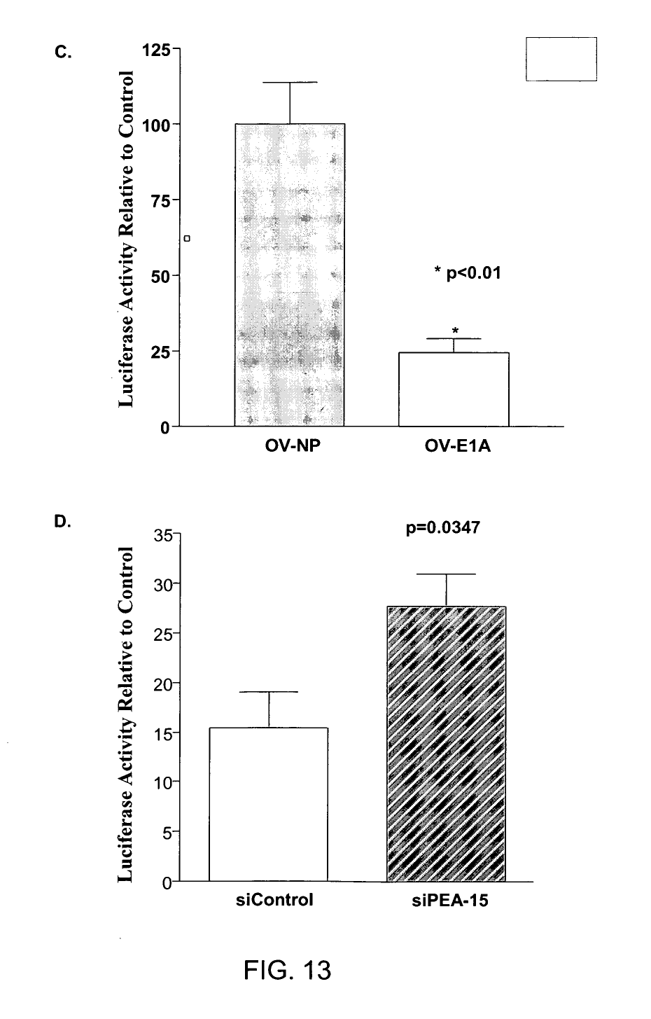

To examine if the location of pERK (nuclear or cytoplasmic) depends on PEA15, the present inventors next suppressed PEA15 expression by using siRNA. Transfection of OVCAR-E1A cells with siRNA specific for PEA15 sharply reduced the levels of PEA15 protein (FIG. 13A) and led to accumulation of pERK in the nuclei of those cells (FIG. 13B). To determine whether the nuclear location of ERK affected ERK activity, we measured the transcriptional activity of Elk, which is known to be activated by pERK. Elk activity was indeed low in the OVCAR-E1A cells relative to that in the control OVCAR-NP cells (FIG. 3C), and depletion of PEA15 by siRNA restored the Elk-1 activity in the OVCAR-E1A cells (FIG. 13D). Collectively, these findings suggest that ERK activity is regulated by the presence of PEA 15.

### Example 13

### The Antitumor Effect of E1A in Ovarian Cancer Cells Depends on PEA15

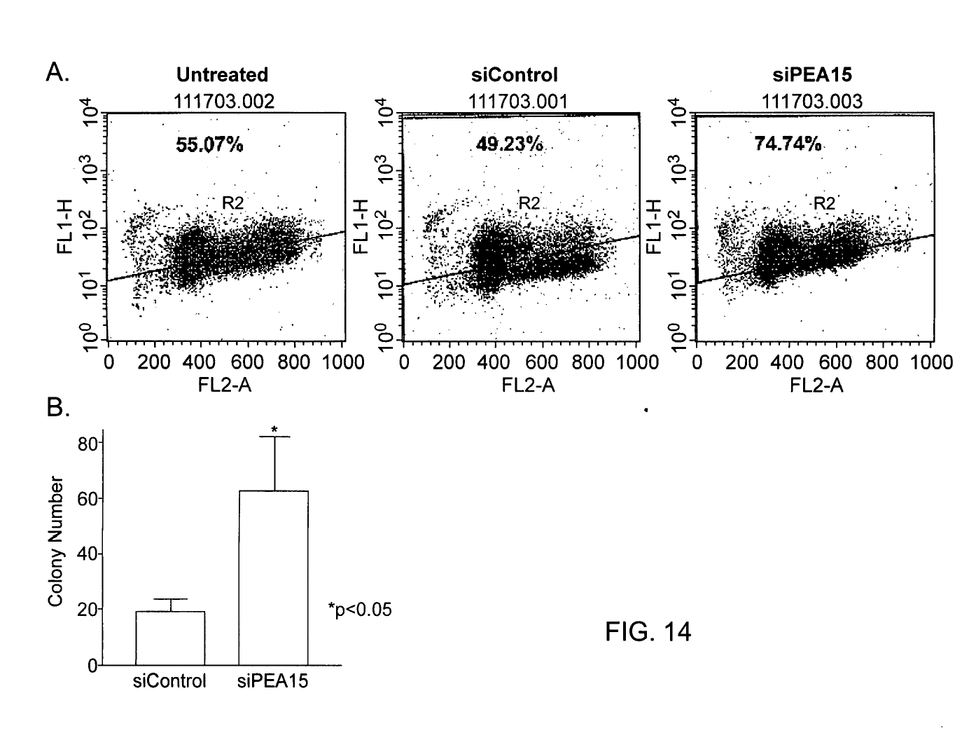

It was next determined whether the observed E1A-induced decrease in DNA synthesis in the OVCAR-E1A cells depends on the presence of PEA15. To do so, OVCAR-E1A cells were transfected with either siPEA15 or siControl, incubated at 37° C. for 30 h and then incubated with BrdU for a further 20 h; BrdU incorporation by FACS analysis was assessed. Indeed, the proportion of cells incorporating BrdU (i.e., proliferating) was higher in the OVCAR-E1A cells treated with siPEA15 (75%) than in siControl-treated cells (49%) or untreated cells (55%) (FIG. 14A).

It was also examined whether depletion of PEA15 would facilitate anchorage-independent growth of the OVCAR-E1A cells. The mean number of colonies in the siPEA15-treated E1A stable transfectants (63%) was three times as high as that of the siControl-treated cells (19%) (P<0.05) (FIG. 14B), suggesting that silencing PEA15 led to promotion of cell growth through increasing DNA synthesis and anchorage-independent growth. Hence in OVCAR-3 ovarian cancer cells, the antitumor activity of E1A depends on PEA15.

### Example 14

### PEA15 Inhibits Human Ovarian Cancer Cell Growth In Vitro

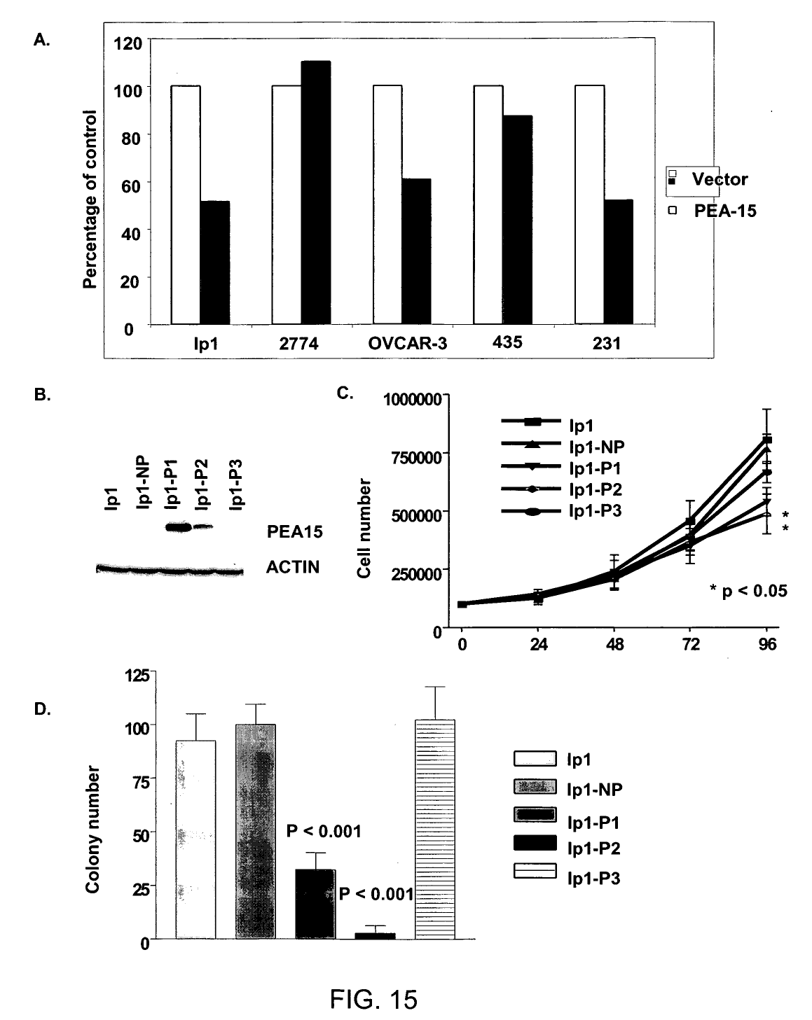

Finally, to determine whether PEA15 itself could suppress tumorigenicity in vitro, the present inventors transfected three ovarian cancer (SKOV3.ip1, 2774-CIO, and OVCAR-3) and two breast cancer (MDA-MB-435 and MDA-MB-231) cell lines with a vector control (pcDNA3) or with HA-PEA15 (HA-PEA15 cloned into pcDNA3) and assessed cell growth and colony-forming ability. Fewer G418-resistant colonies were found in the PEA15 transfectants (relative to the vector controls) in the SKOV3-ip1, OVCAR-3, and MDA-MB-231 cell lines (FIG. 15A), suggesting that PEA15 has a growth inhibitory effect on these cells. As a final test of the putative link between PEA15 expression and suppression of tumorigenicity, we established stable SKOV3.1ip1-PEA15 transfectant cells and screened the G418-resistant colonies for PEA15 expression by western blot analysis. Of the three clones obtained, ip1-P1 expressed the greatest amount of PEA15 protein (FIG. 15B). Clones 1 and 2 both grew at slower rates than the parental cells, vector-control cells, or a third clone (which expressed little to no PEA15) (FIG. 15C), which could be related to the difference in PEA15 protein levels. This apparent growth inhibitory effect of PEA 15 was confirmed in an anchorage-independent growth assay, in which clones 1 and 2 produced fewer colonies than the vector control (FIG. 15D) but the low-PEA15-expressing clone 3 showed no reduction in number of colonies compared with the vector control. Collectively, these results demonstrate that PEA15 suppresses the growth of ovarian cancer in vitro.

### Example 14

### PEA15 Inhibits Human Breast and Ovarian Cancer Cell Growth In Vitro

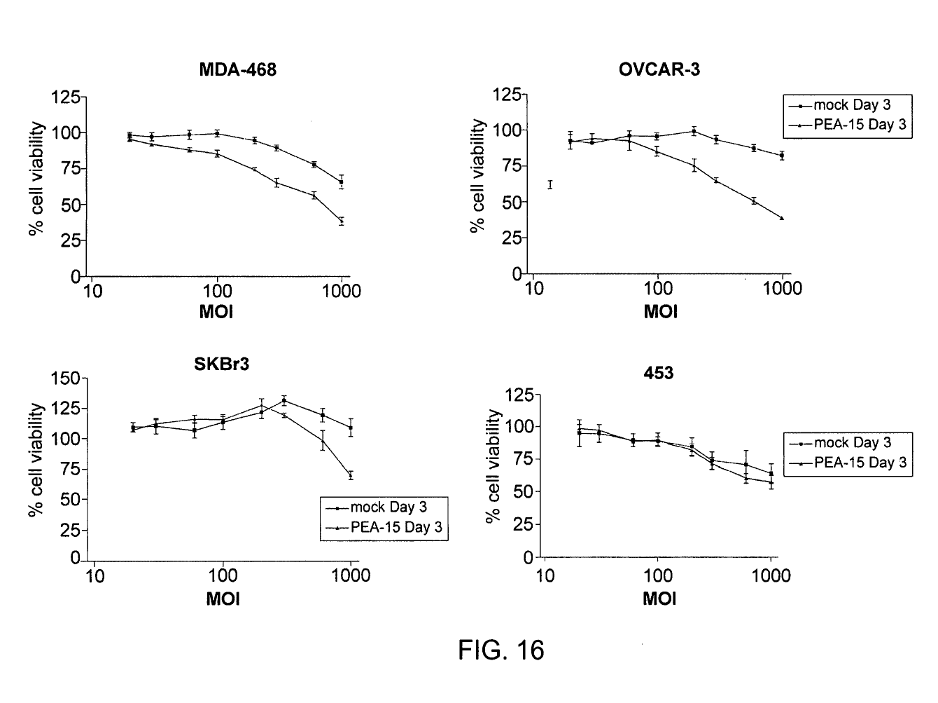

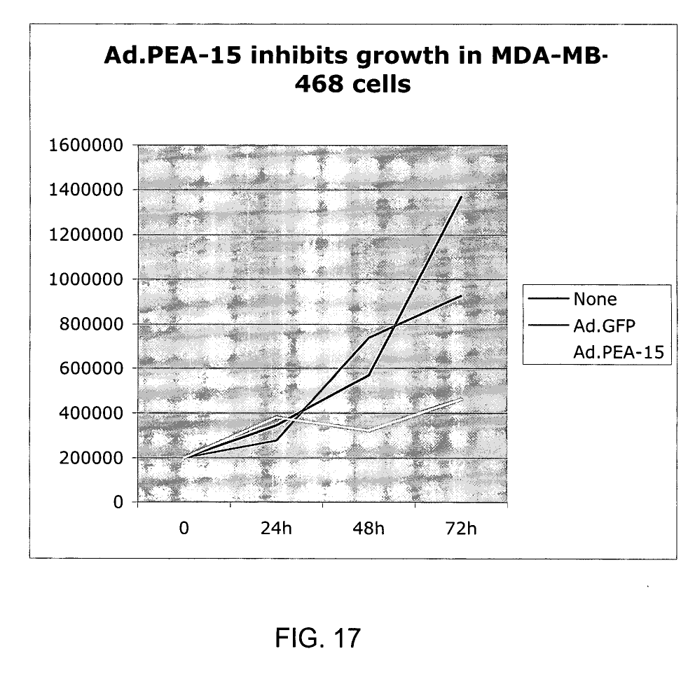

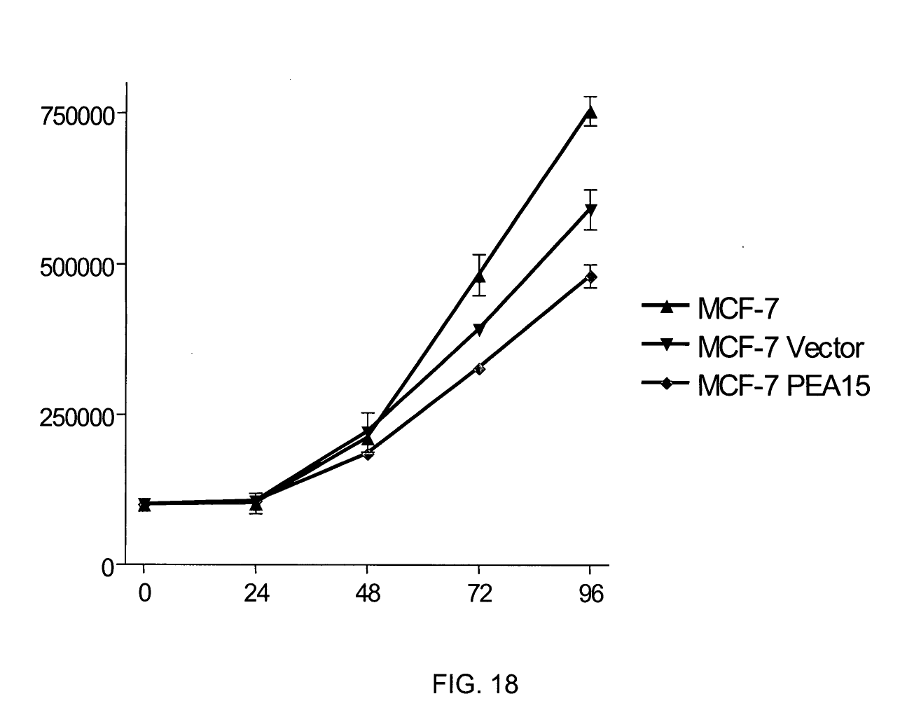

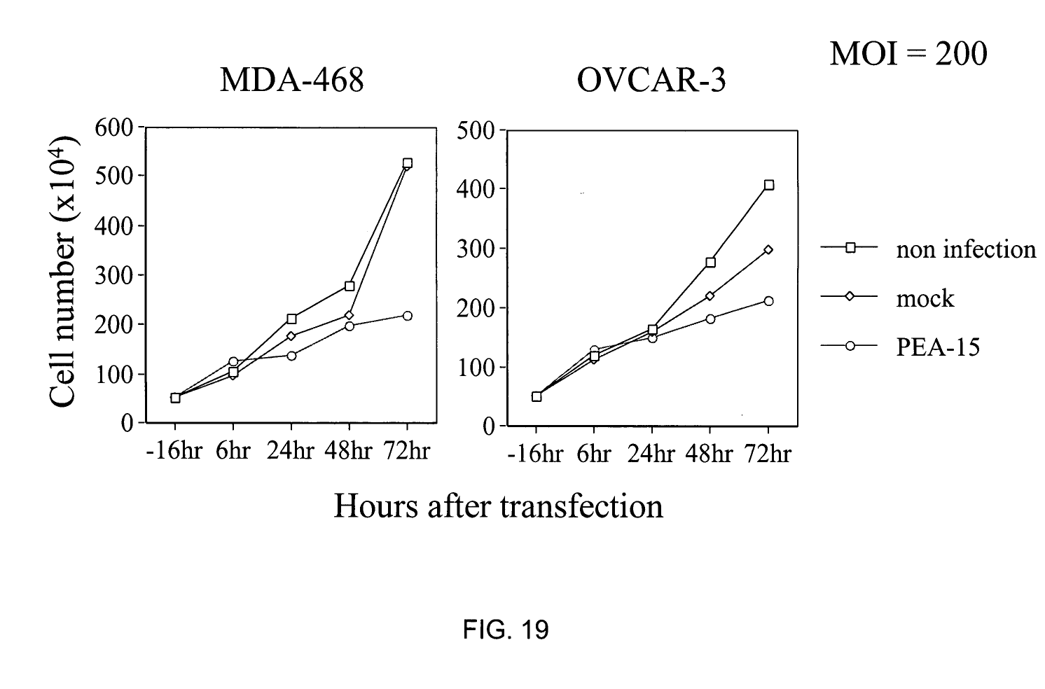

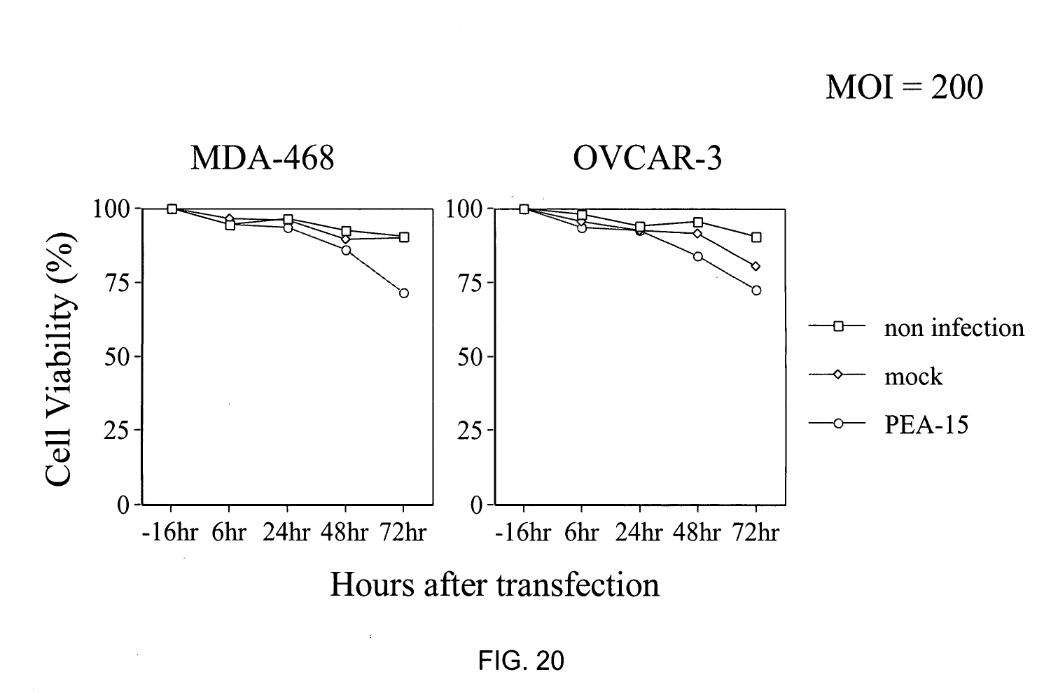

The present invention provides PEA15 compositions that inhibit human breast and ovarian cancer. FIGS. 16-20 show an exemplary adenoviral PEA15 composition that inhibits growth in some exemplary breast and ovarian cancer cells (at least some of which are illustrated via MTT cytotoxicity assay standard in the art).

### Example 16

### Exemplary Materials AND Methods

VII. Cell Lines and Culture Conditions

SKOV3.ip1 ovarian cancer cells were kindly provided by Dr. Mien-Chie Hung of The University of Texas M. D. Anderson Cancer Center (Yu et al., 1993). All other human breast and ovarian cancer cell lines were obtained from the American Type Culture Collection (Manassas, Va.). SKOV3.ip1 cells express high levels of HER2, whereas OVCAR-3 ovarian cancer cells (Xu et al., 1999) and MDA-MB-435 and MDA-MB-231 breast cancer cells express only basal levels of HER2 (Ueno et al., 1997). SKOV3.ip1, MDA-MB-435, and MDA-MB-231 cells were grown in DMEM/F12 medium (GIBCO, Grand Island, N.Y.), and OVCAR-3 cells were grown in RPMI 1640 (GIBCO) supplemented with 2 mM L-glutamine, 10% fetal bovine serum (FBS), and penicillin/streptomycin in a humidified incubator at 37° C. with 5% CO₂.

VIII. Transfection Conditions

For E1A transfections, two stably transfected cell lines, OVCAR-3-Neo and OVCAR-3-E1A, were established by transfecting 10 μg of either a control vector (pSV2-neo) (BD Biosciences, Palo Alto, Calif.) or pSV2 expressing the adenovirus type 5 E1A gene (pAd.E1A-neo) into OVCAR-3 cells with 3β[N—(N′,N′-dimethylaminoethane)-carbamoyl] cholesterol (DC-Chol), a cationic derivative of cholesterol, and dioleoylphosphatidylethanol-amine (DOPE) (Ueno et al., 2000). Four OVCAR-E1A clones (OVCAR-E27, -E28, -E39, and -E43) were established. The -E27 and -E28 clones had similarly high expression of E1A relative to that of the -E39 and -E43 clones.

Other E1A transfections involved use of two adenoviral vectors: Ad.E1A(+), an adenovirus type 5-based vector containing E1A but lacking E1B and E3; and Ad.E1A(−), a control vector similar to Ad.E1A(+) but lacking E1A (Zhang et al., 1995;Yan et al., 1991). Viability of the transfected OVCAR-3 cells was verified by trypan blue exclusion. Briefly, 3×10⁵ cells were plated on 35-mm tissue culture dishes and 24 h later infected with 10 MOI Ad.E1A(−) or Ad.E1A(+) in replicates of three. Cells were stained with 0.2% trypan blue at 24, 48, 96, or 120 h after infection, and living cells and dead cells were counted under a microscope.

The PEA15 transfectants were established as follows. Two human ovarian cancer cell lines (SKOV3.ip1, OVCAR-3) and two breast cancer cell lines (MDA-MB-231, MDA-MB-435) were seeded on 100-mm plates and transfected with 10 μg of either a vector containing hemagglutinin (HA)-tagged PEA15 (Formstecher et al., 2001) or a control vector (pcDNA3) (Stratagene, La Jolla, Calif.) by means of DC-Chol cationic liposomes. Both plasmids contain a neomycin-resistance gene. At 48 h after transfection, cells were trypsinized, diluted 1:5 or 1:10, plated on 100-mm plates, and grown in RPMI 1640 containing 200 μg/ml of G418 (OVCAR-3) or DMEM/F12 containing 1,000 μg/ml of G418 (SKOV3.ip1). Three weeks later, colonies were counted (after staining with 0.5% crystal violet in 20% ethanol) and individual neomycin-resistant colonies were cloned and expanded to mass culture. Three SKOV3.ip1-PEA 15 clones (ip1-P1, -P2, -P3) were established and used for the experiments.

IX. Western Blot Analysis

Cells were washed three times with PBS and then lysed in lysis buffer (20 mM Na₂PO₄, [pH 7.4], 150 mM NaCl, 1% Triton X-100, 1% aprotinin, 1 mM phenylmethysulfonyl fluoride, 100 mM NaF, and 2 mM Na₃VO₄) as described previously (Ueno et al., 2000). PEA15 was extracted with NP-40 lysis buffer as described previously (Hao et al., 2001). The rabbit anti-PEA15 polyclonal antibody 4513 (used in a 1:1,000 dilution) was provided by Mark H. Ginsberg. Other primary antibodies used were ERK (in a 1:500 dilution) and phosphoERK (in a 1:500 dilution) (both from Santa Cruz Biotechnology, Santa Cruz, Calif.), actin (1:5,000) (Sigma-Aldrich Chemical Co, Saint Louis, Mo.), poly (ADP-ribose) polymerase (PARP) (1:1,000) (BD Biosciences), α-tubulin (1:5,000) (Sigma-Aldrich), and E1A (1:500) (BD Biosciences). The rabbit (1:8,000) and mouse (1:5,000) secondary antibodies were from Jackson ImmunoResearch (West Grove, Pa.).

X. Immunofluorescence Studies

Cells were grown for 1 day on Labtek chamber slides at 20,000 cells/well, after which medium was removed and the cells were washed three times with ice-cold PBS (5 min per wash) and fixed in 4% paraformaldehyde for 20 min at room temperature. After another three 5-min washes in ice-cold PBS, cells were permeabilized in 0.2% Triton-X in PBS for 10 min at room temperature, washed again in PBS, and blocked in 5% normal goat serum (DakoCytomation, Carpinteria, Calif.) for 1 h at room temperature. Cells were then incubated overnight at room temperature with the following primary antibodies: polyclonal rabbit anti-PEA15 antibody at a 1:100 dilution (SynPep, Dublin, Calif.) and monoclonal anti-mouse phosphoERK (Santa Cruz Biotechnology) at 1:50. The next day, the cells were washed three times with PBS and incubated for 1 h with either goat anti-rabbit FITC secondary antibody (Biosource, Camarillo, Calif.) at a 1:500 dilution or goat anti-mouse FITC secondary antibody (Molecular Probes, Eugene, Oreg.) at 1:500. Cells were visualized with either a confocal laser scanning microscope (Olympus, Japan) or a Zeiss AxioPlan2 fluorescence microscope. The nuclei were stained with propidium iodide (confocal) and 4′,6-diamidino-2-phenylindole (DAPI) (fluorescence) before mounting.

XI. Evaluation of Anchorage-Independent Growth

To assess anchorage-independent growth, an indicator of in vivo tumorigenicity (Zhang et al., 1995), the present inventors mixed cells at 37° C. with 0.5% agarose in complete medium and poured the mixture over a layer of 1% agarose in complete medium in six-well plates. The top layer was allowed to gel at 4° C. for 15 min, and the plates were then incubated at 37° C. for 3 weeks. At 3 weeks, the plates were stained by adding p-iodonitrotetrazolium violet to each well and incubated at 37° C. for 24 hours, after which the colonies were photographed with a Zeiss microscope and counted by using the software associated with the microscope.

XII. Evaluation of DNA Synthesis

DNA synthesis was measured with a BrdU labeling and detection kit (Roche Diagnostics, Indianapolis, Ind.) as follows. The parental cell line (OVCAR-3), the neomycin-pool control cells (OVCAR-NP), and two E1A transfectant clones (OVCAR-E27 and OVCAR-E28) were plated on a four-chamber slide in duplicate at 20,000 cell/well and incubated with BrdU labeling medium for 40 min at 37° C. Percentages of BrdU-labeled cells were evaluated by fluorescence microscopy with a Zeiss microscope.

In another set of experiments, cells were transfected with small interfering (si) RNA constructs against PEA15 or a scrambled control sequence (described below) for 30 h, after which culture medium containing BrdU was added and the cells were incubated for an additional 20 h. Direct immunofluorescence staining was performed according to the manufacturer's protocol (Becton Dickinson, San Jose, Calif.) and the samples were analyzed on a BD FACScan flow cytometer.

XIII. siRNA Transfection

Cells (2×10⁵ cells per well) were seeded in six-well culture plates in RPMI medium supplemented with 10% FBS at 30% to 50% confluence. The next day, cells were transfected with the siRNA (GGAAGACATCCCCAGCGAATT; SEQ ID NO:31) or a scrambled siRNA duplex (siControl) (Dharmacon Inc., Lafayette, Colo.) at a final siRNA concentration of 200 nM by using oligofectamine (Invitrogen, Palo Alto, Calif.) and incubated for 4 hours. Next, FBS medium was added to reach a final concentration of 20% FBS in the wells. Cells were fixed 24, 48, or 96 h later and subjected to immunofluorescence studies.

XIV. Nuclear/Cytoplasmic Fractionation

OVCAR-NP and OVCAR-E27 cells were plated at 2×10⁵ cells/well in RPMI medium containing 10% FBS and incubated at 37° C. for 24 h, after which they were grown in medium containing 2% serum overnight and then stimulated with 20% serum for 30 min. Cells were pelleted and the nuclear and cytoplasmic fractions were separated with a nuclear/cytosol fractionation kit according to the manufacturer's protocol (BioVision, Mountain View, Calif.). Protein concentrations in the cytoplasmic and nuclear fractions were measured with a protein assay kit from Bio-Rad Laboratories (Hercules, Calif.).

XV. Elk-1 Transcription Assay

The PathDetect Elk-1 trans-reporting system (Stratagene, La Jolla, Calif.) was used according to the manufacturer's protocol. Briefly, OVCAR-3-NP and OVCAR-3-E1A cells (2×10⁵ cells/well) were plated on 35-mm plates, and the next day the cells were cotransfected with pFA2-Elk-1, pFR-luciferase, and the internal transfection control vector pRL-TK (Promega Biosciences, San Luis Obispo, Calif.). Cells were grown in medium containing 0.5% serum overnight and then stimulated with medium containing 20% serum for 30 min. Cells were then harvested and Elk-1 transcription was measured in terms of luciferase activity by using Promega's dual luciferase assay system according to the manufacturer's protocol. Elk-1-luciferase activity was normalized against the internal control luciferase activity of pRL-TK.

In another set of experiments, OVCAR-E1A cells (2×10⁵ cells/well) were transfected with siControl or siPEA15 constructs as described under “siRNA Transfection”. Twenty-four hours later, cells were cotransfected with pFA2-Elk-1, pFR-luciferase, and pRL-TK (Promega Biosciences), and after another 24 h, cells were grown in medium containing 0.5% serum overnight and then stimulated with medium containing 20% serum for 30 min. Cells were then harvested and Elk-1 transcription was measured as described above.

### Example 16

### Alternative Embodiments

In a particular embodiment of the present invention, other molecules involved in the anti-tumor activity of E1A gene therapy are identified, such as by protein profiling and/or gene microarray. Another modification of the invention could regard the inhibition of expression of PEA15 by PEA15-siRNA. In the event, for example, that the PEA15-siRNA may not completely inhibit expression of PEA15 protein, perhaps because the transfection efficiency may be too low in a particular cell line or the chosen target sequence may not be the optimal sequence. To address these situations, other transfection reagents may be utilized and/or other target sequences in PEA 15 are used for synthesis of a new PEA 15-siRNA from another region of the gene.

An additional alternative embodiment is directed to the delivery system. If an alternative system from a non-viral delivery system described herein is desired, such as because of sub-optimal gene-delivery efficiency, a replication-deficient adenovirus gene delivery system may be used. This should be able to transfer PEA15 directly to tumor cells in vivo. The advantage of such a delivery system is that high titers of the virus can be easily purified and the infection efficiency is high.

Finally, an additional potential mechanism by which tumorigenicity is suppressed is through induction of apoptosis. Although PEA15 inhibits FAS and TNFR1-mediated apoptosis in MCF-7 cells overexpressing PEA15 (Condorelli et al., 1999), PEA15 is not known to inhibit TNF-induced apoptosis in NIH3T3 cells. Further, when either or both phosphorylation sites of PEA15 (Ser104 or Ser116) are phosphorylated, PEA15 did not protect cells from Fas-induced apoptosis (Estelles et al., 1999). Regardless of results (reduction of cell proliferation, induction of apoptosis), gene microarray studies described herein will provide a powerful tool to reveal the molecular mechanism related to anti-tumor activity of PEA15.

### REFERENCES

All patents and publications mentioned in the specification are indicative of the levels of those skilled in the art to which the invention pertains. All patents and publications are herein incorporated by reference in their entirety to the same extent as if each individual publication was specifically and individually indicated to be incorporated by reference.

PATENTS AND PATENT APPLICATIONS

U.S. Pat. No. 4,554,101
U.S. 2003/0049702
U.S. 2003/0215835
U.S. 2003/0134283
WO200240680
WO2003074007
WO2004108961

### PUBLICATIONS

Araujo H, Danziger N, Cordier J, Glowinski J, and Chneiweiss H Characterization of PEA-15, a major substrate for protein kinase C in astrocytes. J Biol Chem, 1993; 268:5911-20.
Araujo, H., Danziger, N., Cordier, J., Glowinski, J., and Chneiweiss, H. Characterization of PEA-15, a major substrate for protein kinase C in astrocytes. J Biol Chem, 268: 5911-5920., 1993.
Bargmann, C. I., Hung, M. C., and Weinberg, R. A. Multiple independent activations of the neu oncogene by a point mutation altering the transmembrane domain of p185. Cell, 45: 649-657, 1986.
Barker S D, Coolidge C J, Kanerva A, Hakkarainen T, Yamamoto M, Liu B, Rivera A A, Bhoola S M, Barnes M N, Alvarez R D, Curiel D T, Hemminki A. The secretory leukoprotease inhibitor (SLPI) promoter for ovarian cancer gene therapy. J Gene Med. April; 5(4):300-10, 2003.
Barker S D, Dmitriev I P, Nettelbeck D M, Liu B, Rivera A A, Alvarez R D, Curiel D T, Hemminki A. Combined transcriptional and transductional targeting improves the specificity and efficacy of adenoviral gene delivery to ovarian carcinoma. Gene Ther. 2003 July; 10(14):1198-204.
Belotti, D., Vergani, V., Drudis, T., Borsotti, P., Pitelli, M. R., Viale, G., Giavazzi, R., and Taraboletti, G. The microtubule-affecting drug paclitaxel has antiangiogenic activity. Clin Cancer Res, 2: 1843-1849, 1996.
Bera T K, Guzman R C, Miyamoto S, et al. Identification of a mammary transforming gene (MAT1) associated with mouse mammary carcinogenesis. Proc Natl Acad Sci USA, 1994; 91:9789-93.
Bernstein, E., Denli, A. M., and Hannon, G. J. The rest is silence. Rna, 7: 1509-1521, 2001.
Blagosklonny, M. V., Schulte, T., Nguyen, P., Trepel, J., and Neckers, L. M. Taxol-induced apoptosis and phosphorylation of Bcl-2 protein involves c-Raf-1 and represents a novel c-Raf-1 signal transduction pathway. Cancer Res, 56: 1851-1854, 1996.
Burack, W. R. and Shaw, A. S. Signal transduction: hanging on a scaffold. Curr Opin Cell Biol, 12: 211-216, 2000.
Burkhart, C. A., Berman, J. W., Swindell, C. S., and Horwitz, S. B. Relationship between the structure of taxol and other taxanes on induction of tumor necrosis factor-alpha gene expression and cytotoxicity. Cancer Res, 54: 5779-5782, 1994.
Byrd P J, Grand R J, and Gallimore P H Differential transformation of primary human embryo retinal cells by adenovirus E1 regions and combinations of E1A+ras. Oncogene, 1988; 2:477-84.
Chang J Y, Xia W, Shao R, et al. The tumor suppression activity of E1A in HER-2/neu-overexpressing breast cancer. Oncogene, 1997; 14:561-8.
Chang, J. Y., Xia, W., Shao, R., Sorgi, F., Hortobagyi, G. N., Huang, L., and Hung, M. C. The tumor suppression activity of E1A in HER-2/neu-overexpressing breast cancer. Oncogene, 14: 561-568, 1997.
Cobb, M. H. MAP kinase pathways. Prog Biophys Mol Biol, 71: 479-500, 1999.
Condorelli G, Trencia A, Vigliotta G, et al. Multiple members of the mitogen-activated protein kinase family are necessary for PED/PEA-15 anti-apoptotic function. J Biol Chem, 2002; 277:11013-8.
Condorelli G, Vigliotta G, Cafieri A, et al. PED/PEA-15: an anti-apoptotic molecule that regulates FAS/TNFR1-induced apoptosis. Oncogene, 1999; 18:4409-15.
Condorelli G, Vigliotta G, Iavarone C, et al. PED/PEA-15 gene controls glucose transport and is overexpressed in type 2 diabetes mellitus. Embo J, 1998; 17:3858-66.
Condorelli, G., Trencia, A., Vigliotta, G., Perfetti, A., Goglia, U., Cassese, A., Musti, A. M., Miele, C., Santopietro, S., Formisano, P., and Beguinot, F. Multiple members of the mitogen-activated protein kinase family are necessary for PED/PEA-15 anti-apoptotic function. J Biol Chem, 277: 11013-11018, 2002.
Condorelli, G., Vigliotta, G., Cafieri, A., Trencia, A., Andalo, P., Oriente, F., Miele, C., Caruso, M., Formisano, P., and Beguinot, F. PED/PEA-15: an anti-apoptotic molecule that regulates FAS/TNFR1-induced apoptosis. Oncogene, 18: 4409-4415., 1999.
Condorelli, G., Vigliotta, G., Iavarone, C., Caruso, M., Tocchetti, C. G., Andreozzi, F., Cafieri, A., Tecce, M. F., Formisano, P., Beguinot, L., and Beguinot, F. PED/PEA-15 gene controls glucose transport and is overexpressed in type 2 diabetes mellitus. Embo J, 17: 3858-3866., 1998.
Cook, J. L., May, D. L., Wilson, B. A., Holskin, B., Chen, M. J., Shalloway, D., and Walker, T. A. Role of tumor necrosis factor-alpha in E1A oncogene-induced susceptibility of neoplastic cells to lysis by natural killer cells and activated macrophages. J Immunol, 142: 4527-4534, 1989.
Coussens, L., Yang, F. T., Liao, Y. C., Chen, E., Gray, A., McGrath, J., Seeburg, P. H., Libermann, T. A., Schlessinger, J., Francke, U., Levinson, A., and Ullrich, A. Tyrosine kinase receptor with extensive homology to EGF receptor shares chromosomal location with neu oncogene. Science, 230: 1132-1139, 1985.
Danziger N, Yokoyama M, Jay T, et al. Cellular expression, developmental regulation, and phylogenic conservation of PEA-15, the astrocytic major phosphoprotein and protein kinase C substrate. J Neurochem, 1995; 64:1016-25.
Danziger, N., Yokoyama, M., Jay, T., Cordier, J., Glowinski, J., and Chneiweiss, H. Cellular expression, developmental regulation, and phylogenic conservation of PEA-15, the astrocytic major phosphoprotein and protein kinase C substrate. J Neurochem, 64: 1016-1025., 1995.
Debbas M and White E Wild-type p53 mediates apoptosis by E1A, which is inhibited by E1B. Genes Dev, 1993; 7:546-54.
Debbas, M. and White, E. Wild-type p53 mediates apoptosis by E1A, which is inhibited by E1B. Genes Dev, 7: 546-554, 1993.
Deng J, Kloosterbooer F, Xia W, Hung M C. The NH(2)-terminal and conserved region 2 domains of adenovirus E1A mediate two distinct mechanisms of tumor suppression. Cancer Res. January 15; 62(2):346-50, 2002.
Deng J, Xia W, and Hung M C Adenovirus 5 E1A-mediated tumor suppression associated with E1A-mediated apoptosis in vivo. Oncogene, 1998; 17:2167-75.
Douglas J L, Gopalakrishnan S, and Quinlan M P Modulation of transformation of primary epithelial cells by the second exon of the Ad5 E1A12S gene. Oncogene, 1991; 6:2093-103.
Downward, J. Targeting RAS signalling pathways in cancer therapy. Nat Rev Cancer, 3: 11-22, 2003.
Dunton, C. New options for the treatment of advanced ovarian cancer. Semin Oncol, 24(supp1.5): 2-11, 1997.
Elbashir, S. M., Harborth, J., Lendeckel, W., Yalcin, A., Weber, K., and Tuschl, T. Duplexes of 21-nucleotide RNAs mediate RNA interference in cultured mammalian cells. Nature, 411: 494-498, 2001.
Elbashir, S. M., Lendeckel, W., and Tuschl, T. RNA interference is mediated by 21- and 22-nucleotide RNAs. Genes Dev, 15: 188-200, 2001.
Elbashir, S. M., Martinez, J., Patkaniowska, A., Lendeckel, W., and Tuschl, T. Functional anatomy of siRNAs for mediating efficient RNAi in Drosophila melanogaster embryo lysate. Embo J, 20: 6877-6888, 2001.
Estelles A, Charlton C A, and Blau H M The phosphoprotein protein PEA-15 inhibits Fas—but increases TNF-R1-mediated caspase-8 activity and apoptosis. Dev Biol, 1999; 216:16-28.
Estelles A, Yokoyama M, Nothias F, et al. The major astrocytic phosphoprotein PEA-15 is encoded by two mRNAs conserved on their full length in mouse and human. J Biol Chem, 1996; 271:14800-6.
Estelles, A., Charlton, C. A., and Blau, H. M. The phosphoprotein protein PEA-15 inhibits Fas-but increases TNF-R1-mediated caspase-8 activity and apoptosis. Dev Biol, 216: 16-28., 1999.
Formstecher E, Ramos J W, Fauquet M, et al. PEA-15 mediates cytoplasmic sequestration of ERK MAP kinase. Dev Cell, 2001; 1:239-50.
Formstecher, E., Ramos, J. W., Fauquet, M., Calderwood, D. A., Hsieh, J. C., Canton, B., Nguyen, X. T., Barnier, J. V., Camonis, J., Ginsberg, M. H., and Chneiweiss, H. PEA-15 mediates cytoplasmic sequestration of ERK MAP kinase. Dev Cell, 1: 239-250., 2001.
Frisch S M and Dolter K E Adenovirus E1a-mediated tumor suppression by a c-erbB-2/neu-independent mechanism. Cancer Res, 1995; 55:5551-5.
Frisch S M and Mymryk J S Adenovirus-5 E1A: paradox and paradigm. Nat Rev Mol Cell Biol, 2002; 3:441-52.
Frisch S M Antioncogenic effect of adenovirus E1A in human tumor cells. Proc Natl Acad Sci U S A, 1991; 88:9077-81.
Frisch S M E1A induces the expression of epithelial characteristics. J Cell Biol, 1994; 127:1085-96.
Frisch S M, Reich R, Collier I E, et al. Adenovirus E1A represses protease gene expression and inhibits metastasis of human tumor cells. Oncogene, 1990; 5:75-83.
Frisch, S. M. E1A induces the expression of epithelial characteristics. J Cell Biol, 127: 1085-1096, 1994.
Frisch, S. M. E1A-oncogene or tumor suppressor? Bioessays, 17, 1995.
Frisch, S. M., Reich, R., Collier, I. E., Genrich, L. T., Martin, G., and Goldberg, G. I. Adenovirus E1A represses protease gene expression and inhibits metastasis of human tumor cells. Oncogene, 5: 75-83, 1990.
Fukuda, M., Gotoh, Y., and Nishida, E. Interaction of MAP kinase with MAP kinase kinase: its possible role in the control of nucleocytoplasmic transport of MAP kinase. Embo J, 16: 1901-1908, 1997.
Gaumont-Leclerc M F, Mukhopadhyay U K, Goumard S, and Ferbeyre G PEA-15 is inhibited by adenovirus E1A and plays a role in ERK nuclear export and Ras-induced senescence. J Biol Chem, 2004;
Gille, H., Kortenjann, M., Thomae, O., Moomaw, C., Slaughter, C., Cobb, M. H., and Shaw, P. E. ERK phosphorylation potentiates Elk-1-mediated ternary complex formation and transactivation. Embo J, 14: 951-962, 1995.
Gupta, K., Kshirsagar, S., Li, W., Gui, L., Ramakrishnan, S., Gupta, P., Law, P. Y., and Hebbel, R. P. VEGF prevents apoptosis of human microvascular endothelial cells via opposing effects on MAPK/ERK and SAPK/JNK signaling. Experimental cell research., 247(2): 495-504, 1999.
Hao C, Beguinot F, Condorelli G, et al. Induction and intracellular regulation of tumor necrosis factor-related apoptosis-inducing ligand (TRAIL) mediated apotosis in human malignant glioma cells. Cancer Res, 2001; 61:1162-70.
Hill, J. M., Vaidyanathan, H., Ramos, J. W., Ginsberg, M. H., and Werner, M. H. Recognition of ERK MAP kinase by PEA-15 reveals a common docking site within the death domain and death effector domain. Embo J, 21: 6494-6504, 2002.
Hortobagyi G N, Ueno N T, Xia W Y, et al. Cationic liposome-mediated E1A gene transfer to human breast and ovarian cancer cells and its biologic effects: A phase I clinical trial. Journal of Clinical Oncology, 2001; 19:3422-33.
Hortobagyi, G. N., Ueno, N. T., Xia, W. Y., Zhang, S., Wolf, J. K., Putnam, J. B., Weiden, P. L., Willey, J. S., Carey, M., Branham, D. L., Payne, J. Y., Tucker, S. D., Bartholomeusz, C., Kilbourn, R. G., De Jager, R. L., Sneige, N., Katz, R. L., Anklesaria, P., Ibrahim, N. K., Murray, J. L., Theriault, R. L., Valero, V., Gershenson, D. M., Bevers, M. W., Huang, L., and et al. Cationic liposome-mediated E1A gene transfer to human breast and ovarian cancer cells and its biologic effects: A phase I clinical trial. Journal of Clinical Oncology, 19: 3422-3433, 2001.
Horwitz, S. B. Taxol (paclitaxel): mechanisms of action. Ann Oncol, 5 suppl 6: S3-6, 1994.
Houweling A, van den Elsen P J, and van der Eb A J Partial transformation of primary rat cells by the leftmost 4.5% fragment of adenovirus 5 DNA. Virology, 1980; 105:537-50.
Hwang S, Kuo W L, Cochran J F, et al. Assignment of HMAT1, the human homolog of the murine mammary transforming gene (MAT1) associated with tumorigenesis, to 1q21.1, a region frequently gained in human breast cancers. Genomics, 1997; 42:540-2.
Jemal A, Tiwari R C, Murray T, et al. Cancer statistics, 2004. CA Cancer J Clin, 2004; 54:8-29.
Jemal, A., Murray, T., Samuels, A., Ghafoor, A., Ward, E., and Thun, M. J. Cancer statistics, 2003. CA: a cancer journal for clinicians., 53: 5-26, 2003.
Kitsberg, D., Formstecher, E., Fauquet, M., Kubes, M., Cordier, J., Canton, B., Pan, G., Rolli, M., Glowinski, J., and Chneiweiss, H. Knock-out of the neural death effector domain protein PEA-15 demonstrates that its expression protects astrocytes from TNFalpha-induced apoptosis. J Neurosci, 19: 8244-8251., 1999.
Klauber, N., Parangi, S., Flynn, E., Hamel, E., and D'Amato, R. J. Inhibition of angiogenesis and breast cancer in mice by the microtubule inhibitors 2-methoxyestradiol and taxol. Cancer Res, 57: 81-86, 1997.
Kubes M, Cordier J, Glowinski J, Girault J A, and Chneiweiss H Endothelin induces a calcium-dependent phosphorylation of PEA-15 in intact astrocytes: identification of Ser104 and Ser116 phosphorylated, respectively, by protein kinase C and calcium/calmodulin kinase II in vitro. J Neurochem, 1998; 71:1307-14.
Kubes, M., Cordier, J., Glowinski, J., Girault, J. A., and Chneiweiss, H. Endothelin induces a calcium-dependent phosphorylation of PEA-15 in intact astrocytes: identification of Ser104 and Ser116 phosphorylated, respectively, by protein kinase C and calcium/calmodulin kinase II in vitro. J Neurochem, 71: 1307-1314., 1998.
Land H, Parada L F, and Weinberg R A Tumorigenic conversion of primary embryo fibroblasts requires at least two cooperating oncogenes. Nature, 1983; 304:596-602.
Lee W P, Liao Y, Robinson D, et al. Ax1-Gas6 interaction counteracts E1A-mediated cell growth suppression and proapoptotic activity. Mol Cell Biol, 1999; 19:8075-82.
Lewis, T. S., Shapiro, P. S., and Ahn, N. G. Signal transduction through MAP kinase cascades. Adv Cancer Res, 74: 49-139, 1998.
Li, S, and Huang, L. In vivo gene transfer via intravenous administration of cationic lipid-protamine-DNA (LPD) complexes. Gene Ther, 4: 891-900, 1997.
Li, S., Rizzo, M. A., Bhattacharya, S., and Huang, L. Characterization of cationic lipid-protamine-DNA (LPD) complexes for intravenous gene delivery. Gene Ther, 5: 930-937, 1998.
Li, S., Wu, S. P., Whitmore, M., Loeffert, E. J., Wang, L., Watkins, S. C., Pitt, B. R., and Huang, L. Effect of immune response on gene transfer to the lung via systemic administration of cationic lipidic vectors. Am J Physiol, 276: L796-804, 1999.
Lieu, C. H., Liu, C. C., Yu, T. H., Chen, K. D., Chang, Y. N., and Lai, Y. K. Role of mitogen-activated protein kinase in taxol-induced apoptosis in human leukemic U937 cells. Cell Growth & Differentiation, 9: 767-776, 1998.
Ling, Y. H., Tornos, C., and Perez-Soler, R. Phosphorylation of Bcl-2 is a marker of M phase events and not a determinant of apoptosis. The Journal of biological chemistry, 273(30): 18984-18991, 1998.
Lowe, S. W. and Ruley, H. E. Stabilization of the p53 tumor suppressor is induced by adenovirus 5 E1A and accompanies apoptosis. Genes Dev, 7: 535-545, 1993.
Lowe, S. W., Ruley, H. E., Jacks, T., and Housman, D. E. p53-dependent apoptosis modulates the cytotoxicity of anticancer agents. Cell, 74: 957-967, 1993.
MacKeigan, J. P., Collins, T. S., and Ting, J. P. MEK inhibition enhances paclitaxel-induced tumor apoptosis. J Biol Chem, 275: 38953-38956, 2000.
MacKeigan, J. P., Collins, T. S., Ting, J. P., Lineberger Comprehensive Cancer Center, D. o. M., and Immunology, D. o. S. U. o. N.C. C. H. N.C. U.S.A. MEK inhibition enhances paclitaxel-induced tumor apoptosis. The Journal of biological chemistry., 275(50): 38953-38956, 2000.
Malumbres, M. and Barbacid, M. To cycle or not to cycle: a critical decision in cancer. Nat Rev Cancer, 1: 222-231, 2001.
Marcellus, R. C., Teodoro, J. G., Wu, T., Brough, D. E., Ketner, G., Shore, G. C., and Branton, P. E. Adenovirus type 5 early region 4 is responsible for E1A-induced p53-independent apoptosis. J Virol, 70: 6207-6215, 1996.
McDaid, H. M. and Horwitz, S. B. Selective potentiation of paclitaxel (taxol)-induced cell death by mitogen-activated protein kinase kinase inhibition in human cancer cell lines. Mol Pharmacol, 60: 290-301, 2001.
McDaid, H. M., Horwitz, S. B., and Department of Molecular Pharmacology, A. E. C. o. M. B. N.Y. U.S.A. Selective potentiation of paclitaxel (taxol)-induced cell death by mitogen-activated protein kinase kinase inhibition in human cancer cell lines. Molecular pharmacology., 60(2): 290-301, 2001.
Mymryk J S Tumour suppressive properties of the adenovirus 5 E1A oncogene. Oncogene, 1996; 13:1581-9.
Mymryk, J. S. Tumor suppressive properties of the adenovirus 5 E1A oncogene. Oncogene, 13: 1581-1589, 1996.
Mymryk, J. S., Shire, K., and Bayley, S. T. Induction of apoptosis by adenovirus type 5 E1A in rat cells requires a proliferation block. Oncogene, 9: 1187-1193, 1994.
Nabel, E. G., Gordon, D., Yang, Z. Y., Xu, L., San, H., Plautz, G. E., Wu, B. Y., Gao, X., Huang, L., and Nabel, G. J. Gene transfer in vivo with DNA-liposome complexes: lack of autoimmunity and gonadal localization. Hum Gene Ther, 3: 649-656, 1992.
Nabel, G. J., Nabel, E. G., Yang, Z. Y., Fox, B. A., Plautz, G. E., Gao, X., Huang, L., Shu, S., Gordon, D., and Chang, A. E. Direct gene transfer with DNA-liposome complexes in melanoma: expression, biologic activity, and lack of toxicity in humans. Proc Natl Acad Sci USA, 90: 11307-11311, 1993.
Nelson C C, Braithwaite A W, Silvestro M, and Bellett A J E1A-dependent expression of adenovirus genes in OTF963 embryonal carcinoma cells: role of E1a-induced differentiation. Proc Natl Acad Sci USA, 1990; 87:8041-5.
Nelson, C. C., Braithwaite, A. W., Silvestro, M., and Bellett, A. J. E1A-dependent expression of adenovirus genes in OTF963 embryonal carcinoma cells: role of E1a-induced differentiation. Proc Natl Acad Sci USA, 87: 8041-8045, 1990.
Nevins, J. R. Adenovirus E1A-dependent trans-activation of transcription. Semin Cancer Biol, l: 59-68, 1990.
Nevins, J. R., Raychaudhuri, P., Yee, A. S., Rooney, R. J., Kovesdi, I., and Reichel, R. Transactivation by the adenovirus E1A gene. Biochem Cell Biol, 66: 578-583, 1988.
Nikitin, A. Y., Juarez-Perez, M. I., Li, S., Huang, L., and Lee, W. H. RB-mediated suppression of spontaneous multiple neuroendocrine neoplasia and lung metastases in Rb+/−mice. Proc Natl Acad Sci USA, 96: 3916-3921, 1999.
Patterson A, Harris A L. Molecular chemotherapy for breast cancer. Drugs Aging. 1999 February; 14(2):75-90.
Ramos J W, Hughes P E, Renshaw M W, et al. Death effector domain protein PEA-15 potentiates Ras activation of extracellular signal receptor-activated kinase by an adhesion-independent mechanism. Mol Biol Cell, 2000; 11:2863-72.
Rao L, Debbas M, Sabbatini P, et al. The adenovirus E1A proteins induce apoptosis, which is inhibited by the E1B 19-kDa and Bcl-2 proteins. Proc Natl Acad Sci USA, 1992; 89:7742-6.
Rao, L., Debbas, M., Sabbatini, P., Hockenbery, D., Korsmeyer, S., and White, E. The adenovirus E1A proteins induce apoptosis, which is inhibited by the E1B 19-kDa and Bcl-2 proteins. Proc Natl Acad Sci USA, 89: 7742-7746, 1992.
Renault F, Formstecher E, Callebaut I, Junier M P, and Chneiweiss H The multifunctional protein PEA-15 is involved in the control of apoptosis and cell cycle in astrocytes. Biochem Pharmacol, 2003; 66:1581-8.
Rodrigues, M., Dion, P., Sircar, S., and Weber, J. M. Tumor necrosis factor mediated cytolysis requires the adenovirus E1a protein but not the transformed phenotype. Virus Res, 15: 231-236, 1990.
Ruley H E Adenovirus early region 1 A enables viral and cellular transforming genes to transform primary cells in culture. Nature, 1983; 304:602-6.
Sabbatini, P., Lin, J., Levine, A. J., and White, E. Essential role for p53-mediated transcription in E1A-induced apoptosis. Genes Dev, 9: 2184-2192, 1995.
Sanchez-Prieto, R., Quintanilla, M., Cano, A., Lleonart, M., Martin, P., Anaya, A., and Cajal, S. Carcinoma cell lines become sensitive to DNA-damaging agents by the expression of the adenovirus E1A gene. Oncogene, 13: 1083-1092, 1996.
Scatena, C. D., Stewart, Z. A., Mays, D., Tang, L. J., Keefer, C. J., Leach, S. D., and Pietenpol, J. A. Mitotic phosphorylation of Bcl-2 during normal cell cycle progression and Taxol-induced growth arrest. The Journal of biological chemistry, 273(46): 30777-30784, 1998.
Schaeffer, H. J. and Weber, M. J. Mitogen-activated protein kinases: specific messages from ubiquitous messengers. Mol Cell Biol, 19: 2435-2444, 1999.
Schaeffer, H. J., Catling, A. D., Eblen, S. T., Collier, L. S., Krauss, A., and Weber, M. J. MP1: a MEK binding partner that enhances enzymatic activation of the MAP kinase cascade. Science, 281: 1668-1671, 1998.
Schechter, A. L., Hung, M. C., Vaidyanathan, L., Weinberg, R. A., Yang, F. T., Francke, U., Ullrich, A., and Coussens, L. The neu gene: an erbB-homologous gene distinct from and unlinked to the gene encoding the EGF receptor. Science, 229: 976-978, 1985.
Schiff, P. B. and Horwitz, S. B. Taxol stabilizes microtubules in mouse fibroblast cells. Proc Natl Acad Sci USA, 77: 1561-1565, 1980.
Shao R, Karunagaran D, Zhou B P, et al. Inhibition of nuclear factor-kappaB activity is involved in E1A-mediated sensitization of radiation-induced apoptosis. J Biol Chem, 1997; 272:32739-42.
Shao R, Xia W, and Hung M C Inhibition of angiogenesis and induction of apoptosis are involved in E1A-mediated bystander effect and tumor suppression. Cancer Res, 2000; 60:3123-6.
Shao, R., Karunagaran, D., Zhou, B. P., Li, K., Lo, S. S., Deng, J., Chiao, P., and Hung, M. C. Inhibition of nuclear factor-kappaB activity is involved in E1A-mediated sensitization of radiation-induced apoptosis. J Biol Chem, 272: 32739-32742, 1997.
Shisler, J., Duerksen, H. P., Hermiston, T. M., Wold, W. S., and Gooding, L. R. Induction of susceptibility to tumor necrosis factor by E1A is dependent on binding to either p300 or p105-Rb and induction of DNA synthesis. J Virol, 70: 68-77, 1996.
Shtil, A. A., Mandlekar, S., Yu, R., Walter, R. J., Hagen, K., Tan, T. H., Roninson, I. B., and Kong, A. N. Differential regulation of mitogen-activated protein kinases by microtubule-binding agents in human breast cancer cells. Oncogene., 18(2): 377-384, 1999.
Tan, Y., Li, S., Pitt, B. R., and Huang, L. The inhibitory role of CpG immunostimulatory motifs in cationic lipid vector-mediated transgene expression in vivo. Hum Gene Ther, 10: 2153-2161, 1999.
Tang, C., Willingham, M. C., Reed, J. C., Miyashita, T., Ray, S., Ponnathpur, V., Huang, Y., Mahoney, M. E., Bullock, G., and Bhalla, K. High levels of p26BCL-2 oncoprotein retard taxol-induced apoptosis in human pre-B leukemia cells. Leukemia, 8: 1960-1969, 1994.
Teodoro J G, Shore G C, and Branton P E Adenovirus E1A proteins induce apoptosis by both p53-dependent and p53-independent mechanisms. Oncogene, 1995; 11:467-74.
Teodoro, J. G., Shore, G. C., and Branton, P. E. Adenovirus E1A proteins induce apoptosis by both p53-dependent and p53-independent mechanisms. Oncogene, 11: 467-474, 1995.
Trencia A, Perfetti A, Cassese A, et al. Protein kinase B/Akt binds and phosphorylates PED/PEA-15, stabilizing its antiapoptotic action. Mol Cell Biol, 2003; 23:4511-21.
Tsang W S, Quackenbush L S, Chow B K, Tiu S H, He J G, Chan S M. Organization of the shrimp vitellogenin gene: evidence of multiple genes and tissue specific expression by the ovary and hepatopancreas. Gene, January 16; 303:99-109, 2003.
Ueno N T, Bartholomeusz C, Herrmann J L, et al. E1A-mediated paclitaxel sensitization in HER-2/neu-overexpressing ovarian cancer SKOV3.ip1 through apoptosis involving the caspase-3 pathway. Clin Cancer Res, 2000; 6:250-9.
Ueno N T, Yu D, and Hung M-C Chemosensitization of HER2/neu-overexpressing human breast cancer cells to paclitaxel(Taxol) by adenovirus type 5 E1A. Oncogene, 1997; 15:953-60.
Ueno, N. T., Bartholomeusz, C., Herrmann, J. L., Estrov, Z., Shao, R., Andreeff, M., Price, J., Paul, R. W., Anklesaria, P., Yu, D., and Hung, M. C. E1A-mediated paclitaxel sensitization in HER-2/neu-overexpressing ovarian cancer SKOV3.ip1 through apoptosis involving the caspase-3 pathway. Clin Cancer Res, 6: 250-259, 2000.
Ueno, N. T., Bartholomeusz, C., Xia, W., Anklesaria, P., Bruckheimer, E. M., Mebel, E., Paul, R., Li, S., Yo, G. H., Huang, L., and Hung, M. C. Systemic gene therapy in human xenograft tumor models by liposomal delivery of the E1A gene. Cancer Res, 62: 6712-6716, 2002.
Ueno, N. T., Hung, M. C., Zhang, S., Xia, W., Willey, J. S., Warner, D. L., Payne, J. Y., Nandagiri, R., Aldridge, R., De Jager, R. L., Tucker, S., Putnam, J. B., Sneige, N., Katz, R. L., Spohn, W. H., Bast, R. C., Holmes, F. A., Ibrahim, N. K., Theriault, R. L., Rahman, Z. U., Valero, V., Kudelka, A. P., Kavanagh, J. J., Freedman, R. S., Wolf, J. K., Gershenson, D. M., Bevers, M. W., Huang, L., Lopez-Bernstein, G., and Hortobagyi, G. N. Phase I E1A gene therapy in patients with advanced breast and ovarian cancers. In: Proc Annu Meet Am Assoc Cancer Res., New Orleans, 1998, pp. 360.
Ueno, N. T., Yu, D., and Hung, M. C. Chemosensitization of HER-2/neu-overexpressing human breast cancer cells to paclitaxel (Taxol) by adenovirus type 5 E1A. Oncogene, 15: 953-960, 1997.
van Groningen, J. J., Cornelissen, I. M., van Muijen, G. N., Bloemers, H. P., and Swart, G. W. Simultaneous suppression of progression marker genes in the highly malignant human melanoma cell line BLM after transfection with the adenovirus-5 E1A gene. Biochemical & Biophysical Research Communications, 225: 808-816, 1996.
Wang, T. H., Popp, D. M., Wang, H. S., Saitoh, M., Mural, J. G., Henley, D.C., Ichijo, H., and Wimalasena, J. Microtubule dysfunction induced by paclitaxel initiates apoptosis through both c-Jun N-terminal kinase (JNK)-dependent and -independent pathways in ovarian cancer cells. J Biol Chem, 274(12): 8208-8216, 1999.
Wang, T. H., Wang, H. S., Ichijo, H., Giannakakou, P., Foster, J. S., Fojo, T., and Wimalasena, J. Microtubule-interfering agents activate c-Jun N-terminal kinase/stress-activated protein kinase through both Ras and apoptosis signal-regulating kinase pathways. The Journal of biological chemistry., 273(9): 4928-4936, 1998.
Waskiewicz, A. J. and Cooper, J. A. Mitogen and stress response pathways: MAP kinase cascades and phosphatase regulation in mammals and yeast. Curr Opin Cell Biol, 7: 798-805, 1995.
White, E. Regulation of p53-dependent apoptosis by E1A and E1B. Curr Top Microbiol Immunol, 199: 34-58, 1995.
Whitehurst A W, Robinson F L, Moore M S, and Cobb M H The death effector domain protein PEA-15 prevents nuclear entry of ERK2 by inhibiting required interactions. J Biol Chem, 2004; 279: 12840-7.
Whitmarsh, A. J. and Davis, R. J. Structural organization of MAP-kinase signaling modules by scaffold proteins in yeast and mammals. Trends Biochem Sci, 23: 481-485, 1998.
Whitmore, M. M., Li, S., Falo, L., Jr., and Huang, L. Systemic administration of LPD prepared with CpG oligonucleotides inhibits the growth of established pulmonary metastases by stimulating innate and acquired antitumor immune responses. Cancer Immunology, Immunotherapy, 50: 503-514, 2001.
Whitmore, M., Li, S., and Huang, L. LPD lipopolyplex initiates a potent cytokine response and inhibits tumor growth. Gene Ther, 6: 1867-1875, 1999.
Wolf, J K, Jenkins, A D. Gene therapy for ovarian cancer (review). Int J. Oncol. September; 21(3):461-8, 2002.
Xia, Z., Dickens, M., Raingeaud, J., Davis, R. J., Greenberg, M. E., and Department of Neurology, C. S. H. H. M. S. B. M. A. U.S.A. Opposing effects of ERK and JNK-p38 MAP kinases on apoptosis. Science., 270(5240): 1326-1331, 1995.
Xing, X., Liu, V., Xia, W., Stephens, L. C., Huang, L., Lopez-Berestein, G., and Hung, M. C. Safety studies of the intraperitoneal injection of E1A-liposome complex in mice. Gene Ther, 4: 238-243, 1997.
Xing, X., Zhang, S., Chang, J. Y., Tucker, S. D., Chen, H., Huang, L., and Hung, M. C. Safety study and characterization of E1A-liposome complex gene delivery in an ovarian cancer model. Gene Ther, 5: 1538-1544, 1998.
Xu F, Yu Y, Le X F, et al. The outcome of heregulin-induced activation of ovarian cancer cells depends on the relative levels of HER-2 and HER-3 expression. Clin Cancer Res, 1999; 5:3653-60.
Yamamoto, T., Ikawa, S., Akiyama, T., Semba, K., Nomura, N., Miyajima, N., Saito, T., and Toyoshima, K. Similarity of protein encoded by the human c-erb-B-2 gene to epidermal growth factor receptor. Nature, 319: 230-234, 1986.
Yan D H, Chang L S, and Hung M C Repressed expression of the HER-2/c-erbB-2 proto-oncogene by the adenovirus E1a gene products. Oncogene, 1991; 6:343-5.
Yan, D. H., Chang, L. S., and Hung, M. C. Repressed expression of the HER-2/c-erbB-2 proto-oncogene by the adenovirus E1a gene products. Oncogene, 6: 343-345, 1991.
Yoo G H, Hung M C, Lopez-Berestein G, et al. Phase I trial of intratumoral liposome E1A gene therapy in patients with recurrent breast and head and neck cancer. Clin Cancer Res, 2001 ; 7:1237-45.
Yoo, G. H., Hung, M. C., Lopez-Berestein, G., LaFollette, S., Ensley, J. F., Carey, M., Batson, E., Reynolds, T. C., and Murray, J. L. Phase I trial of intratumoral liposome E1A gene therapy in patients with recurrent breast and head and neck cancer. Clinical Cancer Research, 7: 1237-1245, 2001.
Young K S, Weigel R, Hiebert S, and Nevins J R Adenovirus E1A-mediated negative control of genes activated during F9 differentiation. Mol Cell Biol, 1989; 9:3109-13.
Young, K. S., Weigel, R., Hiebert, S., and Nevins, J. R. Adenovirus E1A-mediated negative control of genes activated during F9 differentiation. Mol Cell Biol, 9: 3109-3113, 1989.
Yu D, Hamada J, Zhang H, Nicolson G L, and Hung M C Mechanisms of c-erbB2/neu oncogene-induced metastasis and repression of metastatic properties by adenovirus 5 E1A gene products. Oncogene, 1992; 7:2263-70.
Yu D, Matin A, Xia W, et al. Liposome-mediated in vivo E1A gene transfer suppressed dissemination of ovarian cancer cells that overexpress HER-2/neu. Oncogene, 1995; 11: 1383-8.
Yu D, Suen T C, Yan D H, Chang L S, and Hung M C Transcriptional repression of the neu protooncogene by the adenovirus 5 E1A gene products. Proc Natl Acad Sci USA, 1990; 87:4499-503.
Yu D, Wolf J K, Scanlon M, Price J E, and Hung M C Enhanced c-erbB-2/neu expression in human ovarian cancer cells correlates with more severe malignancy that can be suppressed by E1A. Cancer Res, 1993; 53:891-8.
Yu D H, Scorsone K, and Hung M C Adenovirus type 5 E1A gene products act as transformation suppressors of the neu oncogene. Mol Cell Biol, 1991; 11:1745-50.
Yu, D. H. and Hung, M. C. Expression of activated rat neu oncogene is sufficient to induce experimental metastasis in 3T3 cells. Oncogene, 6: 1991-1996, 1991.
Yu, D. H., Liu, B. L., Tan, M., Li, J. Z., Wang, S. S., and Hung, M. C. Overexpression of c-erbb-2/neu in breast cancer cells confers increased resistance to taxol via MDR-1-independent mechanisms. Oncogene, 13: 1359-1365, 1996.
Yu, D., Hamada, J., Zhang, H., Nicolson, G. L., and Hung, M. C. Mechanisms of c-erbB2/neu oncogene-induced metastasis and repression of metastatic properties by adenovirus 5 E1A gene products. Oncogene, 7: 2263-2270, 1992.
Yu, D., Jing, T., Liu, B., Yao, J., Tan, M., McDonnell, T. J., and Hung, M. C. Overexpression of ErbB2 blocks Taxol-induced apoptosis by upregulation of p21 Cip1, which inhibits p34Cdc2 kinase. Mol Cell, 2: 581-591, 1998.
Yu, D., Liu, B., Jing, T., Sun, D., Price, J. E., Singletary, S. E., Ibrahim, N., Hortobagyi, G. N., and Hung, M. C. Overexpression of both p185c-erbB2 and p170mdr-1 renders breast cancer cells highly resistant to taxol. Oncogene, 16: 2087-2094, 1998.
Yu, D., Matin, A., Xia, W., Sorgi, F., Huang, L., and Hung, M. C. Liposome-mediated in vivo E1A gene transfer suppressed dissemination of ovarian cancer cells that overexpress HER-2/neu. Oncogene, 11: 1383-1388, 1995.
Yu, D., Suen, T. C., Yan, D. H., Chang, L. S., and Hung, M. C. Transcriptional repression of the neu protooncogene by the adenovirus 5 E1A gene products. Proc Natl Acad Sci USA, 87: 4499-4503, 1990.
Yu, D., Wang, S. S., Dulski, K. M., Tsai, C. M., Nicolson, G. L., and Hung, M. C. c-erbB-2/neu overexpression enhances metastatic potential of human lung cancer cells by induction of metastasis-associated properties. Cancer Res, 54: 3260-3266, 1994.
Zang R Y, Shi D R, Lu H J, et al. Adenovirus 5 E1a-mediated gene therapy for human ovarian cancer cells in vitro and in vivo. Int J Gynecol Cancer, 2001; 11:18-23.
Zhang Y, Yu D, Xia W, and Hung M C HER-2/neu-targeting cancer therapy via adenovirus-mediated E1A delivery in an animal model. Oncogene, 1995; 10:1947-54.
Zhang, Y., Yu, D., Xia, W., and Hung, M. C. HER-2/neu-targeting cancer therapy via adenovirus-mediated E1A delivery in an animal model. Oncogene, 10: 1947-1954, 1995.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1.-25. (canceled)

26. A method for repressing or inhibiting transformation in a breast or ovarian cell of an individual, comprising administering an effective amount of PEA15 or a mutant thereof, wherein said mutant comprises a mutation at one or more of Ser25, Ser104, or Ser116.

27. The method of claim 26, wherein said repressing or inhibiting transformation occurs in a breast cell of the individual.

28. The method of claim 26, wherein said repressing or inhibiting transformation occurs in an ovarian cell of the individual.

29. The method of claim 26, wherein the PEA15 polypeptide is administered through the introduction of a polynucleotide to the individual.

30. The method of claim 29, wherein the polynucleotide is located on a vector.

31. The method of claim 30, wherein the vector comprises a plasmid vector or a viral vector.

32. The method of claim 31, wherein the viral vector comprises a retroviral vector, and adenoviral vector, an adeno-associated viral vector, a lentiviral vector, a herpes viral vector, or a vaccinia viral vector.

33. The method of claim 26, wherein the PEA15 or mutant thereof is complexed with a liposome.

34. The method of claim 33, wherein the liposome comprises DOTMA, DOPE, DC-Chol, or LPD.

35. The method of claim 34, wherein the liposome comprises LPD.

36. The method of claim 26, wherein the repression or inhibition of the transformed phenotype is further defined as comprising sequestration of ERK in the cytoplasm of the cell.

37. The method of claim 26, wherein said method further comprises the step of subjecting the individual to an additional cancer therapy.

38. The method of claim 37, wherein the additional cancer therapy comprises surgery, chemotherapy, radiation, immunotherapy, hormonal therapy, or a combination thereof.

39. A method for facilitating or enhancing sensitivity of a breast or ovarian cancer cell in an individual to a chemotherapeutic agent, comprising the step of administering an effective amount of PEA15 or a mutant thereof to the individual, wherein said mutant comprises a mutation at one or more of Ser25, Ser104, or Ser116.

40. The method of claim 39, wherein the method is for facilitating or enhancing sensitivity of a breast cancer cell in the individual.

41. The method of claim 39, wherein the method is for facilitating or enhancing sensitivity of an ovarian cancer cell in the individual.

42. The method of claim 39, wherein the chemotherapeutic agent is paclitaxel.

43. A composition comprising an isolated PEA15 mutant polypeptide, as exemplified by SEQ ID NO:19, wherein said polypeptide comprises a mutation at one or more of Ser 25, Ser 104, or Ser116.
